(19) United States
(12) Patent Application Publication
(10) Pub. No.: US 2026/0091020 A1
(43) Pub. Date: Apr. 2, 2026

(54) TREATMENT OF ACUTE CANNABINOID OVERDOSE

(71) Applicant: Anebulo Pharmaceuticals, Inc., Lakeway, TX (US)

(72) Inventors: Joseph Fenton LAWLER, Lakeway, TX (US); Daniel Pawel SCHNEEBERGER, Austin, TX (US); Kenneth C. CUNDY, Atherton, CA (US)

(21) Appl. No.: 19/113,693

(22) PCT Filed: Sep. 25, 2023
(86) PCT No.: PCT/US2023/075053

§ 371 Date: Mar. 20, 2025

Related U.S. Application Data
(60) Provisional application No. 63/377,184, filed on Sep. 26, 2022.
(60) Provisional application No. 63/488,654, filed on Mar. 6, 2023.
(60) Provisional application No. 63/579,443, filed on Aug. 29, 2023.

(51) Int. Cl. A61K 31/397; A61K 31/00; A61P 25/30

(52) U.S. Cl. CPC A61K 31/397; A61K 31/658; A61P 25/30

(57) ABSTRACT

Provided herein are methods of treating cannabinoid intoxication. Further provided herein are metrics for evaluation Cannabis intoxication and the amelioration thereof.

### CROSS-REFERENCE TO RELATED APPLICATIONS

This application claims the benefit of U.S. Provisional Application No. 63/377,184, filed Sep. 26, 2022, U.S. Provisional Application No. 63/488,654, filed Mar. 6, 2023, and U.S. Provisional Application No. 63/579,443, filed Aug. 29, 2023, all of which are incorporated herein by reference.

### BACKGROUND

The widespread use of Δ9-tetrahydrocannabinol (THC) and synthetic cannabinoids (SCs) has resulted in an increased number of emergency room visits secondary to symptoms of cannabinoid overdose; this is especially notable after Cannabis is legalized in a jurisdiction. A medical need therefore exists to treat THC and SC related-overdoses.

### SUMMARY OF THE INVENTION

Provided herein are methods of treating a known or suspected acute cannabinoid intoxication in a subject in need thereof, the method comprising: administering to the subject a therapeutically-effective amount of a compound having the structure

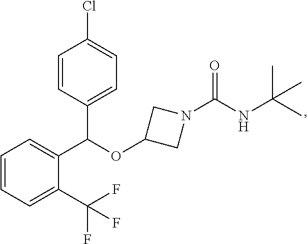 [SMILES: CC(C)(C)NC(=O)N1CC(OC(c2ccc(Cl)cc2)c2ccccc2C(F)(F)F)C1]

wherein treating improves one or more metrics associated with acute cannabinoid intoxication. Further provided herein are methods wherein the improvement is measured between a first time before administration of the compound and a second time after administration of the compound. Further provided herein are methods wherein the difference between the first time and the second time is 30 minutes to 8 hours. Further provided herein are methods wherein the time between pre-administration and post administration of the compound is 30 minutes to 4 hours. Further provided herein are methods wherein the metric comprises one or more of impaired motor coordination, and euphoria. Further provided herein are methods wherein the metric comprises heartrate, loss of alertness, body movement, or visual analog scale (VAS) assessment of cannabinoid intoxication. Further provided herein are methods wherein the loss of alertness caused by cannabinoid intoxication is reduced by at least about 20%. Further provided herein are methods wherein loss of alertness is measured by the subject's responses to one or more questions asked by a healthcare provider. Further provided herein are methods wherein the one or more questions are directed to one or more of mood and feelings. Further provided herein are methods wherein the one or more questions are directed to one or more of alertness/fuzzy/clearheaded, coordinate/clumsy, lethargic/energetic, contented/discontented, troubled/tranquil, mentally slow/quick witted, tense/relaxed, attentive/dreamy, drowsy, calm/excited, strong/feeble, incompetent/proficient, happy/sad, antagonistic/friendly, interested/bored, withdrawn/sociable, self-centered/outward going or depressed/elated. Further provided herein are methods wherein the loss of alertness caused by cannabinoid intoxication is reduced by at least one level. Further provided herein are methods wherein the loss of alertness caused by cannabinoid intoxication is reduced by at least 10%. Further provided herein are methods wherein level of alertness is scored from using best verbal response. Further provided herein are methods wherein body movement comprises body sway. Further provided herein are methods wherein body sway is measured as a distance. Further provided herein are methods wherein body sway comprises antero-posterior sway (mm). Further provided herein are methods wherein body sway distance is measured using a Wright ataxiameter. Further provided herein are methods wherein the distance of body sway distance caused by cannabinoid intoxication is reduced by at least about 25%. Further provided herein are methods wherein VAS comprises measurement of one or more of alertness, negative affect, calmness, feeling high, and external perception. Further provided herein are methods wherein VAS comprises measurement of one or more of the sixteen items in the Bond & Lader scales. Further provided herein are methods wherein the metric is measured by a healthcare provider or emergency responder. Further provided herein are methods wherein the metric is self-reported by the subject. Further provided herein are methods wherein the amount of compound administered to the subject is 1-200 mg, 10-50 mg, 10-30 mg, or 20-30 mg. Further provided herein are methods wherein the method further comprises administering 5-300 mg, 5-100 mg, or 10-30 mg of delta-9-tetrahydrocannabinol (THC) to the subject. Further provided herein are methods wherein the THC and the compound are administered at the same time. Further provided herein are methods wherein the THC is administered 10 min to 8 hours before the compound is administered. Further provided herein are methods wherein the administration is oral. Further provided herein are methods wherein the compound is administered as a capsule. Further provided herein are methods wherein the capsule comprises a soft gel capsule. Further provided herein are methods wherein the acute cannabinoid intoxication results from ingestion of an edible form of Cannabis.

Provided herein are methods of treating a known or suspected acute cannabinoid intoxication in a subject in need thereof, the method comprising: administering to the subject a therapeutically-effective amount of a compound having the structure

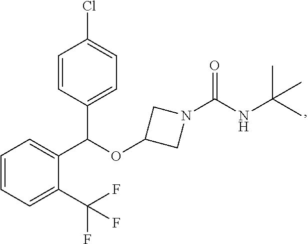 [SMILES: CC(C)(C)NC(=O)N1CC(OC(c2ccc(Cl)cc2)c2ccccc2C(F)(F)F)C1]

wherein the compound has a plasma Cₘₐₓ of about 100-500 ng/mL. Further provided herein are methods wherein the amount of compound administered to the subject is 1-200 mg, 10-50 mg, 10-30 mg, or 20-30 mg. Further provided herein are methods wherein the method further comprises administering 5-300 mg, 5-100 mg, or 10-30 mg of delta-9-tetrahydrocannabinol (THC) to the subject. Further provided herein are methods wherein the THC and the compound are administered at the same time. Further provided herein are methods wherein the THC is administered 10 min to 8 hours before the compound is administered. Further provided herein are methods wherein the administration is oral. Further provided herein are methods wherein the compound is administered as a capsule. Further provided herein are methods wherein the capsule comprises a soft gel capsule. Further provided herein are methods wherein the acute cannabinoid intoxication results from ingestion of an edible form of Cannabis.

Provided herein are methods of treating a known or suspected acute cannabinoid intoxication in a subject in need thereof, the method comprising: administering to the subject a therapeutically-effective amount of a compound having the structure

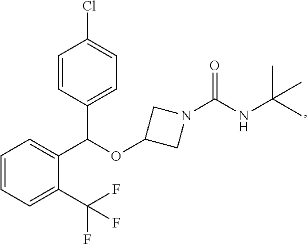 [SMILES: CC(C)(C)NC(=O)N1CC(OC(c2ccc(Cl)cc2)c2ccccc2C(F)(F)F)C1]

wherein the compound has a plasma AUCₗₐₛₜ of about 500-2800 h*ng/mL. Provided herein are methods of treating a known or suspected acute cannabinoid intoxication in a subject in need thereof, the method comprising: administering to the subject a therapeutically-effective amount of a compound having the structure

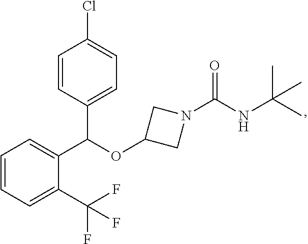 [SMILES: CC(C)(C)NC(=O)N1CC(OC(c2ccc(Cl)cc2)c2ccccc2C(F)(F)F)C1]

wherein the compound has a plasma AUC5h of about 300-1200 h*ng/mL. Further provided herein are methods wherein the amount of compound administered to the subject is 1-200 mg, 10-50 mg, 10-30 mg, or 20-30 mg. Further provided herein are methods wherein the method further comprises administering 5-300 mg, 5-100 mg, or 10-30 mg of delta-9-tetrahydrocannabinol (THC) to the subject. Further provided herein are methods wherein the THC and the compound are administered at the same time. Further provided herein are methods wherein the THC is administered 10 min to 8 hours before the compound is administered. Further provided herein are methods wherein the administration is oral. Further provided herein are methods wherein the compound is administered as a capsule. Further provided herein are methods wherein the capsule comprises a soft gel capsule. Further provided herein are methods wherein the acute cannabinoid intoxication results from ingestion of an edible form of Cannabis.

Provided herein are methods of treating a known or suspected acute cannabinoid intoxication in a subject in need thereof, the method comprising: administering to the subject a therapeutically-effective amount of a compound having the structure

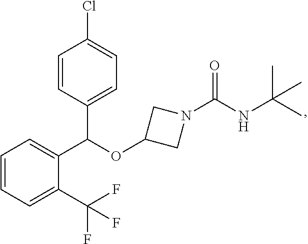 [SMILES: CC(C)(C)NC(=O)N1CC(OC(c2ccc(Cl)cc2)c2ccccc2C(F)(F)F)C1]

wherein the compound has a dose normalized plasma Cₘₐₓ of about 3-20 ng/mL/mg. Further provided herein are methods wherein the amount of compound administered to the subject is 1-200 mg, 10-50 mg, 10-30 mg, or 20-30 mg. Further provided herein are methods wherein the method further comprises administering 5-300 mg, 5-100 mg, or 10-30 mg of delta-9-tetrahydrocannabinol (THC) to the subject. Further provided herein are methods wherein the THC and the compound are administered at the same time. Further provided herein are methods wherein the THC is administered 10 min to 8 hours before the compound is administered. Further provided herein are methods wherein the administration is oral. Further provided herein are methods wherein the compound is administered as a capsule. Further provided herein are methods wherein the capsule comprises a soft gel capsule. Further provided herein are methods wherein the acute cannabinoid intoxication results from ingestion of an edible form of Cannabis.

Provided herein are methods of treating a known or suspected acute cannabinoid intoxication in a subject in need thereof, the method comprising: administering to the subject a therapeutically-effective amount of a compound having the structure

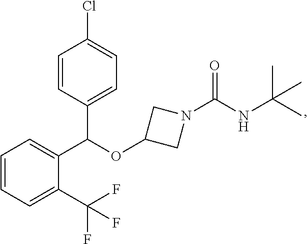 [SMILES: CC(C)(C)NC(=O)N1CC(OC(c2ccc(Cl)cc2)c2ccccc2C(F)(F)F)C1]

wherein the compound has a dose normalized plasma AUCₗₐₛₜ of about 50-400 h*ng/mL/mg. Further provided herein are methods wherein the compound has a dose normalized plasma AUCₗₐₛₜ of about 50-200 h*ng/mL/mg. Further provided herein are methods wherein the amount of compound administered to the subject is 1-200 mg, 10-50 mg, 10-30 mg, or 20-30 mg. Further provided herein are methods wherein the method further comprises administering 5-300 mg, 5-100 mg, or 10-30 mg of delta-9-tetrahydrocannabinol (THC) to the subject. Further provided herein are methods wherein the THC and the compound are administered at the same time. Further provided herein are methods wherein the THC is administered 10 min to 8 hours before the compound is administered. Further provided herein are methods wherein the administration is oral. Further provided herein are methods wherein the compound is administered as a capsule. Further provided herein are methods wherein the capsule comprises a soft gel capsule. Further provided herein are methods wherein the acute cannabinoid intoxication results from ingestion of an edible form of Cannabis.

Provided herein are methods of treating a known or suspected acute cannabinoid intoxication in a subject in need thereof, the method comprising: administering to the subject a therapeutically-effective amount of a compound having the structure

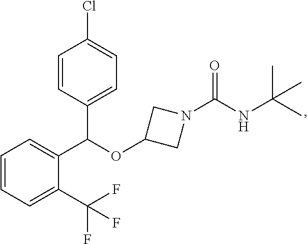 [SMILES: CC(C)(C)NC(=O)N1CC(OC(c2ccc(Cl)cc2)c2ccccc2C(F)(F)F)C1]

wherein the compound has a dosage normalized plasma AUC5h of about 10-40 h*ng/mL/mg. Further provided herein are methods wherein the amount of compound administered to the subject is 1-200 mg, 10-50 mg, 10-30 mg, or 20-30 mg. Further provided herein are methods wherein the method further comprises administering 5-300 mg, 5-100 mg, or 10-30 mg of delta-9-tetrahydrocannabinol (THC) to the subject. Further provided herein are methods wherein the THC and the compound are administered at the same time. Further provided herein are methods wherein the THC is administered 10 min to 8 hours before the compound is administered. Further provided herein are methods wherein the administration is oral. Further provided herein are methods wherein the compound is administered as a capsule. Further provided herein are methods wherein the capsule comprises a soft gel capsule. Further provided herein are methods wherein the acute cannabinoid intoxication results from ingestion of an edible form of Cannabis.

Provided herein are methods of treating a known or suspected acute cannabinoid intoxication in a subject in need thereof, the method comprising: administering to the subject a therapeutically-effective amount of a compound having the structure

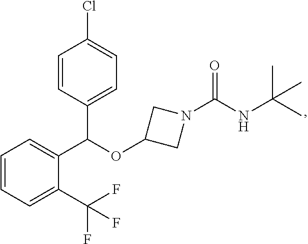 [SMILES: CC(C)(C)NC(=O)N1CC(OC(c2ccc(Cl)cc2)c2ccccc2C(F)(F)F)C1]

wherein the compound has a Tmax of or less than about 60 minutes. Further provided herein are methods wherein the amount of compound administered to the subject is 1-200 mg, 10-50 mg, 10-30 mg, or 20-30 mg. Further provided herein are methods wherein the method further comprises administering 5-300 mg, 5-100 mg, or 10-30 mg of delta-9-tetrahydrocannabinol (THC) to the subject. Further provided herein are methods wherein the THC and the compound are administered at the same time. Further provided herein are methods wherein the THC is administered 10 min to 8 hours before the compound is administered. Further provided herein are methods wherein the administration is oral. Further provided herein are methods wherein the compound is administered as a capsule. Further provided herein are methods wherein the capsule comprises a soft gel capsule. Further provided herein are methods wherein the acute cannabinoid intoxication results from ingestion of an edible form of Cannabis.

Provided herein are methods of treating a known or suspected acute cannabinoid intoxication in a subject in need thereof, the method comprising: administering to the subject a therapeutically-effective amount of a compound having the structure

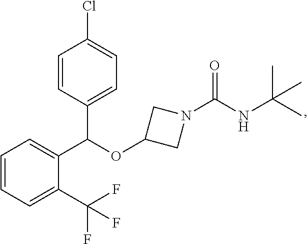 [SMILES: CC(C)(C)NC(=O)N1CC(OC(c2ccc(Cl)cc2)c2ccccc2C(F)(F)F)C1]

wherein treating accelerates recovery, shortens time in medical care or reduces the effects the intoxication as determined by an acceptable metric evaluation. Further provided herein are methods wherein the amount of compound administered to the subject is 1-200 mg, 10-50 mg, 10-30 mg, or 20-30 mg. Further provided herein are methods wherein the method further comprises administering 5-300 mg, 5-100 mg, or 10-30 mg of delta-9-tetrahydrocannabinol (THC) to the subject. Further provided herein are methods wherein the THC and the compound are administered at the same time. Further provided herein are methods wherein the THC is administered 10 min to 8 hours before the compound is administered. Further provided herein are methods wherein the administration is oral. Further provided herein are methods wherein the compound is administered as a capsule. Further provided herein are methods wherein the capsule comprises a soft gel capsule. Further provided herein are methods wherein the acute cannabinoid intoxication results from ingestion of an edible form of Cannabis.

Provided herein are metabolites of N-(tert-butyl)-3-((4-chlorophenyl)(2-(trifluoromethyl)phenyl)methoxy)azetidine-1-carboxamide. Provided herein are compositions comprising a compound having the structure:

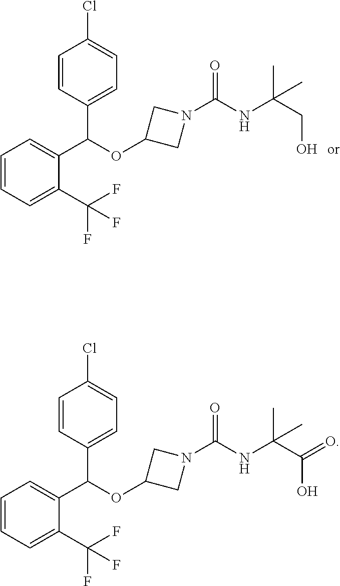 [SMILES: CC(C)(CO)NC(=O)N1CC(OC(c2ccc(Cl)cc2)c2ccccc2C(F)(F)F)C1 ; CC(C)(NC(=O)N1CC(OC(c2ccc(Cl)cc2)c2ccccc2C(F)(F)F)C1)C(=O)O]

Further provided herein are compositions wherein the compound is a salt, polymorph, solvate, hydrate, stereoisomer, or isotope thereof. Further provided herein are compositions wherein the compound is a solid. Further provided herein are compositions wherein the compound is a crystalline solid.

Provided herein are pharmaceutical compositions comprising a metabolite of a compound described herein and a pharmaceutically acceptable excipient. Provided herein are oral dosage forms of a metabolite described herein and a pharmaceutically acceptable excipient. Provided herein are injectable dosage form of a metabolite described herein and a pharmaceutically acceptable excipient. Provided herein are methods of treating a known or suspected acute cannabinoid intoxication in a subject in need thereof, the method comprising: administering to the subject a therapeutically-effective amount of a metabolite described herein.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference.

### DESCRIPTION OF THE DRAWINGS

The novel features of the present disclosure are set forth with particularity in the appended claims. An understanding of the features and advantages of the present disclosure may be obtained by reference to the following detailed description that sets forth illustrative embodiments, in which the principles of the present disclosure are utilized, and the accompanying drawings of which:

FIG. 1 depicts a randomized, double-blind, placebo controlled study. Left to right: (a) 60 Adult volunteers ages 18-45 are selected (20 per arm); (b) A single oral dose and 24-hour assessment is conducted for groups (1) 10.5 mg THC+placebo; (2) 10.5 mg THC+50 mg ANEB-001; and (3) 10.5 mg THC+100 mg ANEB-001; (c) 14 day follow up is conducted. Primary endpoints include inhibition of central nervous system effects of THC using (a) visual analog scale “feeling high”; (b) visual analog scale “alertness”; (c) body sway; (d) heart rate. Secondary endpoints include additional efficacy metrics, safety/tolerability, PK, and PK/PD correlations.

FIG. 2A depicts a plot of the time course of feeling high for groups: THC/placebo (circles); THC/50 mg ANEB-001 (squares); and THC/100 mg ANEB-001 (triangles). The y-axis is labeled VAS Feeling High log(mm) from 0.0 to 1.5 at 0.5 unit intervals, and the x-axis is labeled Time (hours) from 0 to 8 at 2 unit intervals. Data are least squares mean, 95% CI; #p<0.0001 for both dose levels; **p<0.01 for 50 mg, p<0.05 for 100 mg.

FIG. 2B depicts a bar plot of the time course feeling high (based on a score of at least 20/100 on the VAS feeling high scale) for the groups THC/placebo (left, 75%); THC/50 mg ANEB-001 (middle, 10%); and THC/100 mg ANEB-001 (right, 30%) bars. The y-axis is labeled proportion (%) from 0 to 80 at 20 unit intervals.

FIG. 2C depicts a plot of maximum VAS feeling high scores for the groups THC/placebo (left); THC/50 mg ANEB-001 (middle); and THC/100 mg ANEB-001 (right) bars. The y-axis is labeled Max VAS score (mm) from 0 to 100 at 20 unit intervals. The dashed line represents 20 mm.

FIG. 2D depicts a bar plot of proportion of subjects reporting feeling high (based on a score of at least 25/100 on the VAS feeling high scale) for the groups THC/placebo (left); THC/50 mg ANEB-001 (middle); and THC/100 mg ANEB-001 (right) bars. The y-axis is labeled proportion (%) from 0 to 80 at 20 unit intervals.

FIG. 2E depicts a plot of maximum VAS feeling high scores for the groups THC/placebo (left); THC/50 mg ANEB-001 (middle); and THC/100 mg ANEB-001 (right). The y-axis is labeled Max VAS score (mm) from 0 to 100 at 20 unit intervals. The dashed line represents 25 mm.

FIG. 2F depicts a bar plot of Responder Rate for Reduction in Feeling High for the groups THC/placebo (left); THC/50 mg ANEB-001 (middle); and THC/100 mg ANEB-001 (right) bars. The y-axis is labeled responders (%) from 0 to 80 at 20 unit intervals. *Compared to mean VAS score in the THC/placebo arm.

FIG. 3 depicts a plot of the time course of alertness for groups: THC/placebo (circles); THC/50 mg ANEB-001 (squares); and THC/100 mg ANEB-001 (triangles). The y-axis is labeled VAS alertness (mm) from 35 to 55 at 5 unit intervals, and the x-axis is labeled Time (hours) from 0 to 8 at 2 unit intervals. Data are least squares mean, 95% CI; **p<0.01 for both dose levels; *p<0.05 for both dose levels.

FIG. 4A depicts a graph of maximum change from baseline in heart rate for the groups THC/placebo (left); THC/50 mg ANEB-001 (middle); and THC/100 mg ANEB-001 (right). Administration of oral THC alone produced an increase in heart rate. The y-axis is labeled CFB hear rate (bpm) from 0 to 20 at 5 unit intervals. Coadministration of THC with ANEB-001 showed a significant reduction in the maximum change from baseline for heart rate. The 50 mg dose of ANEB-001 produced a similar effect to the 100 mg dose. Data are mean, 95% CI; *p<0.05, unpaired t-test.

FIG. 4B depicts a plot of time course of body sway (mean, 95% CI) for groups: THC/placebo (circles); THC/50 mg ANEB-001 (squares); and THC/100 mg ANEB-001 (triangles). The y-axis is labeled body sway (mm) from 200 to 400 at 50 unit intervals, and the x-axis is labeled Time (hours) from 0 to 8 at 2 unit intervals. *p<0.05 for 100 mg ANEB-001.

FIG. 5A depicts a plot of time course of heart rate for groups: THC/placebo (circles); THC/50 mg ANEB-001 (squares); and THC/100 mg ANEB-001 (triangles). The y-axis is labeled heart rate in supine position (bpm) from 55 to 70 at 5 unit intervals, and the x-axis is labeled Time (hours) from 0 to 8 at 2 unit intervals. Data are least squares mean, 95% CI, **p<0.01 for 50 mg dose, *p<0.05 for 50 mg dose.

FIG. 5B depicts a plot of time course of heart rate (mean, 95% CI) for groups: THC/placebo (circles); THC/50 mg ANEB-001 (squares); and THC/100 mg ANEB-001 (triangles). The y-axis is labeled heart rate in supine position (bpm) from 55 to 70 at 5 unit intervals, and the x-axis is labeled Time (hours) from 0 to 24 at 2 unit intervals.

FIG. 5C depicts a graph of maximum change from baseline in body sway for the groups THC/placebo (left); THC/50 mg ANEB-001 (middle); and THC/100 mg ANEB-001 (right). Administration of oral THC alone produced an increase in body sway. The y-axis is labeled max CFB body sway (mm) from 0 to 400 at 100 unit intervals. Coadministration of THC with ANEB-001 showed a significant reduction in the maximum change from baseline for body sway. The 50 mg dose of ANEB-001 produced a similar effect to the 100 mg dose. Data are mean, 95% CI; *p<0.05, unpaired t-test.

FIG. 6 depicts a plot of time course of THC-induced perception changes for groups: THC/placebo (circles); THC/50 mg ANEB-001 (squares); and THC/100 mg ANEB-001 (triangles). The y-axis is labeled VAS external perception log (mm) from 0.2 to 0.8 at 0.2 unit intervals, and the x-axis is labeled Time (hours) from 0 to 8 at 2 unit intervals. #p<0.0001; **p<0.01.

FIG. 7 depicts a plot of time course of THC-induced perception changes for groups: THC/placebo (circles); THC/50 mg ANEB-001 (squares); and THC/100 mg ANEB-001 (triangles). The y-axis is labeled VAS internal perception log (mm) from 0.25 to 0.45 at 0.05 unit intervals, and the x-axis is labeled Time (hours) from 0 to 8 at 2 unit intervals. #p<0.0001; **p<0.01.

FIG. 8 depicts a plot of time course of THC-induced mood changes for groups: THC/placebo (circles); THC/50 mg ANEB-001 (squares); and THC/100 mg ANEB-001 (triangles). The y-axis is labeled VAS mood (mm) from 50.0 to 60.0 at 2.5 unit intervals, and the x-axis is labeled Time (hours) from 0 to 8 at 2 unit intervals.

FIG. 9 depicts a plot of time course of THC-induced calmness changes for groups: THC/placebo (circles); THC/50 mg ANEB-001 (squares); and THC/100 mg ANEB-001 (triangles). The y-axis is labeled VAS calmness (mm) from 50 to 66 at 5 unit intervals, and the x-axis is labeled Time (hours) from 0 to 8 at 2 unit intervals. *p<0.05 for both dose levels.

FIG. 10 depicts items for a VAS Bond and Lader assessment. The items comprise a set of subjective VAS assessments of alertness, mood, and calmness. Composite outcomes based on subsets of 16 different VAS scales. Nine VAS items are combined to assess alertness, five VAS items are combined to assess mood, and two VAS items are combined to assess calmness. Each VAS scale consists of a 100 mm horizontal line. Two words representing opposite feelings are placed to the left and right of the horizontal line.

FIG. 11 depicts items for a VAS Bowdle assessment. No validated VAS is available for the Dutch language and population, but a translated version of the scales originally developed by Bowdle et al. has been used at CHDR to study CNS-active drug effects in multiple studies. Bowdle Psychotomimetic Effects Scores consist of thirteen 10 cm visual analogue lines ranging from 0 (‘not at all’) to 100 mm (‘extremely’), addressing various abnormal states of mind.

FIG. 12 depicts a plot of effect on nausea for groups: THC/placebo (circles); THC/50 mg ANEB-001 (squares); and THC/100 mg ANEB-001 (triangles). The y-axis is labeled VAS nausea log (mm) from 0.2 to 1 at 0.2 unit intervals, and the x-axis is labeled Time (hours) from 0 to 8 at 2 unit intervals.

FIG. 13A depicts a plot of Compound 1 (ANEB-001) plasma pharmacokinetics for a 50 mg (squares) and a 100 mg (triangles) oral dose of Compound 1 administered with 10 mg THC. The y-axis is labeled Compound 1 concentration in plasma (ng/mL) from 0 to 1000 at 250 unit intervals and the x-axis is labeled protocol time since first dose (h) from 0 to 24 at 1 hour intervals (from 0 to 5).

FIG. 13B depicts a box plot of Compound 1 (ANEB-001) plasma AUCₗₐₛₜ resulting from dosage of 50 mg (left) or 100 mg (right) of Compound 1 and 10 mg THC. The y-axis is labeled AUC_{5H} (h*ng/mL) from 5000 to 25000 at 5000 unit intervals.

FIG. 13C depicts a box plot of Compound 1 (ANEB-001) plasma Cₘₐₓ resulting from dosage of 50 mg (left) or 100 mg (right) of Compound 1 and 10 mg THC. The y-axis is labeled Cₘₐₓ (ng/mL) from 250 to 1500 at 250 unit intervals.

FIG. 13D depicts a plot of Compound 1 (ANEB-001) plasma tₘₐₓ resulting from dosage of 50 mg (left) or 100 mg (right) of Compound 1 and 10 mg THC. The y-axis is labeled tₘₐₓ (h) from 1 to 3 at 0.5 unit intervals.

FIG. 13E depicts a box plot of Compound 1 (ANEB-001) normalized plasma AUC_{inf} resulting from dosage of 50 mg (left) or 100 mg (right) of Compound 1 and 10 mg THC. The y-axis is labeled normalized AUC_{inf} (h*ng/mL/mg) from 100 to 300 at 50 unit intervals.

FIG. 13F depicts a box plot of Compound 1 (ANEB-001) plasma normalized Cₘₐₓ resulting from dosage of 50 mg (left) or 100 mg (right) of Compound 1 and 10 mg THC. The y-axis is labeled normalized Cₘₐₓ (ng/mL/mg) from 5 to 15 at 2.5 unit intervals.

FIG. 14 depicts a randomized, double-blind, placebo controlled study. Left to right. (a) 14 Adult volunteers ages 18-45 are selected; (b) A single oral dose and 24-hour assessment is conducted for groups (1) 21 mg THC+placebo; and (2) 21 mg THC+30 mg ANEB-001 (c) 7 to 14 day follow up is conducted. Primary endpoints include inhibition of central nervous system effects of THC using (a) visual analog scale “feeling high”; (b) visual analog scale “alertness”; (c) body sway; (d) heart rate. Secondary endpoints include additional efficacy metrics, safety/tolerability, PK, and PK/PD correlations.

FIG. 15 depicts a randomized, double-blind, placebo controlled study. Left to right: (a) 11 Adult volunteers ages 18-45 are selected; (b) A single oral dose and 24-hour assessment is conducted for groups (1) 21 mg THC+placebo; and (2) 21 mg THC+10 mg ANEB-001 (c) 7 to 14 day follow up is conducted. Primary endpoints include inhibition of central nervous system effects of THC using (a) visual analog scale “feeling high”; (b) visual analog scale “alertness”; (c) body sway; (d) heart rate. Secondary endpoints include additional efficacy metrics, safety/tolerability, PK, and PK/PD correlations.

FIG. 16A depicts a plot of THC-induced body sway for groups: THC/placebo (circles); and THC/30 mg ANEB-001 (squares). The y-axis is labeled body sway (mm) from 0 to 1000 at 500 unit intervals, and the x-axis is labeled Time (hours) from 0 to 8 at 2 unit intervals. Data are least squares mean, 95% CI.

FIG. 16B depicts a plot of the time course of alertness for groups: THC/placebo (circles); and THC/30 mg ANEB-001 (squares). The y-axis is labeled VAS alertness (mm) from 20 to 60 at 10 unit intervals, and the x-axis is labeled Time (hours) from 0 to 8 at 2 unit intervals. Data are least squares mean, 95% CI; **p<0.01 for both dose levels; *p<0.05 for both dose levels.

FIG. 16C depicts a plot of Responder Rate for Reduction in Feeling High for the groups THC/placebo (left); and THC/30 mg ANEB-001 (right) bars. The y-axis is labeled VAS feeling high log(mm) from 0.0 to 2.0 at 0.5 unit intervals, and the x-axis is labeled Time (hours) from 0 to 8 at 2 unit intervals.

FIG. 17A depicts a plot of Compound 1 (ANEB-001) plasma pharmacokinetics for a 30 mg oral dose of Compound 1 administered with 21 mg THC. The y-axis is labeled ANEB-001 concentration in plasma (ng/mL) from 0 to 400 at 100 unit intervals and the x-axis is labeled protocol time since first dose (h) from 0 to 24 at 4 hour intervals.

FIG. 17B depicts an expanded view of FIG. 17A. The y-axis is labeled ANEB-001 concentration in plasma (ng/mL) from 30 to 300 at 100 unit intervals and the x-axis is labeled protocol time since first dose (h) from 0 to 24 at 4 hour intervals.

FIG. 17C depicts a box plot of Compound 1 (ANEB-001) plasma AUC_{5H} resulting from dosage of 30 mg of Compound 1 and 21 mg THC. The y-axis is labeled AUC_{5H} (h*ng/mL) from 600 to 1000 at 200 unit intervals.

FIG. 17D depicts a box plot of Compound 1 (ANEB-001) plasma AUCₗₐₛₜ resulting from dosage of 30 mg of Compound 1 and 21 mg THC. The y-axis is labeled AUCₗₐₛₜ(h*ng/mL) from 1000 to 2000 at 500 unit intervals.

FIG. 17E depicts a box plot of Compound 1 (ANEB-001) plasma Cₘₐₓ resulting from dosage of 30 mg of Compound 1 and 21 mg THC. The y-axis is labeled Cₘₐₓ (ng/mL) from 200 to 500 at 100 unit intervals.

FIG. 18A depicts a plot of THC plasma pharmacokinetics for either (A) 30 mg Compound 1 (ANEB-001)+21 mg THC or (B) 21 mg THC+placebo. The y-axis is labeled THC concentration in plasma (ng/mL) from 0.0 to 10.0 at 2.5 unit intervals and the x-axis is labeled protocol time since first dose (h) from 0 to 24 at 4 hour intervals.

FIG. 18B depicts a plot of THC plasma pharmacokinetics for either (A) 30 mg Compound 1 (ANEB-001)+21 mg THC or (B) 21 mg THC+placebo. The y-axis is labeled THC concentration in plasma (ng/mL) from 0.1 to 10.0 using a base 10 log scale, and the x-axis is labeled protocol time since first dose (h) from 0 to 24 at 4 hour intervals.

FIG. 18C depicts a box plot of THC plasma AUC₅ₕ resulting from either (left) 30 mg Compound 1 (ANEB-001)+21 mg THC or (right) 21 mg THC+placebo. The y-axis is labeled AUC_{5H} (h*ng/mL) from 10 to 30 at 10 unit intervals.

FIG. 18D depicts a box plot of THC plasma AUCₗₐₛₜ resulting from either (left) 30 mg Compound 1 (ANEB-001)+21 mg THC or (right) 21 mg THC+placebo. The y-axis is labeled AUC_{5H} (h*ng/mL) from 10 to 50 at 10 unit intervals.

FIG. 18E depicts a box plot of THC plasma Cₘₐₓ resulting from either (left) 30 mg Compound 1 (ANEB-001)+21 mg THC or (right) 21 mg THC+placebo. The y-axis is labeled Cₘₐₓ (ng/mL) from 5.0 to 12.5 at 2.5 unit intervals.

FIG. 19A depicts a plot of THC plasma pharmacokinetics for either (A) 10 mg Compound 1 (ANEB-001)+21 mg THC or (B) 21 mg THC+30 mg Compound 1 (ANEB-001). The y-axis is labeled THC concentration in plasma (ng/mL) from 0 to 400 at 100 unit intervals and the x-axis is labeled protocol time since first dose (h) from 0 to 24 at 4 hour intervals.

FIG. 19B depicts a plot of THC plasma pharmacokinetics for either (A) 10 mg Compound 1 (ANEB-001)+21 mg THC or (B) 21 mg THC+30 mg Compound 1 (ANEB-001). The y-axis is labeled THC concentration in plasma (ng/mL) from 0 to 300.0 using a partial base 10 log scale, and the x-axis is labeled protocol time since first dose (h) from 0 to 24 at 4 hour intervals.

FIG. 19C depicts a box plot of THC plasma AUC₅ₕ resulting from either (left) 10 mg Compound 1 (ANEB-001)+21 mg THC or (right) 21 mg THC+30 mg Compound 1 (ANEB-001). The y-axis is labeled AUC_{5H} (h*ng/mL) from 200 to 100 at 200 unit intervals.

FIG. 19D depicts a box plot of THC plasma AUCₗₐₛₜ resulting from either (left) 10 mg Compound 1 (ANEB-001)+21 mg THC or (right) 21 mg THC+30 mg Compound 1 (ANEB-001). The y-axis is labeled AUC_{5H} (h*ng/mL) from 500 to 2000 at 500 unit intervals.

FIG. 19E depicts a box plot of THC plasma Cₘₐₓ resulting from either (left) 10 mg Compound 1 (ANEB-001)+21 mg THC or (right) 21 mg THC+30 mg Compound 1 (ANEB-001). The y-axis is labeled Cₘₐₓ (ng/mL) from 100 to 500 at 100 unit intervals.

FIG. 20 depicts a time course of Responder Rate for Reduction in Feeling High for the groups THC 21 mg/placebo (circles); THC 21 mg/10 mg ANEB-001 (squares) and THC 21 mg/30 mg ANEB-001 (triangles). The y-axis is labeled VAS feeling high log(mm) from 0.0 to 2.0 at 0.5 unit intervals, and the x-axis is labeled Time (hours) from 0 to 8 at 2 unit intervals.

FIG. 21 depicts a time course of Responder Rate for Alertness for the groups THC 21 mg/placebo (circles); THC 21 mg/10 mg ANEB-001 (squares), and THC 21 mg/30 mg ANEB-001 (triangles). The y-axis is labeled VAS alertness (mm) from 0 to 60 at 10 unit intervals, and the x-axis is labeled Time (hours) from 0 to 8 at 2 unit intervals.

FIG. 22 depicts time course of Responder Rate for Body Sway for the groups THC 21 mg/placebo (circles); THC 21 mg/10 mg ANEB-001 (squares) and THC 21 mg/30 mg ANEB-001 (triangles). The y-axis is labeled body sway (mm) from 200 to 700 at 100 unit intervals, and the x-axis is labeled Time (hours) from 0 to 8 at 2 unit intervals.

FIG. 23 depicts time course of Responder Heart Rate for the groups THC 21 mg/placebo (circles); THC 21 mg/10 mg ANEB-001 (squares) and THC 21 mg/30 mg ANEB-001 (triangles). The y-axis is labeled heart rate (bpm) from 60 to 800 at 10 unit intervals, and the x-axis is labeled Time (hours) from 0 to 8 at 2 unit intervals.

FIG. 24 depicts a plot Responder Rate for Reduction in Feeling High vs. THC dose. ANEB-001 dosages were 0 mg (placebo, circles), 50 mg (squares), 100 mg (triangles), 10 mg (square with adjacent *), 30 mg (triangle with adjacent +). The y-axis is labeled Max VAS feeling high (mm) from 0 to 80 at 20 unit intervals, and the x-axis is labeled THC dose (mg) from 0 to 25 at 5 mg intervals.

FIG. 25A depicts a summary of time course of ANEB-001 concentration in plasma of subjects challenged with THC (linear scale) for THC 21 mg/placebo (inverted triangle); THC 21 mg/10 mg ANEB-001 (circle), THC 21 mg/30 mg ANEB-001 (square), and THC 21 mg/100 mg ANEB-001 (triangle). The y-axis is labeled ANEB-001 conc. (ng/mL) from 0 to 1200 at 200 unit intervals, and the x-axis is labeled time (hours) from 0 to 24 at 6 unit intervals.

FIG. 25B depicts a summary of time course of ANEB-001 concentration in plasma of subjects challenged with THC (log scale) for THC 21 mg/placebo (inverted triangle); THC 21 mg/10 mg ANEB-001 (circle), THC 21 mg/30 mg ANEB-001 (square), and THC 21 mg/100 mg ANEB-001 (triangle). The y-axis is labeled ANEB-001 conc. (ng/mL) from 10 to 1000 on a log 10 scale, and the x-axis is labeled time (hours) from 0 to 24 at 6 unit intervals.

FIG. 26A depicts a summary of time course of THC concentration in plasma of subjects challenged with THC (linear scale) for THC 10.5 mg/placebo (circles); THC 10.5 mg/50 mg ANEB-001 (*), THC 10.5 mg/100 mg ANEB-001 (diamonds), THC 21 mg/placebo (X); THC 21 mg/10 mg ANEB-001 (squares) and THC 21 mg/30 mg ANEB-001 (+). The y-axis is labeled THC conc. (ng/mL) from 0 to 20 at 5 unit intervals, and the x-axis is labeled time (hours) from 0 to 24 at 6 unit intervals.

FIG. 26B depicts a summary of time course of THC concentration in plasma of subjects challenged with THC (log scale) for THC 10.5 mg/placebo (circles); THC 10.5 mg/50 mg ANEB-001 (*), THC 10.5 mg/100 mg ANEB-001 (diamonds), THC 21 mg/placebo (X); THC 21 mg/10 mg ANEB-001 (squares) and THC 21 mg/30 mg ANEB-001 (+). The y-axis is labeled THC conc. (ng/mL) from 0 to 10 on a log 10 scale, and the x-axis is labeled time (hours) from 0 to 24 at 6 unit intervals.

FIG. 27 depicts a plot of the time course of feeling high for two groups: 30 mg THC/placebo (circles); and 30 mg THC/10 mg ANEB-001 (squares) where the ANEB-001 is administered one hour after administration of THC. The y-axis indicates VAS Feeling High log(mm) values from 0.0 to 2.0 at 0.5 unit intervals, and the x-axis is labeled Time (hours) from −2 to 8 at 2 unit intervals.

FIG. 28 depicts a plot of the time course of feeling high for two groups: 21 mg THC/placebo (circles); and 21 mg THC/10 mg ANEB-001 (squares) where the ANEB-001 is administered one hour after administration of THC. The y-axis indicates Feeling High log(mm) values from 0.0 to 2.0 at 0.5 unit intervals, and the x-axis is labeled Time (hours) from −2 to 8 at 2 unit intervals.

### DETAILED DESCRIPTION OF THE INVENTION

Provided herein are compositions and methods of treating cannabinoid intoxication. Further provided herein are metrics and assessments for identifying cannabinoid intoxication, and measuring amelioration of cannabinoid intoxication after treatment. Further provided herein are methods of treating acute cannabinoid intoxication caused by ingestion or inhalation of an edible form of Cannabis.

### Cannabinoid Intoxication

Provided herein are metrics for measuring cannabinoid intoxication. In some instances, cannabinoid intoxication occurs from ingestion of an edible form of Cannabis, Cannabis extract, or synthetic cannabinoid. In some instances, metrics are used for determining if a patient is intoxicated with a cannabinoid. In some instances, metrics are used by healthcare providers to determine if a cannabinoid overdose treatment is provided. In some instances, metrics are used to measure the effect of treatment on a patient suffering from cannabinoid intoxication. In some instances, metrics are obtained at a first time before administration of a treatment (i.e., pre-administration) and later obtained at a second time after administration of the treatment. Changes in metrics in some instances guides treatment decisions (e.g., releasing the patient, providing a follow up dose of a CB1 inhibitor, or other action). Metrics may generally comprise physical measurements, self-reported assessments, or answers to other written or verbal questions (e.g., cognitive measurements). Any number of metrics may be used with the methods described herein, such as 1, 2, 3, 4, 5, 10, 15, 20, or more than 50 metrics. Metrics in some instances are compared to baseline or threshold levels established for a particular patient or patient population. In some instances, baseline or threshold levels take into consideration age, weight, sex, or other factor. In some instances, administration of a treatment (e.g., Compound 1 or metabolite thereof) to a patient suffering from cannabinoid intoxication results in at least 5%, 10%, 15%, 20%, 25%, 30%, 40%, 50%, 75%, 100%, 200%, or more than 200% improvement in a metric relative to a baseline value. In some instances, improvements are defined as a metric's value trending closer to a baseline value (e.g., sobriety). In some instances, administration of a treatment (e.g., Compound 1 or metabolite thereof) to a patient suffering from cannabinoid intoxication results in about 5%, 10%, 15%, 20%, 25%, 30%, 40%, 50%, 75%, 100%, 200%, or about 200% improvement in a metric relative to a baseline value. In some instances, administration of a treatment (e.g., Compound 1 or metabolite thereof) to a patient suffering from cannabinoid intoxication results in 5-200%, 5-100%, 5-80%, 5-50%, 5-25%, 10-200%, 10-100%, 10-75%, 20-200%, 20-100%, 20-75%, 50-300%, 50-200%, 50-100%, 75-300%, 100-300%, or 150-300% improvement in a metric relative to a baseline value. In some instances the improvement is realized no more than 8, 6, 5, 4, 3, 2.5, 2, 1.5, 1.2, 1.0, 0.8, or 0.5 hours after treatment. Metrics may be defined categorically (e.g., high, medium, or low), or on a continuous scale (e.g., VAS).

Physical measurements may be used to measure cannabinoid intoxication. In some instances, physical measurements are made directly on the patient. In some instances, physical measurements are taken in response to specific actions or tasks given to the patient to complete. In some instances, physical measurements comprise measurement of body movement. In some instances, body movement comprises body sway or eye movement. In some instances, eye movement comprises Saccadic movement, adaptive tracking, or smooth pursuit. In some instances, body sway comprises antero-posterior sway. In a non-limiting measurement method, body sway is measured by a pot string meter (celesco). In a non-limiting measurement method, body sway is measured by a pot string meter based on the Wright ataxiameter (Bowdle, et al.). In an exemplary protocol with a string attached to the waist, all body movements over a period of time are integrated and expressed as mm sway. Subjects are instructed to wear a pair of comfortable, low-heeled shoes on each session. Before starting a measurement, subjects are asked to stand still and comfortable, with their feet approximately 10 cm part and their hands in a relaxed position alongside the body and eyes closed. Subjects may not talk during the measurement. The total period of body-sway measurement in some instances is two minutes. Physical measurements in some instances comprise heart rate, body temperature, blood pressure, or other physical measurement. In some instances, metrics comprise one or more of eye opening response, best motor response, and best verbal response. In some instances, administration of a treatment (e.g., Compound 1 or metabolite thereof) to a patient suffering from cannabinoid intoxication results in at least 5%, 10%, 15%, 20%, 25%, 30%, 40%, 50%, 75%, 100%, 200%, or more than 200% improvement in a physical measurement relative to a baseline value. In some instances, improvements are defined as a metric's value trending closer to a baseline value (e.g., sobriety). In some instances, administration of a treatment (e.g., Compound 1 or metabolite thereof) to a patient suffering from cannabinoid intoxication results in about 5%, 10%, 15%, 20%, 25%, 30%, 40%, 50%, 75%, 100%, 200%, or about 200% improvement in a physical measurement relative to a baseline value. In some instances, administration of a treatment (e.g., Compound 1 or metabolite thereof) to a patient suffering from cannabinoid intoxication results in 5-200%, 5-100%, 5-80%, 5-50%, 5-25%, 10-200%, 10-100%, 10-75%, 20-200%, 20-100%, 20-75%, 50-300%, 50-200%, 50-100%, 75-300%, 100-300%, or 150-300% improvement in a physical measurement relative to a baseline value. In some instances the improvement is realized no more than 8, 6, 5, 4, 3, 2.5, 2, 1.5, 1.2, 1.0, 0.8, or 0.5 hours after treatment.

Cognitive metrics may also be obtained for use with the methods described herein. Cognitive metrics in some instances are obtained by self-report via verbal interrogation of a subject, orby subject's responses to a questionnaire. In some instances, questions are answered by the subject, and another party (healthcare provider, family member, or other third party) fills out the questionnaire. Questions may address any aspect of cognition experienced by the subject, including but not limited to intoxication, anxiety, alertness, mood, or internal/external perception. In some instances, questions are directed to one or more of orientation of person, place, time, and situation. In some instances, questions are directed to levels of the Glasgow coma scale. In some instances, treatment with CB1 inhibitors described herein (e.g., Compound 1 or metabolite thereof) improves a Glasgow coma metric by at least one level. In some instances, questions are directed to one or more of alertness/fuzzy/clearheaded, coordinate/clumsy, lethargic/energetic, contented/discontented, troubled/tranquil, mentally slow/quick witted, tense/relaxed, attentive/dreamy, drowsy, calm/excited, strong/feeble, incompetent/proficient, happy/sad, antagonistic/friendly, interested/bored, withdrawn/sociable, self-centered/outward going or depressed/elated. In some instances, metrics comprise measurement of working memory. In some instances, working memory is measured using an n-back test. In some instances, the n-back test comprises a zero back, one back, or two back test. In some instances, cognitive metrics are measured using a VAS (visual analog scale) measurement system. In some instances, VAS comprises choosing a position along a line wherein each end of the line comprises an opposing/opposite feeling or state (e.g., troubled vs. tranquil, calm vs. excited). In some instances, a metric comprises the distance along the line that is chosen. In some instances, the distance on the line is measured in metric units (e.g., centimeters or millimeters) or in English units (e.g., inches). In some instances, measurements comprise use of one or more questions from the sixteen questions of the Bond and Lader test. In some instances, measurements comprise use of one or more questions from the Bond and Lader test. In some instances, measurements comprise use of four or more questions from the Bond and Lader test. In some instances, measurements comprise use of six or more questions from the Bond and Lader test. In some instances, measurements comprise use of Bowdle scales. In some instances, administration of a treatment (e.g., Compound 1 or metabolite thereof) to a patient suffering from cannabinoid intoxication results in about 5%, 10%, 15%, 20%, 25%, 30%, 40%, 50%, 75%, 100%, 200%, or about 200% improvement in a cognitive measurement relative to a baseline value. In some instances, administration of a treatment (e.g., Compound 1 or metabolite thereof) to a patient suffering from cannabinoid intoxication results in 5-200%, 5-100%, 5-80%, 5-50%, 5-25%, 10-200%, 10-100%, 10-75%, 20-200%, 20-100%, 20-75%, 50-300%, 50-200%, 50-100%, 75-300%, 100-300%, or 150-300% improvement in a cognitive measurement relative to a baseline value. In some instances the improvement is realized no more than 8, 6, 5, 4, 3, 2.5, 2, 1.5, 1.2, 1.0, 0.8, or 0.5 hours after treatment.

### Compound 1

As described herein, Compound 1 (ANEB-001) refers to (R)—N-(tert-butyl)-3-((4-chlorophenyl)(2-(trifluoromethyl)phenyl)methoxy)azetidine-1-carboxamide, which has the chemical structure as shown below:

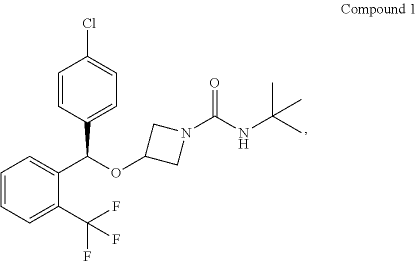 [SMILES: CC(C)(C)NC(=O)N1CC(O[C@H](c2ccc(Cl)cc2)c2ccccc2C(F)(F)F)C1]

a stereoisomer thereof or a pharmaceutically acceptable solvate or hydrate thereof.

Compound 1 is a CB1 modulator. CB1 modulators, such as a CB1 inhibitor, are useful in the treatment of various conditions and disorders, including but not limited to acute drug and Cannabis overdose and Cannabis use disorder.

The preparation and uses of Compound 1 have been previously described (see, Example 81 of U.S. Pat. No. 7,504,522, which is incorporated by reference).

In some embodiments compounds herein can include all stereoisomers, enantiomers, diastereomers, mixtures, racemates, atropisomers, and tautomers thereof.

In some embodiments as disclosed herein, Compound 1 is crystalline.

As used herein, “crystalline form,” “polymorph,” “Form,” and “form” may be used interchangeably herein, and are meant to include all crystalline and amorphous forms of the compound, including, for example, polymorphs, pseudopolymorphs, salts, solvates, hydrates, unsolvated polymorphs (including anhydrates), conformational polymorphs, and amorphous forms, as well as mixtures thereof, unless a particular crystalline or amorphous form is referred to. Compounds of the present disclosure include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. In some embodiments, the crystalline form is a single solid state form. In some instances, method described herein comprises treatment with a crystalline form of Compound 1.

Provided herein are crystalline forms of Compound 1. In some instances, a crystalline form comprises a polymorph of Compound 1. In some instances, a polymorph of Compound 1 is described in US2023/0139815, which is incorporated by reference in its entirety. In some embodiments, the crystalline form of compound 1 is Crystalline Form I. In some embodiments, Crystalline Form I is characterized by: an X-ray powder diffraction pattern comprising peaks at 10.2±0.2° 2-θ, 18.1±0.2° 2-θ, and 20.7±0.2° 2-θ, as measured by X-ray powder diffraction using an X-ray wavelength of 1.5406 Å. In some embodiments, the crystalline form is characterized by an X-ray powder diffraction pattern comprising peaks at 10.2±0.2° 2-θ, 18.1±0.2° 2-θ, and 20.7±0.2° 2-θ, and as measured by X-ray powder diffraction using an X-ray wavelength of 1.5406 Å. In some embodiments, Crystalline Form I is characterized by an X-ray powder diffraction pattern comprising peaks at about 10.2° 2-θ, about 18.1° 2-θ, and about 20.7° 2-θ, as measured by X-ray powder diffraction using an X-ray wavelength of 1.5406 Å. In some embodiments, the X-ray powder diffraction pattern further comprises at least one peak selected from 9.8±0.2° 2-θ, 15.0±0.2° 2-θ, and 22.9±0.2° 2-θ, as measured by X-ray powder diffraction using an X-ray wavelength of 1.5406 Å. In some embodiments, the X-ray powder diffraction pattern further comprises at least one peak selected from about 9.8° 2-θ, about 15.0° 2-θ, and about 22.9° 2-θ, as measured by X-ray powder diffraction using an X-ray wavelength of 1.5406 Å. In some embodiments, the X-ray powder diffraction pattern further comprises at least one peak selected from 7.1±0.2° 2-θ, 11.6±0.2° 2-θ, 13.5±0.2° 2-θ, 14.4±0.2° 2-θ, 14.6±0.2° 2-θ, 14.8±0.2° 2-θ, 16.2±0.2° 2-θ, 19.0±0.2° 2-θ, 19.3±0.2° 2-θ, 19.6±0.2° 2-θ, 20.4±0.2° 2-θ, 22.6±0.2° 2-θ, 23.2±0.2° 2-θ, and 27.7±0.2° 2-θ, as measured by X-ray powder diffraction using an X-ray wavelength of 1.5406 Å. In some embodiments, the X-ray powder diffraction pattern further comprises at least one peak selected from about 7.1° 2-θ, about 11.6° 2-θ, about 13.5° 2-θ, about 14.4° 2-θ, about 14.6° 2-θ, about 14.8° 2-θ, about 16.2° 2-θ, about 19.0° 2-θ, about 19.3° 2-θ, about 19.6° 2-θ, about 20.4° 2-θ, about 22.6° 2-θ, about 23.2° 2-θ, and about 27.7° 2-θ, as measured by X-ray powder diffraction using an X-ray wavelength of 1.5406 Å. In some embodiments, the X-ray powder diffraction pattern comprises peaks at 7.1±0.2° 2-θ, 9.8±0.2° 2-θ, 10.2±0.2° 2-θ, 11.6±0.2° 2-θ, 13.5±0.2° 2-θ, 14.4±0.2° 2-θ, 14.6±0.2° 2-θ, 14.8±0.2° 2-θ, 15.0±0.2° 2-θ, 16.2±0.2° 2-θ, 18.1±0.2° 2-θ, 19.0±0.2° 2-θ, 19.3±0.2° 2-θ, 19.6±0.2° 2-θ, 20.4±0.2° 2-θ, 20.7±0.2° 2-θ, 22.6±0.2° 2-θ, 22.9±0.2° 2-0, 23.2±0.2° 2-θ, and 27.7±0.2° 2-θ, as measured by X-ray powder diffraction using an X-ray wavelength of 1.5406 Å. In some embodiments, the X-ray powder diffraction pattern comprises at least one peak selected from 7.1±0.2° 2-θ, 9.8±0.2° 2-θ, 10.2±0.2° 2-θ, 11.6±0.2° 2-0, 13.5±0.2° 2-θ, 14.4±0.2° 2-θ, 14.6±0.2° 2-θ, 14.8±0.2° 2-θ, 15.0±0.2° 2-θ, 16.2±0.2° 2-θ, 18.1±0.2° 2-θ, 19.0±0.2° 2-θ, 19.3±0.2° 2-θ, 19.6±0.2° 2-θ, 20.4±0.2° 2-θ, 20.7±0.2° 2-θ, 22.6±0.2° 2-θ, 22.9±0.2° 2-θ, 23.2±0.2° 2-θ, and 27.7±0.2° 2-θ, as measured by X-ray powder diffraction using an X-ray wavelength of 1.5406 Å. In some embodiments, the X-ray powder diffraction pattern comprises at least two peaks selected from 7.1±0.2° 2-θ, 9.8±0.2° 2-θ, 10.2±0.2° 2-θ, 11.6±0.2° 2-θ, 13.5±0.2° 2-θ, 14.4±0.2° 2-θ, 14.6±0.2° 2-θ, 14.8±0.2° 2-θ, 15.0±0.2° 2-θ, 16.2±0.2° 2-θ, 18.1±0.2° 2-θ, 19.0±0.2° 2-θ, 19.3±0.2° 2-θ, 19.6±0.2° 2-θ, 20.4±0.2° 2-θ, 20.7±0.2° 2-θ, 22.6±0.2° 2-θ, 22.9±0.2° 2-θ, 23.2±0.2° 2-θ, and 27.7±0.2° 2-θ, as measured by X-ray powder diffraction using an X-ray wavelength of 1.5406 Å. In some embodiments, the X-ray powder diffraction pattern comprises at least three peaks selected from 7.1±0.2° 2-θ, 9.8±0.2° 2-θ, 10.2±0.2° 2-θ, 11.6±0.2° 2-θ, 13.5±0.2° 2-0, 14.4±0.2° 2-θ, 14.6±0.2° 2-θ, 14.8±0.2° 2-θ, 15.0±0.2° 2-θ, 16.2±0.2° 2-θ, 18.1±0.2° 2-θ, 19.0±0.2° 2-θ, 19.3±0.2° 2-θ, 19.6±0.2° 2-θ, 20.4±0.2° 2-θ, 20.7±0.2° 2-θ, 22.6±0.2° 2-θ, 22.9±0.2° 2-θ, 23.2±0.2° 2-θ, and 27.7±0.2° 2-θ, as measured by X-ray powder diffraction using an X-ray wavelength of 1.5406 Å. In some embodiments, the X-ray powder diffraction pattern comprises at least four peaks selected from 7.1±0.2° 2-θ, 9.8±0.2° 2-θ, 10.2±0.2° 2-θ, 11.6±0.2° 2-θ, 13.5±0.2° 2-θ, 14.4±0.2° 2-θ, 14.6±0.2° 2-θ, 14.8±0.2° 2-0, 15.0±0.2° 2-θ, 16.2±0.2° 2-θ, 18.1±0.2° 2-θ, 19.0±0.2° 2-θ, 19.3±0.2° 2-θ, 19.6±0.2° 2-θ, 20.4±0.2° 2-θ, 20.7±0.2° 2-θ, 22.6±0.2° 2-θ, 22.9±0.2° 2-θ, 23.2±0.2° 2-θ, and 27.7±0.2° 2-θ, as measured by X-ray powder diffraction using an X-ray wavelength of 1.5406 Å. In some embodiments, the X-ray powder diffraction pattern comprises at least five peaks selected from 7.1±0.2° 2-θ, 9.8±0.2° 2-θ, 10.2±0.2° 2-θ, 11.6±0.2° 2-θ, 13.5±0.2° 2-θ, 14.4±0.2° 2-θ, 14.6±0.2° 2-θ, 14.8±0.2° 2-θ, 15.0±0.2° 2-θ, 16.2±0.2° 2-θ, 18.1±0.2° 2-θ, 19.0±0.2° 2-θ, 19.3±0.2° 2-θ, 19.6±0.2° 2-θ, 20.4±0.2° 2-θ, 20.7±0.2° 2-θ, 22.6±0.2° 2-θ, 22.9±0.2° 2-θ, 23.2±0.2° 2-θ, and 27.7±0.2° 2-θ, as measured by X-ray powder diffraction using an X-ray wavelength of 1.5406 Å. In some embodiments, the X-ray powder diffraction pattern comprises at least six peaks selected from 7.1±0.2° 2-θ, 9.8±0.2° 2-θ, 10.2±0.2° 2-θ, 11.6±0.2° 2-θ, 13.5±0.2° 2-θ, 14.4±0.2° 2-θ, 14.6±0.2° 2-θ, 14.8±0.2° 2-θ, 15.0±0.2° 2-θ, 16.2±0.2° 2-θ, 18.1±0.2° 2-θ, 19.0±0.2° 2-θ, 19.3±0.2° 2-θ, 19.6±0.2° 2-θ, 20.4±0.2° 2-0, 20.7±0.2° 2-θ, 22.6±0.2° 2-θ, 22.9±0.2° 2-θ, 23.2±0.2° 2-θ, and 27.7±0.2° 2-θ, as measured by X-ray powder diffraction using an X-ray wavelength of 1.5406 Å. In some embodiments, the X-ray powder diffraction pattern comprises at least seven peaks selected from 7.1±0.2° 2-θ, 9.8±0.2° 2-θ, 10.2±0.2° 2-θ, 11.6±0.2° 2-θ, 13.5±0.2° 2-θ, 14.4±0.2° 2-0, 14.6±0.2° 2-θ, 14.8±0.2° 2-θ, 15.0±0.2° 2-θ, 16.2±0.2° 2-θ, 18.1±0.2° 2-θ, 19.0±0.2° 2-θ, 19.3±0.2° 2-θ, 19.6±0.2° 2-θ, 20.4±0.2° 2-θ, 20.7±0.2° 2-θ, 22.6±0.2° 2-θ, 22.9±0.2° 2-θ, 23.2±0.2° 2-θ, and 27.7±0.2° 2-θ, as measured by X-ray powder diffraction using an X-ray wavelength of 1.5406 Å. In some embodiments, the X-ray powder diffraction pattern comprises at least eight peaks selected from 7.1±0.2° 2-θ, 9.8±0.2° 2-θ, 10.2±0.2° 2-θ, 11.6±0.2° 2-θ, 13.5±0.2° 2-θ, 14.4±0.2° 2-θ, 14.6±0.2° 2-θ, 14.8±0.2° 2-θ, 15.0±0.2° 2-0, 16.2±0.2° 2-θ, 18.1±0.2° 2-θ, 19.0±0.2° 2-θ, 19.3±0.2° 2-θ, 19.6±0.2° 2-θ, 20.4±0.2° 2-θ, 20.7±0.2° 2-θ, 22.6±0.2° 2-θ, 22.9±0.2° 2-θ, 23.2±0.2° 2-θ, and 27.7±0.2° 2-θ, as measured by X-ray powder diffraction using an X-ray wavelength of 1.5406 Å. In some embodiments, the X-ray powder diffraction pattern comprises at least nine peaks selected from 7.1±0.2° 2-θ, 9.8±0.2° 2-θ, 10.2±0.2° 2-θ, 11.6±0.2° 2-θ, 13.5±0.2° 2-θ, 14.4±0.2° 2-0, 14.6±0.2° 2-θ, 14.8±0.2° 2-θ, 15.0±0.2° 2-θ, 16.2±0.2° 2-θ, 18.1±0.2° 2-θ, 19.0±0.2° 2-θ, 19.3±0.2° 2-θ, 19.6±0.2° 2-θ, 20.4±0.2° 2-θ, 20.7±0.2° 2-θ, 22.6±0.2° 2-θ, 22.9±0.2° 2-θ, 23.2±0.2° 2-θ, and 27.7±0.2° 2-θ, as measured by X-ray powder diffraction using an X-ray wavelength of 1.5406 Å. In some embodiments, the X-ray powder diffraction pattern comprises at least ten peaks selected from 7.1±0.2° 2-θ, 9.8±0.2° 2-θ, 10.2±0.2° 2-θ, 11.6±0.2° 2-θ, 13.5±0.2° 2-θ, 14.4±0.2° 2-θ, 14.6±0.2° 2-θ, 14.8±0.2° 2-θ, 15.0±0.2° 2-θ, 16.2±0.2° 2-θ, 18.1±0.2° 2-θ, 19.0±0.2° 2-θ, 19.3±0.2° 2-θ, 19.6±0.2° 2-θ, 20.4±0.2° 2-0, 20.7±0.2° 2-θ, 22.6±0.2° 2-θ, 22.9±0.2° 2-θ, 23.2±0.2° 2-θ, and 27.7±0.2° 2-θ, as measured by X-ray powder diffraction using an X-ray wavelength of 1.5406 Å. In some embodiments, the X-ray powder diffraction pattern comprises at least fifteen peaks selected from 7.1±0.2° 2-θ, 9.8±0.2° 2-θ, 10.2±0.2° 2-θ, 11.6±0.2° 2-θ, 13.5±0.2° 2-θ, 14.4±0.2° 2-θ, 14.6±0.2° 2-θ, 14.8±0.2° 2-θ, 15.0±0.2° 2-θ, 16.2±0.2° 2-θ, 18.1±0.2° 2-θ, 19.0±0.2° 2-θ, 19.3±0.2° 2-θ, 19.6±0.2° 2-θ, 20.4±0.2° 2-θ, 20.7±0.2° 2-θ, 22.6±0.2° 2-θ, 22.9±0.2° 2-θ, 23.2±0.2° 2-θ, and 27.7±0.2° 2-θ, as measured by X-ray powder diffraction using an X-ray wavelength of 1.5406 Å. In some embodiments, the X-ray powder diffraction pattern comprises peak at about 7.1° 2-θ, about 9.8° 2-θ, about 10.2° 2-θ, about 11.6° 2-θ, about 13.5° 2-θ, about 14.4° 2-θ, about 14.6° 2-θ, about 14.8° 2-θ, about 15.0° 2-θ, about 16.2° 2-θ, about 18.1° 2-θ, about 19.0° 2-θ, about 19.3° 2-θ, about 19.6° 2-θ, about 20.4° 2-θ, about 20.7° 2-θ, about 22.6° 2-θ, about 22.9° 2-θ, about 23.2° 2-θ, and about 27.7° 2-θ, as measured by X-ray powder diffraction using an X-ray wavelength of 1.5406 Å.

In some embodiments, the crystalline form of compound 1 is Crystalline Form II. In some embodiments, the crystalline form of compound 1 is Crystalline Form II of (R)—N-(tert-butyl)-3-((4-chlorophenyl)(2-(trifluoromethyl)phenyl)methoxy)azetidine-1-carboxamide. In some embodiments, Crystalline Form II is characterized as having an X-ray powder diffraction pattern comprising peaks at 15.2±0.2° 2-θ, 18.2±0.2° 2-θ, and 20.8±0.2° 2-0 as measured by X-ray powder diffraction using an X-ray wavelength of 1.5406 Å; In some embodiments, the crystalline form of compound 1 is Crystalline Form II of (R)—N-(tert-butyl)-3-((4-chlorophenyl)(2-(trifluoromethyl)phenyl)methoxy)azetidine-1-carboxamide. In some embodiments, Crystalline Form II is characterized by an X-ray powder diffraction pattern comprising peaks at 15.2±0.2° 2-θ, 18.2±0.2° 2-θ, and 20.8±0.2° 2-θ, as measured by X-ray powder diffraction using an X-ray wavelength of 1.5406 Å. In some embodiments, Crystalline Form II is characterized by an X-ray powder diffraction pattern comprising peaks at about 15.2° 2-θ, about 18.2° 2-θ, and about 20.8° 2-θ, as measured by X-ray powder diffraction using an X-ray wavelength of 1.5406 Å. In some embodiments, Crystalline Form II is characterized by an X-ray powder diffraction pattern further comprising at least one peak selected from 10.2±0.2° 2-θ, 19.2±0.2° 2-θ, 20.6±0.2° 2-θ, and 22.8±0.2° 2-θ, as measured by X-ray powder diffraction using an X-ray wavelength of 1.5406 Å. In some embodiments, Crystalline Form II is characterized by an X-ray powder diffraction pattern comprising peaks at about 10.2° 2-θ, about 19.2° 2-θ, about 20.6° 2-θ, and about 22.8° 2-θ, as measured by X-ray powder diffraction using an X-ray wavelength of 1.5406 Å. In some embodiments, Crystalline Form II is characterized by an X-ray powder diffraction pattern further comprising at least one peak selected from 7.0±0.2° 2-θ, 9.8±0.2° 2-θ, 13.6±0.2° 2-θ, 14.6±0.2° 2-θ, 15.0±0.2° 2-θ, 16.1±0.2° 2-θ, 19.7±0.2° 2-θ, 20.3±0.2° 2-θ, 20.4±0.2° 2-θ, as measured by X-ray powder diffraction using an X-ray wavelength of 1.5406 Å. In some embodiments, Crystalline Form II is characterized by an X-ray powder diffraction pattern fur comprising at least one peak selected from about 7.0° 2-θ, about 9.8° 2-θ, about 13.6° 2-θ, about 14.6° 2-θ, about 15.0° 2-θ, about 16.1° 2-θ, about 19.7° 2-θ, about 20.3° 2-θ, and about 20.4° 2-θ, as measured by X-ray powder diffraction using an X-ray wavelength of 1.5406 Å. In some embodiments, Crystalline Form II is characterized by an X-ray powder diffraction pattern comprising a peak selected from 7.0±0.2° 2-θ, 9.8±0.2° 2-θ, 10.2±0.2° 2-θ, 13.6±0.2° 2-θ, 14.6±0.2° 2-θ, 15.0±0.2° 2-θ, 15.2±0.2° 2-θ, 16.1±0.2° 2-θ, 18.2±0.2° 2-θ, 19.2±0.2° 2-θ, 19.7±0.2° 2-θ, 20.3±0.2° 2-θ, 20.4±0.2° 2-θ, 20.6±0.2° 2-0, 20.8±0.2° 2-θ, and 22.8±0.2° 2-θ, as measured by X-ray powder diffraction using an X-ray wavelength of 1.5406 Å. In some embodiments, Crystalline Form II is characterized by an X-ray powder diffraction pattern comprising at least one peak selected from 7.0±0.2° 2-θ, 9.8±0.2° 2-θ, 10.2±0.2° 2-θ, 13.6±0.2° 2-θ, 14.6±0.2° 2-θ, 15.0±0.2° 2-θ, 15.2±0.2° 2-θ, 16.1±0.2° 2-θ, 18.2±0.2° 2-θ, 19.2±0.2° 2-θ, 19.7±0.2° 2-θ, 20.3±0.2° 2-θ, 20.4±0.2° 2-θ, 20.6±0.2° 2-θ, 20.8±0.2° 2-θ, and 22.8±0.2° 2-θ, as measured by X-ray powder diffraction using an X-ray wavelength of 1.5406 Å. In some embodiments, Crystalline Form II is characterized by an X-ray powder diffraction pattern comprising at least two peaks selected from 7.0±0.2° 2-0, 9.8±0.2° 2-θ, 10.2±0.2° 2-θ, 13.6±0.2° 2-θ, 14.6±0.2° 2-θ, 15.0±0.2° 2-θ, 15.2±0.2° 2-0, 16.1±0.2° 2-θ, 18.2±0.2° 2-θ, 19.2±0.2° 2-θ, 19.7±0.2° 2-θ, 20.3±0.2° 2-θ, 20.4±0.2° 2-θ, 20.6±0.2° 2-θ, 20.8±0.2° 2-θ, and 22.8±0.2° 2-θ, as measured by X-ray powder diffraction using an X-ray wavelength of 1.5406 Å. In some embodiments, Crystalline Form II is characterized by an X-ray powder diffraction pattern comprising at least three peaks selected from 7.0±0.2° 2-θ, 9.8±0.2° 2-θ, 10.2±0.2° 2-θ, 13.6±0.2° 2-θ, 14.6±0.2° 2-θ, 15.0±0.2° 2-θ, 15.2±0.2° 2-θ, 16.1±0.2° 2-θ, 18.2±0.2° 2-θ, 19.2±0.2° 2-θ, 19.7±0.2° 2-θ, 20.3±0.2° 2-θ, 20.4±0.2° 2-θ, 20.6±0.2° 2-θ, 20.8±0.2° 2-θ, and 22.8±0.2° 2-θ, as measured by X-ray powder diffraction using an X-ray wavelength of 1.5406 Å. In some embodiments, Crystalline Form II is characterized by an X-ray powder diffraction pattern comprising at least four peaks selected from 7.0±0.2° 2-θ, 9.8±0.2° 2-θ, 10.2±0.2° 2-θ, 13.6±0.2° 2-θ, 14.6±0.2° 2-θ, 15.0±0.2° 2-θ, 15.2±0.2° 2-θ, 16.1±0.2° 2-θ, 18.2±0.2° 2-θ, 19.2±0.2° 2-θ, 19.7±0.2° 2-θ, 20.3±0.2° 2-θ, 20.4±0.2° 2-θ, 20.6±0.2° 2-θ, 20.8±0.2° 2-θ, and 22.8±0.2° 2-θ, as measured by X-ray powder diffraction using an X-ray wavelength of 1.5406 Å. In some embodiments, Crystalline Form II is characterized by an X-ray powder diffraction pattern comprising at least five peaks selected from 7.0±0.2° 2-θ, 9.8±0.2° 2-θ, 10.2±0.2° 2-θ, 13.6±0.2° 2-θ, 14.6±0.2° 2-θ, 15.0±0.2° 2-θ, 15.2±0.2° 2-θ, 16.1±0.2° 2-θ, 18.2±0.2° 2-0, 19.2±0.2° 2-θ, 19.7±0.2° 2-θ, 20.3±0.2° 2-θ, 20.4±0.2° 2-θ, 20.6±0.2° 2-θ, 20.8±0.2° 2-0, and 22.8±0.2° 2-θ, as measured by X-ray powder diffraction using an X-ray wavelength of 1.5406 Å. In some embodiments, Crystalline Form II is characterized by an X-ray powder diffraction pattern comprising at least six peaks selected from 7.0±0.2° 2-θ, 9.8±0.2° 2-θ, 10.2±0.2° 2-θ, 13.6±0.2° 2-θ, 14.6±0.2° 2-θ, 15.0±0.2° 2-θ, 15.2±0.2° 2-θ, 16.1±0.2° 2-0, 18.2±0.2° 2-θ, 19.2±0.2° 2-θ, 19.7±0.2° 2-θ, 20.3±0.2° 2-θ, 20.4±0.2° 2-θ, 20.6±0.2° 2-θ, 20.8±0.2° 2-θ, and 22.8±0.2° 2-θ, as measured by X-ray powder diffraction using an X-ray wavelength of 1.5406 Å. In some embodiments, Crystalline Form II is characterized by an X-ray powder diffraction pattern comprising at least seven peaks selected from 7.0±0.2° 2-θ, 9.8±0.2° 2-θ, 10.2±0.2° 2-θ, 13.6±0.2° 2-θ, 14.6±0.2° 2-θ, 15.0±0.2° 2-θ, 15.2±0.2° 2-θ, 16.1±0.2° 2-θ, 18.2±0.2° 2-θ, 19.2±0.2° 2-θ, 19.7±0.2° 2-θ, 20.3±0.2° 2-θ, 20.4±0.2° 2-0, 20.6±0.2° 2-θ, 20.8±0.2° 2-θ, and 22.8±0.2° 2-θ, as measured by X-ray powder diffraction using an X-ray wavelength of 1.5406 Å. In some embodiments, Crystalline Form II is characterized by an X-ray powder diffraction pattern comprising at least eight peaks selected from 7.0±0.2° 2-θ, 9.8±0.2° 2-θ, 10.2±0.2° 2-θ, 13.6±0.2° 2-θ, 14.6±0.2° 2-θ, 15.0±0.2° 2-θ, 15.2±0.2° 2-θ, 16.1±0.2° 2-θ, 18.2±0.2° 2-θ, 19.2±0.2° 2-θ, 19.7±0.2° 2-θ, 20.3±0.2° 2-θ, 20.4±0.2° 2-θ, 20.6±0.2° 2-θ, 20.8±0.2° 2-θ, and 22.8±0.2° 2-θ, as measured by X-ray powder diffraction using an X-ray wavelength of 1.5406 Å. In some embodiments, Crystalline Form II is characterized by an X-ray powder diffraction pattern comprising at least nine peaks selected from 7.0±0.2° 2-θ, 9.8±0.2° 2-θ, 10.2±0.2° 2-θ, 13.6±0.2° 2-θ, 14.6±0.2° 2-θ, 15.0±0.2° 2-θ, 15.2±0.2° 2-θ, 16.1±0.2° 2-θ, 18.2±0.2° 2-θ, 19.2±0.2° 2-θ, 19.7±0.2° 2-θ, 20.3±0.2° 2-θ, 20.4±0.2° 2-θ, 20.6±0.2° 2-θ, 20.8±0.2° 2-θ, and 22.8±0.2° 2-θ, as measured by X-ray powder diffraction using an X-ray wavelength of 1.5406 Å. In some embodiments, Crystalline Form II is characterized by an X-ray powder diffraction pattern comprising at least ten peaks selected from 7.0±0.2° 2-θ, 9.8±0.2° 2-θ, 10.2±0.2° 2-θ, 13.6±0.2° 2-θ, 14.6±0.2° 2-θ, 15.0±0.2° 2-θ, 15.2±0.2° 2-θ, 16.1±0.2° 2-θ, 18.2±0.2° 2-θ, 19.2±0.2° 2-θ, 19.7±0.2° 2-θ, 20.3±0.2° 2-θ, 20.4±0.2° 2-θ, 20.6±0.2° 2-θ, 20.8±0.2° 2-θ, and 22.8±0.2° 2-θ, as measured by X-ray powder diffraction using an X-ray wavelength of 1.5406 Å. In some embodiments, Crystalline Form II is characterized by an X-ray powder diffraction pattern comprising at least fifteen peaks selected from 7.0±0.2° 2-θ, 9.8±0.2° 2-θ, 10.2±0.2° 2-θ, 13.6±0.2° 2-θ, 14.6±0.2° 2-θ, 15.0±0.2° 2-θ, 15.2±0.2° 2-θ, 16.1±0.2° 2-θ, 18.2±0.2° 2-θ, 19.2±0.2° 2-θ, 19.7±0.2° 2-θ, 20.3±0.2° 2-θ, 20.4±0.2° 2-θ, 20.6±0.2° 2-θ, 20.8±0.2° 2-θ, and 22.8±0.2° 2-θ, as measured by X-ray powder diffraction using an X-ray wavelength of 1.5406 Å.

### Metabolites of Compound 1

Provided herein are metabolites of Compound 1. In some instances, a metabolite of Compound 1 comprises an oxidized form of Compound 1. In some instance, one or more functional groups of compound are oxidized to an alcohol, aldehyde, ketone, epoxide, hydroxylamine, N-oxide, imine, diol, or carboxylic acid. In some instances at least one C—H bond of Compound 1 is oxidized to an alcohol. In some instances at least two C—H bonds of Compound 1 (e.g., CH₂ group) are oxidized to a ketone. In some instances at least three C—H bonds (e.g., CH₃ group) of Compound 1 are oxidized to a carboxylic acid. In some instances at least 1, 2, 3, 4, or more functional groups are oxidized. In some instances, metabolites of Compound 1 are used in the compositions, formulations, and methods described herein.

Provided herein is a metabolite of Compound 1 is described as Compound 2 and refers to 3-((4-chlorophenyl)(2-(trifluoromethyl)phenyl)methoxy)-N-(1-hydroxy-2-methylpropan-2-yl)azetidine-1-carboxamide, which has the chemical structure as shown below:

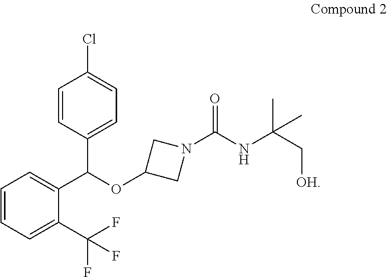 [SMILES: CC(C)(CO)NC(=O)N1CC(OC(c2ccc(Cl)cc2)c2ccccc2C(F)(F)F)C1]

Provided herein is a metabolite of Compound 1 is described as Compound 3 and refers to 2-(3-((4-chlorophenyl)(2-(trifluoromethyl)phenyl)methoxy)azetidine-1-carboxamido)-2-methylpropanoic acid, which has the chemical structure as shown below:

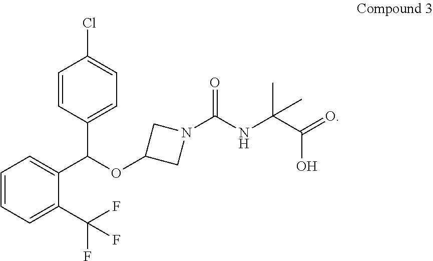 [SMILES: CC(C)(NC(=O)N1CC(OC(c2ccc(Cl)cc2)c2ccccc2C(F)(F)F)C1)C(=O)O]

In some instances, provided herein are compositions comprising a salt, polymorph, solvate, hydrate, stereoisomer, or isotope of Compounds 2 or 3. In some instances, Compounds 2 or 3 are solids. In some instances, Compounds 2 or 3 are crystalline solids. In some instances after treatment of a patient with Compound 1, at least 1%, 2%, 5%, 10%, 15%, 20%, 25%, 30%, 50%, 60%, 70%, 80%, 90%, or at least 95% of compound is converted into compounds 2 or 3 in-vivo. In some instances, the present disclosure comprises methods of synthetic preparation of compounds 2 or 3.

### Compositions and Formulations

In another aspect, the present disclosure provides compositions, including pharmaceutical composition and injectable composition.

### Pharmaceutical Composition

In one aspect, the present disclosure provides a pharmaceutical compositions comprising the (R)—N-(tert-butyl)-3-((4-chlorophenyl)(2-(trifluoromethyl)phenyl)methoxy)azetidine-1-carboxamide, and at least one pharmaceutically acceptable excipient. In some instances, a pharmaceutical composition comprises Compound 2, 3-((4-chlorophenyl)(2-(trifluoromethyl)phenyl)methoxy)-N-(1-hydroxy-2-methylpropan-2-yl)azetidine-1-carboxamide and at least one pharmaceutically acceptable excipient. In some instances, a pharmaceutical composition comprises 2-(3-((4-chlorophenyl)(2-(trifluoromethyl)phenyl)methoxy)azetidine-1-carboxamido)-2-methylpropanoic acid and at least one pharmaceutically acceptable excipient.

In some embodiments, the one or more compositions, polymorphs of Compound 1 or metabolite thereof are formulated into pharmaceutical compositions. In specific embodiments, pharmaceutical compositions are formulated in a conventional manner using one or more physiologically acceptable carriers comprising excipients and auxiliaries which facilitate processing of the active compounds/polymorphs into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In some embodiments, the present disclosure provides pharmaceutical compositions comprising Compound 1, polymorphs of Compound 1 or metabolite thereof and a pharmaceutically acceptable diluent(s), excipient(s), or carrier(s). In certain embodiments, the pharmaceutical compositions include Compound 1, or one or more polymorphs of Compound 1 or metabolite thereof.

A pharmaceutical composition, as used herein, refers to a mixture of one or more of Compound 1, polymorphs of Compound 1 or metabolite thereof with other chemical components, such as carriers, stabilizers, diluents, dispersing agents, suspending agents, thickening agents, and/or excipients. In certain embodiments, the pharmaceutical composition facilitates administration of the polymorphs to an organism. In some embodiments, in practicing the methods of treatment or use as described herein, therapeutically effective amounts of one or more of Compound 1, polymorphs of Compound 1 or metabolite thereof are administered in a pharmaceutical composition to a subject having a condition or disorder to be treated. In specific embodiments, the subject is a human. In certain embodiments, therapeutically effective amounts vary depending on the severity of the condition or disorder, the age and relative health of the subject and other factors. The one or more of Compound 1, polymorphs of Compound 1 or metabolite thereof described herein are used singly or in combination with one or more therapeutic agents as components of mixtures.

In some embodiments, Compound 1, polymorphs of Compound 1 or metabolite thereof are subjected to spray drying prior to being formulated.

In some embodiments, the pharmaceutical composition is formulated for oral, parenteral, intravenous (IV), intramuscular (IM), subcutaneous (SC), endotracheal, sublingual, buccal, intralingual, submental, transdermal, suppository, or intranasal administration.

In some embodiments, the pharmaceutical composition is formulated to deliver a therapeutically effective amount of the (R)—N-(tert-butyl)-3-((4-chlorophenyl)(2-(trifluoromethyl)phenyl)methoxy)azetidine-1-carboxamide in no more than 10 minutes. In some embodiments, the pharmaceutical composition is formulated to deliver a therapeutically effective amount of the (R)—N-(tert-butyl)-3-((4-chlorophenyl)(2-(trifluoromethyl)phenyl)methoxy)azetidine-1-carboxamide in no more than 5 minutes.

In one embodiment, the compounds and polymorphs of Compound 1 or a metabolite thereof described herein are formulated for oral administration. The compounds, polymorphs of Compound 1 or metabolite thereof are formulated by combining the compounds or polymorphs with, e.g., pharmaceutically acceptable carriers or excipients. In various embodiments, the compositions, polymorphs described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipient with one or more of the compounds or polymorphs described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of Compound 1 or polymorphs described herein is formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added. In other embodiments, Compound 1 or metabolite thereof is formulated as a softgel.

In one embodiment, Compound 1, or one or more polymorphs of Compound 1 or metabolite thereof are formulated in an aqueous solution. In other embodiments, Compound 1, or one or more polymorphs of Compound 1 or metabolite thereof are formulated for transmucosal administration. In still other embodiments wherein the one or more polymorphs described herein are formulated for other parenteral injections, appropriate formulations include aqueous or nonaqueous solutions. In specific embodiments, such solutions include physiologically compatible buffers and/or excipients.

In other embodiments, therapeutically effective amounts of at least one of the polymorphs of Compound 1 or metabolite thereof as described herein are formulated for buccal or sublingual administration. Formulations suitable for buccal or sublingual administration include, by way of example only, tablets, lozenges, or gels.

In still other embodiments, Compound 1 or the polymorphs described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical composition of a polymorph of Compound 1 or metabolite thereof is formulated in a form suitable for parenteral injection as sterile suspension, solution or emulsion in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active polymorphs in water-soluble form. In additional embodiments, suspensions of the active polymorphs are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the polymorphs to allow for the preparation of highly concentrated solutions. Alternatively, in other embodiments, the active ingredient is in powder form for constitution with a suitable vehicle, e.g., sterile pyrogen-free water, before use.

In still other embodiments, Compound 1 or polymorphs of Compound 1 or metabolite thereof are administered topically. The one or more polymorphs described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, Compound 1 or the Compound 1, or one or more polymorphs of Compound 1 or metabolite thereof are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of Compound 1 or the Compound 1, or one or more polymorphs of Compound 1 or metabolite thereof is accomplished by means of iontophoretic patches and the like. In certain embodiments, transdermal patches provide controlled delivery of Compound 1 or the Compound 1, or one or more polymorphs of Compound 1 or metabolite thereof. In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assistpassage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

In other embodiments, the Compound 1, or one or more polymorphs of Compound 1 or metabolite thereof are formulated for administration by inhalation. Various forms suitable for administration by inhalation include, but are not limited to, aerosols, mists or powders. Pharmaceutical compositions of the polymorphs of Compound 1 or metabolite thereof are conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatin for use in an inhaler or insufflator are formulated containing a powder mix of the compound and a suitable powder base such as lactose or starch.

In still other embodiments, the Compound 1, or one or more polymorphs of Compound 1 or metabolite thereof are formulated in rectal compositions for suppository administration such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active polymorphs into preparations which can be used pharmaceutically. Proper formulation is dependent upon the route of administration chosen. Any pharmaceutically acceptable techniques, carriers, and excipients are optionally used as suitable. Pharmaceutical compositions comprising the Compound 1, or one or more polymorphs of Compound 1 or metabolite thereof are manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and Compound 1 or at least one polymorph of Compound 1 or metabolite thereof described herein as an active ingredient. The active ingredient is in acid-free or base-free form, or in a pharmaceutically acceptable salt form. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions, comprising the Compound 1, or one or more polymorphs of Compound 1 or metabolite thereof described herein include formulating the polymorphs with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutically acceptable carriers or excipients for formulations described herein may comprise one or more polymers. In some embodiments, the pharmaceutically acceptable carrier is a polymer. Examples of polymers suitable for oral, buccal, intranasal, transdermal, thin-film, suppository or other administration include biocompatible and biodegradable polymers. Further examples of biocompatible polymers include natural or synthetic polymers such as polystyrene, polylactic acid, polyketal, butadiene styrene, styreneacrylic-vinyl terpolymer, polymethylmethacrylate, polyethylmethacrylate, polyalkylcyanoacrylate, styrene-maleic anhydride copolymer, polyvinyl acetate, polyvinylpyridine, polydivinylbenzene, polybutyleneterephthalate, acrylonitrile, vinylchloride-acrylates, polycaprolactone, poly(alkyl cyanoacrylates), poly(lactic-co-glycolic acid), and the like. In some instances, the carrier is Labrasol. In some instances, the carrier is methyl cellulose. In further embodiments, the pharmaceutically acceptable carrier comprises one or more biodegradable polymers. Use of biodegradable polymers provides the advantages of using a formulation that will eventually disintegrate, which facilitates release of the benzofuran compound and elimination of the carrier in vivo. However, benzofuran compounds can also be released from the matrix of non-biodegradable polymers as a result of gradual efflux from channels within the polymer matrix, including those formed by soluble materials included in the polymer matrix.

Examples of biodegradable polymers include polylactide polymers include poly(D,L-lactide)s; poly(lactide-co-glycolide) (PLGA) copolymers; polyglycolide (PGA) and polydioxanone; caprolactone polymers; chitosan; hydroxybutyric acids; polyanhydrides and polyesters; polyphosphazenes; and polyphosphoesters. In some instances, the biodegradable polymer for use in the nanoparticles is poly-(D,L-lactide-co-glycolide).

Functionalized poly(D,L-lactide)s can also be used as biodegradable polymers in the nanoparticles described herein. Examples of functionalized poly(D,L-lactide)s include poly(L-lactide), acrylate terminated; poly(L-lactide), amine terminated; poly(L-lactide), azide terminated; poly(L-lactide), 2-bromoisobutyryl terminated; poly(L-lactide), 2-bromoisobutyryl terminated; poly(L-lactide) 4-cyano-4-[(dodecylsulfanylthiocarbonyl)sulfanyl]pentonate; poly(L-lactide) N-2-hydroxyethylmaleimide terminated; poly(L-lactide) 2-hydroxyethyl, methacrylate terminated; poly(L-lactide), propargyl terminated; or poly(L-lactide), thiol terminated.

Other biodegradable polymers that can be used in the nanoparticles include AB eblock copolymers such as poly(ethylene glycol) methyl ether-block-poly(D,L-lactide); poly(ethylene glycol) methyl ether-block-poly(lactide-co-glycolide) PEG; poly(ethylene glycol)-block-poly(.epsilon.-caprolactone) methyl ether PEG; and polypyrrole-block-poly(caprolactone). Further biodegradable polymers include ABA triblock copolymers such as polylactide-block-poly(ethylene glycol)-block-polylactide PLA; poly(lactide-co-glycolide)-block-poly(ethylene glycol)-block-poly(lactide-co-glycolide); poly(lactide-co-caprolactone)-block-poly(ethylene glycol)-block-poly(lactide-co-caprolactone); poly caprolactone-block-polytetrahydrofuran-block-polycaprolactone; and polyglycolide-block-poly(ethylene glycol)-block-polyglycolide PEG.

Biodegradable polymers also include various natural polymers. Examples of natural polymers include polypeptides including those modified non-peptide components, such as saccharide chains and lipids; nucleotides; sugar-based biopolymers such as polysaccharides; cellulose; carbohydrates and starches; dextrans; lignins; polyamino acids; adhesion proteins; lipids and phospholipids (e.g., phosphorylcholine). In some embodiments, the polymer is a cellulose derivative such as hydroxypropyl methylcellulose polymers. Hydroxypropyl methyl cellulose (HPMC) is a non-ionic cellulose ether made through a series of chemical processes, with the natural polymer cellulose as the raw material. The product is a non-ionic cellulose ether in the shape of white powder, odorless and tasteless. HPMC is also known as hypromellose, is a methylcellulose modified with a small amount of propylene glycol ether groups attached to the anhydroglucose of the cellulose.

Useful pharmaceutical compositions also, optionally, include solubilizing agents to aid in the solubility of a polymorph of Compound 1 or metabolite thereof. The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

### Injectable Compositions

In some embodiments, Compound 1 or metabolite thereof is formulated into an injectable composition. In some embodiments, the injectable composition comprises (R)—N-(tert-butyl)-3-((4-chlorophenyl)(2-(trifluoromethyl)phenyl)methoxy)azetidine-1-carboxamide, or the pharmaceutical composition described herein, an opioid antagonist, and a benzodiazepine antagonist. In some embodiments, the benzodiazepine antagonist is flumazenil. In some embodiments, the opioid antagonist is naloxone or naltrexone. In some embodiments, the opioid antagonist is samidorphan. In some embodiments, the opioid antagonist is naltrexone. In some embodiments, the injectable composition is formulated in a single dose injectable device.

### Methods of Making Compound 1 and Polymorphic Forms Thereof

In another aspect, the present disclosure provides methods of making one or more polymorphs or metabolites of Compound 1:

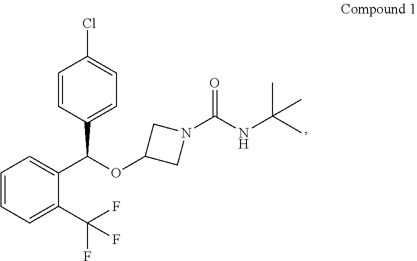 [SMILES: CC(C)(C)NC(=O)N1CC(O[C@H](c2ccc(Cl)cc2)c2ccccc2C(F)(F)F)C1]

or a pharmaceutically acceptable solvate or hydrate thereof.

The preparation and uses of Compound 1 have been previously described (see, Example 81 of U.S. Pat. No. 7,504,522, which is incorporated by reference).

### Methods of Treatment

Methods provided herein may comprise measurement of one or more metrics followed by treatment with compounds described herein (e.g., Compound 1 or a metabolite thereof). In one aspect, the present disclosure provides a method of treating known or suspected acute drug overdose reaction in a subject in need thereof, comprising administering to the subject a therapeutically effective amount of (R)—N-(tert-butyl)-3-((4-chlorophenyl)(2-(trifluoromethyl)phenyl)methoxy)azetidine-1-carboxamide, or the pharmaceutical composition as described herein. Provided herein are methods of treating a known or suspected acute cannabinoid intoxication in a subject in need thereof, the method comprising: administering to the subject a therapeutically-effective amount of a compound having the structure

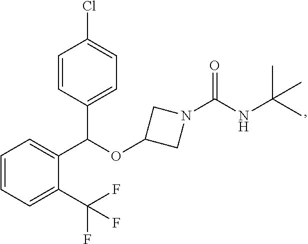 [SMILES: CC(C)(C)NC(=O)N1CC(OC(c2ccc(Cl)cc2)c2ccccc2C(F)(F)F)C1]

Provided herein are methods of treating a known or suspected acute cannabinoid intoxication in a subject in need thereof, the method comprising: administering to the subject a therapeutically-effective amount of 3-((4-chlorophenyl)(2-(trifluoromethyl)phenyl)methoxy)-N-(1-hydroxy-2-methylpropan-2-yl)azetidine-1-carboxamide and at least one pharmaceutically acceptable excipient. Provided herein are methods of treating a known or suspected acute cannabinoid intoxication in a subject in need thereof, the method comprising: administering to the subject a therapeutically-effective amount of 2-(3-((4-chlorophenyl)(2-(trifluoromethyl)phenyl)methoxy)azetidine-1-carboxamido)-2-methylpropanoic acid. In some instances, methods of treatment comprise administration of synthetically prepared compound 2 or compound 3.

In some embodiments, the subject shows signs of an acute cannabinoid overdose. In some embodiments, the acute cannabinoid overdose is caused by a compound from the Cannabis genus. In some embodiments, the acute cannabinoid overdose is caused by a synthetic cannabinoid. In some embodiments, the acute cannabinoid overdose is caused by oral ingestion of cannabinoids or synthetic cannabinoids. In some embodiments, the acute cannabinoid overdose is caused by oral ingestion of cannabinoids or synthetic cannabinoids. In some embodiments, the synthetic cannabinoid is capable of binding to the Cannabinoid (CB1) receptor. In some embodiments, the subject shows signs of cannabinoid hyperemesis syndrome.

Administration with a compound described herein (e.g., Compound 1 or metabolite thereof) to treat cannabinoid intoxication may occur immediately after exposure to Cannabis, extract thereof, or synthetic cannabinoid (e.g., first time;), or at a later time (e.g., second time). In some instances, administration (e.g., ingestion of edibles, inhalation, etc.) of Cannabis or other cannabinoid occurs at the same time as administration of a cannabinoid intoxication treatment (e.g., Compound 1 or metabolite thereof). In some instances, administration (e.g., ingestion of edibles, inhalation, etc.) of Cannabis or other cannabinoid occurs prior to treatment. In some instances, administration (e.g., ingestion of edibles, inhalation, etc.) of Cannabis or other cannabinoid occurs 0.1, 0.2, 0.5, 0.7. 1, 1.5, 2, 3, 4, 5, 6, 8, 10, 12, 14, 16, or about 20 hours prior to treatment (e.g., administration of Compound 1 or metabolite thereof). In some instances, administration (e.g., ingestion of edibles, inhalation, etc.) of Cannabis or other cannabinoid occurs at least 0.1, 0.2, 0.5, 0.7. 1, 1.5, 2, 3, 4, 5, 6, 8, 10, 12, 14, 16, or no more than 20 hours prior to treatment (e.g., administration of Compound 1 or metabolite thereof). In some instances, administration (e.g., ingestion of edibles, inhalation, etc.) of Cannabis or other cannabinoid occurs at least 0.1, 0.2, 0.5, 0.7. 1, 1.5, 2, 3, 4, 5, 6, 8, 10, 12, 14, 16, or at least 20 hours prior to treatment (e.g., administration of Compound 1 or metabolite thereof). In some instances, administration (e.g., ingestion of edibles, inhalation, etc.) of Cannabis or other cannabinoid occurs at least 0.1-20. 0.1-16, 0.1-12, 0.1-10, 0.1-8, 0.1-6, 0.1-6, 0.1-4, 0.1-1, 0.5-12, 0.5-8, 0.5-6, 0.5-4, 1-8, 1-6, 1-4. 1-2, 2-4, 2-6, 2-8, 4-12, 4-6, 4-8, 4-12, 6-12, 6-14, or 6-20 hours prior to treatment (e.g., administration of Compound 1 or metabolite thereof).

In some embodiments, the method further comprising treatment for drug overdose prior to treatment with (R)—N-(tert-butyl)-3-((4-chlorophenyl)(2-(trifluoromethyl)phenyl)methoxy)azetidine-1-carboxamide. In some embodiments, the prior treatment comprises one or more of administration of an opiate antagonist, activated charcoal, or emetic.

In another aspect, the present disclosure provides a method of using (R)—N-(tert-butyl)-3-((4-chlorophenyl)(2-(trifluoromethyl)phenyl)methoxy)azetidine-1-carboxamide, or the pharmaceutical composition as described herein, comprising administering a therapeutically effective amount of (R)—N-(tert-butyl)-3-((4-chlorophenyl)(2-(trifluoromethyl)phenyl)methoxy)azetidine-1-carboxamide prior to exposure to a cannabinoid. In some embodiments, the cannabinoid is tetrahydrocannabinol (THC).

In another aspect, the present disclosure provides a method of treating a subject suspected of a drug overdose, comprising administering a therapeutically effective amount of (R)—N-(tert-butyl)-3-((4-chlorophenyl)(2-(trifluoromethyl)phenyl)methoxy)azetidine-1-carboxamide to the subject and monitoring said subject for reduced symptoms associated with overdose. In some embodiments, the monitoring comprises monitoring heart rate or respiration.

In another aspect, the present disclosure provides a method of treating Cannabis use disorder (CUD) in a subject in need thereof, comprising administering a therapeutically effective amount of (R)—N-(tert-butyl)-3-((4-chlorophenyl)(2-(trifluoromethyl)phenyl)methoxy)azetidine-1-carboxamide, or the pharmaceutical composition as described herein. In some embodiments, the subject is addicted to a compound from the Cannabis genus. In some embodiments, the subject is addicted to a synthetic cannabinoid. In some embodiments, the synthetic cannabinoid is capable of binding to the CB1 receptor.

In various embodiments of the methods as described herein, (R)—N-(tert-butyl)-3-((4-chlorophenyl)(2-(trifluoromethyl)phenyl)methoxy)azetidine-1-carboxamide is Form I. In another embodiment, (R)—N-(tert-butyl)-3-((4-chlorophenyl)(2-(trifluoromethyl)phenyl)methoxy)azetidine-1-carboxamide is Form II.

In various embodiments of the methods described herein, the method further comprising a diagnostic test prior to treatment with (R)—N-(tert-butyl)-3-((4-chlorophenyl)(2-(trifluoromethyl)phenyl)methoxy)azetidine-1-carboxamide. In some embodiments, the diagnostic test is a blood test. In some embodiments, the subject has a cannabinoid plasma concentration of at least 25 g/L. In some embodiments, the diagnostic test is a blood test. In some embodiments, the subject has a cannabinoid plasma concentration of at least 50 g/L. In some embodiments, the subject has a cannabinoid plasma concentration of about 25 g/L to 350 g/L. In some embodiments, the subject has a cannabinoid plasma concentration of about 50 g/L to 350 g/L. In some embodiments, the subject has a cannabinoid plasma concentration of about 75 g/L to 350 g/L. In some embodiments, the subject has a cannabinoid plasma concentration of about 100 g/L to 350 g/L. In some embodiments, the subject has a cannabinoid plasma concentration of about 150 g/L to 350 g/L. In some embodiments, the subject has a cannabinoid plasma concentration of about 200 g/L to 350 g/L.

In various embodiments of the methods as described here, the amount of (R)—N-(tert-butyl)-3-((4-chlorophenyl)(2-(trifluoromethyl)phenyl)methoxy)azetidine-1-carboxamide is between about 0.1 mg to about 200 mg. In another embodiment, the amount of (R)—N-(tert-butyl)-3-((4-chlorophenyl)(2-(trifluoromethyl)phenyl)methoxy)azetidine-1-carboxamide is between about 1 mg to about 200 mg. In another embodiment, the amount of (R)—N-(tert-butyl)-3-((4-chlorophenyl)(2-(trifluoromethyl)phenyl)methoxy)azetidine-1-carboxamide is between about 0.5 mg to about 200 mg. In another embodiment, the amount of (R)—N-(tert-butyl)-3-((4-chlorophenyl)(2-(trifluoromethyl)phenyl)methoxy)azetidine-1-carboxamide is between about 2 mg to about 200 mg. In another embodiment, the amount of (R)—N-(tert-butyl)-3-((4-chlorophenyl)(2-(trifluoromethyl)phenyl)methoxy)azetidine-1-carboxamide is between about 5 mg to about 200 mg. In another embodiment, the amount of (R)—N-(tert-butyl)-3-((4-chlorophenyl)(2-(trifluoromethyl)phenyl)methoxy)azetidine-1-carboxamide is between about 10 mg to about 200 mg. In another embodiment, the amount of (R)—N-(tert-butyl)-3-((4-chlorophenyl)(2-(trifluoromethyl)phenyl)methoxy)azetidine-1-carboxamide is between about 15 mg to about 200 mg. In another embodiment, the amount of (R)—N-(tert-butyl)-3-((4-chlorophenyl)(2-(trifluoromethyl)phenyl)methoxy)azetidine-1-carboxamide is between about 20 mg to about 200 mg. In another embodiment, the amount of (R)—N-(tert-butyl)-3-((4-chlorophenyl)(2-(trifluoromethyl)phenyl)methoxy)azetidine-1-carboxamide is between about 25 mg to about 200 mg. In another embodiment, the amount of (R)—N-(tert-butyl)-3-((4-chlorophenyl)(2-(trifluoromethyl)phenyl)methoxy)azetidine-1-carboxamide is between about 30 mg to about 200 mg. In another embodiment, the amount of (R)—N-(tert-butyl)-3-((4-chlorophenyl)(2-(trifluoromethyl)phenyl)methoxy)azetidine-1-carboxamide is between about 35 mg to about 200 mg. In another embodiment, the amount of (R)—N-(tert-butyl)-3-((4-chlorophenyl)(2-(trifluoromethyl)phenyl)methoxy)azetidine-1-carboxamide is between about 40 mg to about 200 mg. In another embodiment, the amount of (R)—N-(tert-butyl)-3-((4-chlorophenyl)(2-(trifluoromethyl)phenyl)methoxy)azetidine-1-carboxamide is between about 45 mg to about 200 mg. In another embodiment, the amount of (R)—N-(tert-butyl)-3-((4-chlorophenyl)(2-(trifluoromethyl)phenyl)methoxy)azetidine-1-carboxamide is between about 50 mg to about 200 mg. In another embodiment, the amount of (R)—N-(tert-butyl)-3-((4-chlorophenyl)(2-(trifluoromethyl)phenyl)methoxy)azetidine-1-carboxamide is between about 75 mg to about 200 mg. In another embodiment, the amount of (R)—N-(tert-butyl)-3-((4-chlorophenyl)(2-(trifluoromethyl)phenyl)methoxy)azetidine-1-carboxamide is between about 100 mg to about 200 mg. In another embodiment, the amount of (R)—N-(tert-butyl)-3-((4-chlorophenyl)(2-(trifluoromethyl)phenyl)methoxy)azetidine-1-carboxamide is between about 125 mg to about 200 mg. In another embodiment, the amount of (R)—N-(tert-butyl)-3-((4-chlorophenyl)(2-(trifluoromethyl)phenyl)methoxy)azetidine-1-carboxamide is between about 150 mg to about 200 mg. In another embodiment, the amount of (R)—N-(tert-butyl)-3-((4-chlorophenyl)(2-(trifluoromethyl)phenyl)methoxy)azetidine-1-carboxamide is between about 175 mg to about 200 mg. In another embodiment, the amount of (R)—N-(tert-butyl)-3-((4-chlorophenyl)(2-(trifluoromethyl)phenyl)methoxy)azetidine-1-carboxamide is 25-500, 25-400, 25-300, 25-250, 25-200, 25-150, 25-100, or 25-75 mg. In another embodiment, the amount of (R)—N-(tert-butyl)-3-((4-chlorophenyl)(2-(trifluoromethyl)phenyl)methoxy)azetidine-1-carboxamide is 50-500, 50-400, 50-300, 50-250, 50-200, 50-150, 50-100, or 50-75 mg. In another embodiment, the amount of (R)—N-(tert-butyl)-3-((4-chlorophenyl)(2-(trifluoromethyl)phenyl)methoxy)azetidine-1-carboxamide is 75-500, 75-400, 75-300, 75-250, 75-200, 75-150, 75-100, or 75-125 mg. In another embodiment, the amount of (R)—N-(tert-butyl)-3-((4-chlorophenyl)(2-(trifluoromethyl)phenyl)methoxy)azetidine-1-carboxamide is 100-500, 100-400, 100-300, 100-250, 100-200, 100-150, 100-125 mg. In another embodiment, the amount of (R)—N-(tert-butyl)-3-((4-chlorophenyl)(2-(trifluoromethyl)phenyl)methoxy)azetidine-1-carboxamide is 1-200 mg, 10-50 mg, 10-30 mg, or 20-30 mg. In another embodiment, the amount of (R)—N-(tert-butyl)-3-((4-chlorophenyl)(2-(trifluoromethyl)phenyl)methoxy)azetidine-1-carboxamide is 5-300 mg, 5-100 mg, or 10-30 mg. In another embodiment, the amount of (R)—N-(tert-butyl)-3-((4-chlorophenyl)(2-(trifluoromethyl)phenyl)methoxy)azetidine-1-carboxamide is 1-50, 1-30, 1-25, 1-20, 1-15, 1-10, 1-5, or 1-3 mg. In another embodiment, the amount of (R)—N-(tert-butyl)-3-((4-chlorophenyl)(2-(trifluoromethyl)phenyl)methoxy)azetidine-1-carboxamide is 50, 30, 25, 20, 15, 10, 5, 3 or 1 mg.

In various embodiments, the method is capable of ameliorating one or more symptoms of the acute drug overdose reaction in no more than 30 minutes. In another embodiment, the method is capable of ameliorating one or more symptoms of the acute drug overdose reaction in no more than 1 hour. In various embodiments, the method is capable of ameliorating one or more symptoms of the acute drug overdose reaction in no more than 10 minutes. In another embodiment, the method is capable of ameliorating one or more symptoms of the acute drug overdose reaction in no more than 15 minutes. In various embodiments, the method is capable of ameliorating one or more symptoms of the acute drug overdose reaction in no more than 45 minutes. In another embodiment, the method is capable of ameliorating one or more symptoms of the acute drug overdose reaction in no more than 20 minutes. In various embodiments, the method is capable of ameliorating one or more symptoms of the acute drug overdose reaction in 5-60, 5-45, 5-30, 5-25, 5-20, 5-15, 5-10, 10-60, 10-120, 10-30, 20-60, 20-120, 50-120, 60-120, or 90-120 minutes.

Compounds described herein (e.g., Compound 1 or metabolite thereof) in some instances have desirable PK profiles for treating cannabinoid overdose. In some instances concentrations (e.g., Cmax, AUC, etc.) of Compound 1 or metabolite thereof are measured in plasma.

In various embodiments of the methods described herein, the amount of (R)—N-(tert-butyl)-3-((4-chlorophenyl)(2-(trifluoromethyl)phenyl)methoxy)azetidine-1-carboxamide in the bloodstream of the subject reaches at least about 50 ng/mL, at least about 100 ng/mL or at least about 200 ng/mL within one hour after oral administration. In another embodiment, the amount of (R)—N-(tert-butyl)-3-((4-chlorophenyl)(2-(trifluoromethyl)phenyl)methoxy)azetidine-1-carboxamide in the bloodstream of the subject reaches at least 200 ng/mL within 50 minutes after oral administration. In another embodiment, the amount of (R)—N-(tert-butyl)-3-((4-chlorophenyl)(2-(trifluoromethyl)phenyl)methoxy)azetidine-1-carboxamide in the bloodstream of the subject reaches at least 200 ng/mL within 40 minutes after oral administration. In another embodiment, the amount of (R)—N-(tert-butyl)-3-((4-chlorophenyl)(2-(trifluoromethyl)phenyl)methoxy)azetidine-1-carboxamide in the bloodstream of the subject reaches at least 200 ng/mL within 30 minutes after oral administration. In another embodiment, the amount of (R)—N-(tert-butyl)-3-((4-chlorophenyl)(2-(trifluoromethyl)phenyl)methoxy)azetidine-1-carboxamide in the bloodstream of the subject reaches at least 200 ng/mL within 20 minutes after oral administration. In another embodiment, the amount of (R)—N-(tert-butyl)-3-((4-chlorophenyl)(2-(trifluoromethyl)phenyl)methoxy)azetidine-1-carboxamide in the bloodstream of the subject reaches at least 200 ng/mL within 10 minutes after oral administration. In another embodiment, the amount of (R)—N-(tert-butyl)-3-((4-chlorophenyl)(2-(trifluoromethyl)phenyl)methoxy)azetidine-1-carboxamide in the bloodstream of the subject reaches at least 200 ng/mL within 5 minutes after oral administration.

In various embodiments of the methods described herein, the amount of (R)—N-(tert-butyl)-3-((4-chlorophenyl)(2-(trifluoromethyl)phenyl)methoxy)azetidine-1-carboxamide in the bloodstream of the subject reaches at least 200 ng/mL within one hour after oral administration. In another embodiment, the amount of (R)—N-(tert-butyl)-3-((4-chlorophenyl)(2-(trifluoromethyl)phenyl)methoxy)azetidine-1-carboxamide in the bloodstream of the subject reaches at least 150 ng/mL within 50 minutes after oral administration. In another embodiment, the amount of (R)—N-(tert-butyl)-3-((4-chlorophenyl)(2-(trifluoromethyl)phenyl)methoxy)azetidine-1-carboxamide in the bloodstream of the subject reaches at least 150 ng/mL within 40 minutes after oral administration. In another embodiment, the amount of (R)—N-(tert-butyl)-3-((4-chlorophenyl)(2-(trifluoromethyl)phenyl)methoxy)azetidine-1-carboxamide in the bloodstream of the subject reaches at least 150 ng/mL within 30 minutes after oral administration. In another embodiment, the amount of (R)—N-(tert-butyl)-3-((4-chlorophenyl)(2-(trifluoromethyl)phenyl)methoxy)azetidine-1-carboxamide in the bloodstream of the subject reaches at least 150 ng/mL within 20 minutes after oral administration. In another embodiment, the amount of (R)—N-(tert-butyl)-3-((4-chlorophenyl)(2-(trifluoromethyl)phenyl)methoxy)azetidine-1-carboxamide in the bloodstream of the subject reaches at least 150 ng/mL within 10 minutes after oral administration. In another embodiment, the amount of (R)—N-(tert-butyl)-3-((4-chlorophenyl)(2-(trifluoromethyl)phenyl)methoxy)azetidine-1-carboxamide in the bloodstream of the subject reaches at least 150 ng/mL within 5 minutes after oral administration.

In some instances, (R)—N-(tert-butyl)-3-((4-chlorophenyl)(2-(trifluoromethyl)phenyl)methoxy)azetidine-1-carboxamide is dosed to provide a Tmax of no more than 5, 4, 3, 2.5, 2.25, 2, 1.75, 1.5, 1.25, 1, 0.75, 0.5, or 0.25 hours. In some instances, (R)—N-(tert-butyl)-3-((4-chlorophenyl)(2-(trifluoromethyl)phenyl)methoxy)azetidine-1-carboxamide is dosed to provide a Tmax of0.1-5, 0.1-4, 0.1-3, 0.1-2, 0.1-1.5, 0.1-1, 0.25-3, 0.25-2, 0.25-1.5, 0.25-1, 0.5-3, 0.5-2.5, 0.5-2, 0.5-1.5, 0.5-1.25, 0.5-1, 0.5-0.75, or 0.1-0.75 hr. In some instances, (R)—N-(tert-butyl)-3-((4-chlorophenyl)(2-(trifluoromethyl)phenyl)methoxy)azetidine-1-carboxamide dosed at 100-200 mg provides a Tmax of 0.1-5, 0.1-4, 0.1-3, 0.1-2, 0.1-1.5, 0.1-1, 0.25-3, 0.25-2, 0.25-1.5, 0.25-1, 0.5-3, 0.5-2.5, 0.5-2, 0.5-1.5, 0.5-1.25, 0.5-1, 0.5-0.75, or 0.1-0.75 hr. In some instances, (R)—N-(tert-butyl)-3-((4-chlorophenyl)(2-(trifluoromethyl)phenyl)methoxy)azetidine-1-carboxamide dosed at 25-250 mg provides a Tmax of 0.1-5, 0.1-4, 0.1-3, 0.1-2, 0.1-1.5, 0.1-1, 0.25-3, 0.25-2, 0.25-1.5, 0.25-1, 0.5-3, 0.5-2.5, 0.5-2, 0.5-1.5, 0.5-1.25, 0.5-1, 0.5-0.75, or 0.1-0.75 hr. In some instances, (R)—N-(tert-butyl)-3-((4-chlorophenyl)(2-(trifluoromethyl)phenyl)methoxy)azetidine-1-carboxamide dosed at no more than 200 mg provides a Tmax of 0.1-5, 0.1-4, 0.1-3, 0.1-2, 0.1-1.5, 0.1-1, 0.25-3, 0.25-2, 0.25-1.5, 0.25-1, 0.5-3, 0.5-2.5, 0.5-2, 0.5-1.5, 0.5-1.25, 0.5-1, 0.5-0.75, or 0.1-0.75 hr. In some instances, (R)—N-(tert-butyl)-3-((4-chlorophenyl)(2-(trifluoromethyl)phenyl)methoxy)azetidine-1-carboxamide dosed at no more than 150 mg provides a Tmax of 0.1-5, 0.1-4, 0.1-3, 0.1-2, 0.1-1.5, 0.1-1, 0.25-3, 0.25-2, 0.25-1.5, 0.25-1, 0.5-3, 0.5-2.5, 0.5-2, 0.5-1.5, 0.5-1.25, 0.5-1, 0.5-0.75, or 0.1-0.75 hr. In some instances, (R)—N-(tert-butyl)-3-((4-chlorophenyl)(2-(trifluoromethyl)phenyl)methoxy)azetidine-1-carboxamide dosed at no more than 100 mg provides a Tmax of 0.1-5, 0.1-4, 0.1-3, 0.1-2, 0.1-1.5, 0.1-1, 0.25-3, 0.25-2, 0.25-1.5, 0.25-1, 0.5-3, 0.5-2.5, 0.5-2, 0.5-1.5, 0.5-1.25, 0.5-1, 0.5-0.75, or 0.1-0.75 hr. In some instances, (R)—N-(tert-butyl)-3-((4-chlorophenyl)(2-(trifluoromethyl)phenyl)methoxy)azetidine-1-carboxamide dosed at no more than 75 mg provides a Tmax of 0.1-5, 0.1-4, 0.1-3, 0.1-2, 0.1-1.5, 0.1-1, 0.25-3, 0.25-2, 0.25-1.5, 0.25-1, 0.5-3, 0.5-2.5, 0.5-2, 0.5-1.5, 0.5-1.25, 0.5-1, 0.5-0.75, or 0.1-0.75 hr. In some instances, (R)—N-(tert-butyl)-3-((4-chlorophenyl)(2-(trifluoromethyl)phenyl)methoxy)azetidine-1-carboxamide dosed at no more than 50 mg provides a Tmax of 0.1-5, 0.1-4, 0.1-3, 0.1-2, 0.1-1.5, 0.1-1, 0.25-3, 0.25-2, 0.25-1.5, 0.25-1, 0.5-3, 0.5-2.5, 0.5-2, 0.5-1.5, 0.5-1.25, 0.5-1, 0.5-0.75, or 0.1-0.75 hr. In some instances, (R)—N-(tert-butyl)-3-((4-chlorophenyl)(2-(trifluoromethyl)phenyl)methoxy)azetidine-1-carboxamide dosed at no more than 30 mg provides a Tmax of 0.1-5, 0.1-4, 0.1-3, 0.1-2, 0.1-1.5, 0.1-1, 0.25-3, 0.25-2, 0.25-1.5, 0.25-1, 0.5-3, 0.5-2.5, 0.5-2, 0.5-1.5, 0.5-1.25, 0.5-1, 0.5-0.75, or 0.1-0.75 hr. In some instances, (R)—N-(tert-butyl)-3-((4-chlorophenyl)(2-(trifluoromethyl)phenyl)methoxy)azetidine-1-carboxamide dosed at no more than 20 mg provides a Tmax of 0.1-5, 0.1-4, 0.1-3, 0.1-2, 0.1-1.5, 0.1-1, 0.25-3, 0.25-2, 0.25-1.5, 0.25-1, 0.5-3, 0.5-2.5, 0.5-2, 0.5-1.5, 0.5-1.25, 0.5-1, 0.5-0.75, or 0.1-0.75 hr. In some instances, (R)—N-(tert-butyl)-3-((4-chlorophenyl)(2-(trifluoromethyl)phenyl)methoxy)azetidine-1-carboxamide dosed at 10-50 mg provides a Tmax of 0.1-5, 0.1-4, 0.1-3, 0.1-2, 0.1-1.5, 0.1-1, 0.25-3, 0.25-2, 0.25-1.5, 0.25-1, 0.5-3, 0.5-2.5, 0.5-2, 0.5-1.5, 0.5-1.25, 0.5-1, 0.5-0.75, or 0.1-0.75 hr. In some instances, (R)—N-(tert-butyl)-3-((4-chlorophenyl)(2-(trifluoromethyl)phenyl)methoxy)azetidine-1-carboxamide dosed at 20-75 mg provides a Tmax of 0.1-5, 0.1-4, 0.1-3, 0.1-2, 0.1-1.5, 0.1-1, 0.25-3, 0.25-2, 0.25-1.5, 0.25-1, 0.5-3, 0.5-2.5, 0.5-2, 0.5-1.5, 0.5-1.25, 0.5-1, 0.5-0.75, or 0.1-0.75 hr. In some instances, (R)—N-(tert-butyl)-3-((4-chlorophenyl)(2-(trifluoromethyl)phenyl)methoxy)azetidine-1-carboxamide dosed at 15-50 mg provides a Tmax of 0.1-5, 0.1-4, 0.1-3, 0.1-2, 0.1-1.5, 0.1-1, 0.25-3, 0.25-2, 0.25-1.5, 0.25-1, 0.5-3, 0.5-2.5, 0.5-2, 0.5-1.5, 0.5-1.25, 0.5-1, 0.5-0.75, or 0.1-0.75 hr. In some instances, (R)—N-(tert-butyl)-3-((4-chlorophenyl)(2-(trifluoromethyl)phenyl)methoxy)azetidine-1-carboxamide dosed at 10-100 mg provides a Tmax of 0.1-5, 0.1-4, 0.1-3, 0.1-2, 0.1-1.5, 0.1-1, 0.25-3, 0.25-2, 0.25-1.5, 0.25-1, 0.5-3, 0.5-2.5, 0.5-2, 0.5-1.5, 0.5-1.25, 0.5-1, 0.5-0.75, or 0.1-0.75 hr. In some instances, the dosage is oral.

In some instances, (R)—N-(tert-butyl)-3-((4-chlorophenyl)(2-(trifluoromethyl)phenyl)methoxy)azetidine-1-carboxamide dosed at no more than 200 mg provides a Cmax of about 100-500 ng/mL, about 50-1000 ng/mL, about 50-400 ng/mL, about 50-300 ng/mL, about 50-200 ng/mL, about 50-500 ng/mL, about 75-750 ng/mL, about 100-500 ng/mL, about 100-400 ng/mL, or about 100-300 ng/mL. In some instances, (R)—N-(tert-butyl)-3-((4-chlorophenyl)(2-(trifluoromethyl)phenyl)methoxy)azetidine-1-carboxamide dosed at no more than 100 mg provides a Cmax of about 100-500 ng/mL, about 50-1000 ng/mL, about 50-400 ng/mL, about 50-300 ng/mL, about 50-200 ng/mL, about 50-500 ng/mL, about 75-750 ng/mL, about 100-500 ng/mL, about 100-400 ng/mL, or about 100-300 ng/mL. In some instances, (R)—N-(tert-butyl)-3-((4-chlorophenyl)(2-(trifluoromethyl)phenyl)methoxy)azetidine-1-carboxamide dosed at no more than 75 mg provides a Cmax of about 100-500 ng/mL, about 50-1000 ng/mL, about 50-400 ng/mL, about 50-300 ng/mL, about 50-200 ng/mL, about 50-500 ng/mL, about 75-750 ng/mL, about 100-500 ng/mL, about 100-400 ng/mL, or about 100-300 ng/mL. In some instances, (R)—N-(tert-butyl)-3-((4-chlorophenyl)(2-(trifluoromethyl)phenyl)methoxy)azetidine-1-carboxamide dosed at no more than 50 mg provides a Cmax of about 100-500 ng/mL, about 50-1000 ng/mL, about 50-400 ng/mL, about 50-300 ng/mL, about 50-200 ng/mL, about 50-500 ng/mL, about 75-750 ng/mL, about 100-500 ng/mL, about 100-400 ng/mL, or about 100-300 ng/mL. In some instances, (R)—N-(tert-butyl)-3-((4-chlorophenyl)(2-(trifluoromethyl)phenyl)methoxy)azetidine-1-carboxamide dosed at no more than 30 mg provides a Cmax of about 100-500 ng/mL, about 50-1000 ng/mL, about 50-400 ng/mL, about 50-300 ng/mL, about 50-200 ng/mL, about 50-500 ng/mL, about 75-750 ng/mL, about 100-500 ng/mL, about 100-400 ng/mL, or about 100-300 ng/mL. In some instances, (R)—N-(tert-butyl)-3-((4-chlorophenyl)(2-(trifluoromethyl)phenyl)methoxy)azetidine-1-carboxamide dosed at no more than 20 mg provides a Cmax of about 100-500 ng/mL, about 50-1000 ng/mL, about 50-400 ng/mL, about 50-300 ng/mL, about 50-200 ng/mL, about 50-500 ng/mL, about 75-750 ng/mL, about 100-500 ng/mL, about 100-400 ng/mL, or about 100-300 ng/mL. In some instances, (R)—N-(tert-butyl)-3-((4-chlorophenyl)(2-(trifluoromethyl)phenyl)methoxy)azetidine-1-carboxamide dosed at no more than 15 mg provides a Cmax of about 100-500 ng/mL, about 50-1000 ng/mL, about 50-400 ng/mL, about 50-300 ng/mL, about 50-200 ng/mL, about 50-500 ng/mL, about 75-750 ng/mL, about 100-500 ng/mL, about 100-400 ng/mL, or about 100-300 ng/mL. In some instances, (R)—N-(tert-butyl)-3-((4-chlorophenyl)(2-(trifluoromethyl)phenyl)methoxy)azetidine-1-carboxamide dosed at no more than 10 mg provides a Cmax of about 100-500 ng/mL, about 50-1000 ng/mL, about 50-400 ng/mL, about 50-300 ng/mL, about 50-200 ng/mL, about 50-500 ng/mL, about 75-750 ng/mL, about 100-500 ng/mL, about 100-400 ng/mL, or about 100-300 ng/mL. In some instances, (R)—N-(tert-butyl)-3-((4-chlorophenyl)(2-(trifluoromethyl)phenyl)methoxy)azetidine-1-carboxamide dosed at no more than 5 mg provides a Cmax of about 100-500 ng/mL, about 50-1000 ng/mL, about 50-400 ng/mL, about 50-300 ng/mL, about 50-200 ng/mL, about 50-500 ng/mL, about 75-750 ng/mL, about 100-500 ng/mL, about 100-400 ng/mL, or about 100-300 ng/mL. In some instances, (R)—N-(tert-butyl)-3-((4-chlorophenyl)(2-(trifluoromethyl)phenyl)methoxy)azetidine-1-carboxamide dosed at no more than 1 mg provides a Cmax of about 100-500 ng/mL, about 50-1000 ng/mL, about 50-400 ng/mL, about 50-300 ng/mL, about 50-200 ng/mL, about 50-500 ng/mL, about 75-750 ng/mL, about 100-500 ng/mL, about 100-400 ng/mL, or about 100-300 ng/mL. In some instances, (R)—N-(tert-butyl)-3-((4-chlorophenyl)(2-(trifluoromethyl)phenyl)methoxy)azetidine-1-carboxamide dosed at 15-50 mg provides a Cmax of about 100-500 ng/mL, about 50-1000 ng/mL, about 50-400 ng/mL, about 50-300 ng/mL, about 50-200 ng/mL, about 50-500 ng/mL, about 75-750 ng/mL, about 100-500 ng/mL, about 100-400 ng/mL, or about 100-300 ng/mL. In some instances, (R)—N-(tert-butyl)-3-((4-chlorophenyl)(2-(trifluoromethyl)phenyl)methoxy)azetidine-1-carboxamide dosed at 20-50 mg provides a Cmax of about 100-500 ng/mL, about 50-1000 ng/mL, about 50-400 ng/mL, about 50-300 ng/mL, about 50-200 ng/mL, about 50-500 ng/mL, about 75-750 ng/mL, about 100-500 ng/mL, about 100-400 ng/mL, or about 100-300 ng/mL. In some instances, (R)—N-(tert-butyl)-3-((4-chlorophenyl)(2-(trifluoromethyl)phenyl)methoxy)azetidine-1-carboxamide dosed at 10-75 mg provides a Cmax of about 100-500 ng/mL, about 50-1000 ng/mL, about 50-400 ng/mL, about 50-300 ng/mL, about 50-200 ng/mL, about 50-500 ng/mL, about 75-750 ng/mL, about 100-500 ng/mL, about 100-400 ng/mL, or about 100-300 ng/mL. In some instances, the dosage is oral.

In some instances, (R)—N-(tert-butyl)-3-((4-chlorophenyl)(2-(trifluoromethyl)phenyl)methoxy)azetidine-1-carboxamide provides a dosage normalized plasma AUCₗₐₛₜ of about 25-600 h*ng/mL/mg, about 25-500 h*ng/mL/mg, about 25-450 h*ng/mL/mg, about 50-500 h*ng/mL/mg, about 50-450 h*ng/mL/mg, about 50-400 h*ng/mL/mg, about 50-300 h*ng/mL/mg, about 50-250 h*ng/mL/mg, about 40-300 h*ng/mL/mg, or about 50-200 h*ng/mL/mg. In some instances, (R)—N-(tert-butyl)-3-((4-chlorophenyl)(2-(trifluoromethyl)phenyl)methoxy)azetidine-1-carboxamide dosed at no more than 200 mg provides a AUCₗₐₛₜ of about 1000-3500 h*ng/mL, about 1000-3000 ng/mL, about 1250-3000 ng/mL, about 1500-2800 ng/mL, about 1000-2500 ng/mL, or about 1200-3000 ng/mL. In some instances, (R)—N-(tert-butyl)-3-((4-chlorophenyl)(2-(trifluoromethyl)phenyl)methoxy)azetidine-1-carboxamide dosed at no more than 150 mg provides a AUCₗₐₛₜ of about 1000-3500 h*ng/mL, about 1000-3000 ng/mL, about 1250-3000 ng/mL, about 1500-2800 ng/mL, about 1000-2500 ng/mL, or about 1200-3000 ng/mL. In some instances, (R)—N-(tert-butyl)-3-((4-chlorophenyl)(2-(trifluoromethyl)phenyl)methoxy)azetidine-1-carboxamide dosed at no more than 100 mg provides a AUCₗₐₛₜ of about 1000-3500 h*ng/mL, about 1000-3000 ng/mL, about 1250-3000 ng/mL, about 1500-2800 ng/mL, about 1000-2500 ng/mL, or about 1200-3000 ng/mL. In some instances, (R)—N-(tert-butyl)-3-((4-chlorophenyl)(2-(trifluoromethyl)phenyl)methoxy)azetidine-1-carboxamide dosed at no more than 75 mg provides a AUCₗₐₛₜ of about 1000-3500 h*ng/mL, about 1000-3000 ng/mL, about 1250-3000 ng/mL, about 1500-2800 ng/mL, about 1000-2500 ng/mL, or about 1200-3000 ng/mL. In some instances, (R)—N-(tert-butyl)-3-((4-chlorophenyl)(2-(trifluoromethyl)phenyl)methoxy)azetidine-1-carboxamide dosed at no more than 50 mg provides a AUCₗₐₛₜ of about 1000-3500 h*ng/mL, about 1000-3000 ng/mL, about 1250-3000 ng/mL, about 1500-2800 ng/mL, about 1000-2500 ng/mL, or about 1200-3000 ng/mL. In some instances, (R)—N-(tert-butyl)-3-((4-chlorophenyl)(2-(trifluoromethyl)phenyl)methoxy)azetidine-1-carboxamide dosed at no more than 40 mg provides a AUCₗₐₛₜ of about 1000-3500 h*ng/mL, about 1000-3000 ng/mL, about 1250-3000 ng/mL, about 1500-2800 ng/mL, about 1000-2500 ng/mL, or about 1200-3000 ng/mL. In some instances, (R)—N-(tert-butyl)-3-((4-chlorophenyl)(2-(trifluoromethyl)phenyl)methoxy)azetidine-1-carboxamide dosed at no more than 30 mg provides a AUCₗₐₛₜ of about 1000-3500 h*ng/mL, about 1000-3000 ng/mL, about 1250-3000 ng/mL, about 1500-2800 ng/mL, about 1000-2500 ng/mL, or about 1200-3000 ng/mL. In some instances, (R)—N-(tert-butyl)-3-((4-chlorophenyl)(2-(trifluoromethyl)phenyl)methoxy)azetidine-1-carboxamide dosed at no more than 20 mg provides a AUCₗₐₛₜ of about 1000-3500 h*ng/mL, about 1000-3000 ng/mL, about 1250-3000 ng/mL, about 1500-2800 ng/mL, about 1000-2500 ng/mL, or about 1200-3000 ng/mL. In some instances, (R)—N-(tert-butyl)-3-((4-chlorophenyl)(2-(trifluoromethyl)phenyl)methoxy)azetidine-1-carboxamide dosed at no more than 15 mg provides a AUCₗₐₛₜ of about 1000-3500 h*ng/mL, about 1000-3000 ng/mL, about 1250-3000 ng/mL, about 1500-2800 ng/mL, about 1000-2500 ng/mL, or about 1200-3000 ng/mL. In some instances, (R)—N-(tert-butyl)-3-((4-chlorophenyl)(2-(trifluoromethyl)phenyl)methoxy)azetidine-1-carboxamide dosed at no more than 10 mg provides a AUCₗₐₛₜ of about 500-3500 h*ng/mL, about 500-3000 ng/mL, about 1250-3000 ng/mL, about 1500-2800 ng/mL, about 1000-2500 ng/mL, or about 1200-3000 ng/mL. In some instances, (R)—N-(tert-butyl)-3-((4-chlorophenyl)(2-(trifluoromethyl)phenyl)methoxy)azetidine-1-carboxamide dosed at no more than 5 mg provides a AUCₗₐₛₜ of about 100-3500 h*ng/mL, about 200-3000 ng/mL, about 1250-3000 ng/mL, about 500-2800 ng/mL, about 300-2500 ng/mL, or about 400-3000 ng/mL. In some instances, (R)—N-(tert-butyl)-3-((4-chlorophenyl)(2-(trifluoromethyl)phenyl)methoxy)azetidine-1-carboxamide dosed at no more than 1 mg provides a AUCₗₐₛₜ of about 100-3500 h*ng/mL, about 100-3000 ng/mL, about 125-3000 ng/mL, about 150-2800 ng/mL, about 200-2500 ng/mL, or about 500-3000 ng/mL. In some instances, the dosage is oral.

In some instances, (R)—N-(tert-butyl)-3-((4-chlorophenyl)(2-(trifluoromethyl)phenyl)methoxy)azetidine-1-carboxamide provides a dosage normalized plasma AUC₅h of about 5-60 h*ng/mL/mg, about 5-50 h*ng/mL/mg, about 5-45 h*ng/mL/mg, about 5-45 h*ng/mL/mg, about 7-60 h*ng/mL/mg, about 7-50 h*ng/mL/mg, about 7-45 h*ng/mL/mg, about 7-40 h*ng/mL/mg, about 10-60 h*ng/mL/mg, about 10-50 h*ng/mL/mg, about 10-50 h*ng/mL/mg, or about 10-40 h*ng/mL/mg. In some instances, (R)—N-(tert-butyl)-3-((4-chlorophenyl)(2-(trifluoromethyl)phenyl)methoxy)azetidine-1-carboxamide dosed at no more than 200 mg provides a plasma AUC₅ₕ of about 200-2000 h*ng/mL, about 200-1500 h*ng/mL, about 200-1200 h*ng/mL, about 300-2500 h*ng/mL, about 300-2000 h*ng/mL, about 300-1750 h*ng/mL, about 300-1500 h*ng/mL, about 400-3000 h*ng/mL, about 400-2000 h*ng/mL, about 400-1500 h*ng/mL, about 400-1200 h*ng/mL, or about 400-1000 h*ng/mL. In some instances, (R)—N-(tert-butyl)-3-((4-chlorophenyl)(2-(trifluoromethyl)phenyl)methoxy)azetidine-1-carboxamide dosed at no more than 150 mg provides a plasma AUC₅ₕ of about 200-2000 h*ng/mL, about 200-1500 h*ng/mL, about 200-1200 h*ng/mL, about 300-2500 h*ng/mL, about 300-2000 h*ng/mL, about 300-1750 h*ng/mL, about 300-1500 h*ng/mL, about 400-3000 h*ng/mL, about 400-2000 h*ng/mL, about 400-1500 h*ng/mL, about 400-1200 h*ng/mL, or about 400-1000 h*ng/mL. In some instances, (R)—N-(tert-butyl)-3-((4-chlorophenyl)(2-(trifluoromethyl)phenyl)methoxy)azetidine-1-carboxamide dosed at no more than 100 mg provides a plasma AUC₅ₕ of about 200-2000 h*ng/mL, about 200-1500 h*ng/mL, about 200-1200 h*ng/mL, about 300-2500 h*ng/mL, about 300-2000 h*ng/mL, about 300-1750h*ng/mL, about 300-1500 h*ng/mL, about 400-3000 h*ng/mL, about 400-2000 h*ng/mL, about 400-1500 h*ng/mL, about 400-1200 h*ng/mL, or about 400-1000 h*ng/mL. In some instances, (R)—N-(tert-butyl)-3-((4-chlorophenyl)(2-(trifluoromethyl)phenyl)methoxy)azetidine-1-carboxamide dosed at no more than 75 mg provides a plasma AUC₅ₕ of about 200-2000 h*ng/mL, about 200-1500 h*ng/mL, about 200-1200 h*ng/mL, about 300-2500 h*ng/mL, about 300-2000 h*ng/mL, about 300-1750 h*ng/mL, about 300-1500 h*ng/mL, about 400-3000 h*ng/mL, about 400-2000 h*ng/mL, about 400-1500 h*ng/mL, about 400-1200 h*ng/mL, or about 400-1000 h*ng/mL. In some instances, (R)—N-(tert-butyl)-3-((4-chlorophenyl)(2-(trifluoromethyl)phenyl)methoxy)azetidine-1-carboxamide dosed at no more than 50 mg provides a plasma AUC₅ₕ of about 200-2000 h*ng/mL, about 200-1500 h*ng/mL, about 200-1200 h*ng/mL, about 300-2500 h*ng/mL, about 300-2000 h*ng/mL, about 300-1750 h*ng/mL, about 300-1500 h*ng/mL, about 400-3000 h*ng/mL, about 400-2000 h*ng/mL, about 400-1500 h*ng/mL, about 400-1200 h*ng/mL, or about 400-1000 h*ng/mL. In some instances, (R)—N-(tert-butyl)-3-((4-chlorophenyl)(2-(trifluoromethyl)phenyl)methoxy)azetidine-1-carboxamide dosed at no more than 40 mg provides a plasma AUC₅ₕ of about 200-2000 h*ng/mL, about 200-1500 h*ng/mL, about 200-1200 h*ng/mL, about 300-2500 h*ng/mL, about 300-2000 h*ng/mL, about 300-1750 h*ng/mL, about 300-1500 h*ng/mL, about 400-3000 h*ng/mL, about 400-2000 h*ng/mL, about 400-1500 h*ng/mL, about 400-1200 h*ng/mL, or about 400-1000 h*ng/mL. In some instances, (R)—N-(tert-butyl)-3-((4-chlorophenyl)(2-(trifluoromethyl)phenyl)methoxy)azetidine-1-carboxamide dosed at no more than 30 mg provides a plasma AUC₅h of about 200-2000 h*ng/mL, about 200-1500 h*ng/mL, about 200-1200 h*ng/mL, about 300-2500 h*ng/mL, about 300-2000 h*ng/mL, about 300-1750 h*ng/mL, about 300-1500h*ng/mL, about 400-3000h*ng/mL, about 400-2000 h*ng/mL, about 400-1500 h*ng/mL, about 400-1200 h*ng/mL, or about 400-1000 h*ng/mL. In some instances, (R)—N-(tert-butyl)-3-((4-chlorophenyl)(2-(trifluoromethyl)phenyl)methoxy)azetidine-1-carboxamide dosed at no more than 25 mg provides a plasma AUC₅ₕ of about 200-2000 h*ng/mL, about 200-1500 h*ng/mL, about 200-1200 h*ng/mL, about 300-2500 h*ng/mL, about 300-2000 h*ng/mL, about 300-1750 h*ng/mL, about 300-1500 h*ng/mL, about 400-3000 h*ng/mL, about 400-2000 h*ng/mL, about 400-1500 h*ng/mL, about 400-1200 h*ng/mL, or about 400-1000 h*ng/mL. In some instances, (R)—N-(tert-butyl)-3-((4-chlorophenyl)(2-(trifluoromethyl)phenyl)methoxy)azetidine-1-carboxamide dosed at no more than 20 mg provides a plasma AUC₅h of about 200-2000 h*ng/mL, about 200-1500 h*ng/mL, about 200-1200 h*ng/mL, about 300-2500 h*ng/mL, about 300-2000 h*ng/mL, about 300-1750 h*ng/mL, about 300-1500 h*ng/mL, about 400-3000 h*ng/mL, about 400-2000 h*ng/mL, about 400-1500 h*ng/mL, about 400-1200 h*ng/mL, or about 400-1000 h*ng/mL. In some instances, (R)—N-(tert-butyl)-3-((4-chlorophenyl)(2-(trifluoromethyl)phenyl)methoxy)azetidine-1-carboxamide dosed at no more than 15 mg provides a plasma AUC₅ₕ of about 200-2000 h*ng/mL, about 200-1500 h*ng/mL, about 200-1200 h*ng/mL, about 300-2500 h*ng/mL, about 300-2000 h*ng/mL, about 300-1750 h*ng/mL, about 300-1500 h*ng/mL, about 400-3000 h*ng/mL, about 400-2000 h*ng/mL, about 400-1500 h*ng/mL, about 400-1200 h*ng/mL, or about 400-1000 h*ng/mL. In some instances, (R)—N-(tert-butyl)-3-((4-chlorophenyl)(2-(trifluoromethyl)phenyl)methoxy)azetidine-1-carboxamide dosed at no more than 10 mg provides a plasma AUC₅h of about 200-2000 h*ng/mL, about 200-1500 h*ng/mL, about 200-1200 h*ng/mL, about 300-2500 h*ng/mL, about 300-2000 h*ng/mL, about 300-1750 h*ng/mL, about 300-1500 h*ng/mL, about 400-3000 h*ng/mL, about 400-2000 h*ng/mL, about 400-1500 h*ng/mL, about 400-1200 h*ng/mL, or about 400-1000 h*ng/mL. In some instances, (R)—N-(tert-butyl)-3-((4-chlorophenyl)(2-(trifluoromethyl)phenyl)methoxy)azetidine-1-carboxamide dosed at no more than 5 mg provides a plasma AUC₅ₕ of about 200-2000 h*ng/mL, about 200-1500 h*ng/mL, about 200-1200 h*ng/mL, about 300-2500 h*ng/mL, about 300-2000 h*ng/mL, about 300-1750 h*ng/mL, about 300-1500 h*ng/mL, about 400-3000 h*ng/mL, about 400-2000 h*ng/mL, about 400-1500 h*ng/mL, about 400-1200 h*ng/mL, or about 400-1000 h*ng/mL. In some instances, (R)—N-(tert-butyl)-3-((4-chlorophenyl)(2-(trifluoromethyl)phenyl)methoxy)azetidine-1-carboxamide dosed at no more than 1 mg provides a plasma AUC₅ₕ of about 200-2000 h*ng/mL, about 200-1500 h*ng/mL, about 200-1200 h*ng/mL, about 300-2500 h*ng/mL, about 300-2000 h*ng/mL, about 300-1750 h*ng/mL, about 300-1500 h*ng/mL, about 400-3000 h*ng/mL, about 400-2000 h*ng/mL, about 400-1500 h*ng/mL, about 400-1200 h*ng/mL, or about 400-1000 h*ng/mL. In some instances, the dosage is oral.

In various embodiments, the method reduces the subject's ability to experience euphoria after inhaling or consuming Cannabis or a synthetic cannabinoid.

Compositions and formulations described herein may be administered as single or multiple doses. In some embodiments, described herein are methods of using the (R)—N-(tert-butyl)-3-((4-chlorophenyl)(2-(trifluoromethyl)phenyl)methoxy)azetidine-1-carboxamide as a single dose, one-time treatment for overdose THC or SC, or both. The overdose can also be from consumption of Cannabis, synthetic cannabinoid, or any compound that is an agonist of the CB1 receptor. In some instances, methods described herein include treatment to children who inadvertently consume Cannabis or cannabinoid edibles. In related aspects, any suspected overdose patient that presents a mentally disoriented or psychotic or cannot articulate the nature of their condition or the substances that have been ingested or administered can be treated with (R)—N-(tert-butyl)-3-((4-chlorophenyl)(2-(trifluoromethyl)phenyl)methoxy)azetidine-1-carboxamide.

The methods described herein include pre-exposure prophylaxis treatments. The long-term effects of CB1 antagonism, which in some instances includes anhedonia, potentially makes them unsuitable for chronic use. However, in the same way that a subject who is addicted to alcohol might consume disulfiram before entering a situation when tempted to consume alcohol, one can take a CB1 antagonist, such as (R)—N-(tert-butyl)-3-((4-chlorophenyl)(2-(trifluoromethyl)phenyl)methoxy)azetidine-1-carboxamide, before encountering a situation where they may likely be exposed to or tempted to ingest THC or SCs or both. Similarly, in some instances, (R)—N-(tert-butyl)-3-((4-chlorophenyl)(2-(trifluoromethyl)phenyl)methoxy)azetidine-1-carboxamide is used to prevent effects from secondhand smoke from marijuana. The method of using (R)—N-(tert-butyl)-3-((4-chlorophenyl)(2-(trifluoromethyl)phenyl)methoxy)azetidine-1-carboxamide in some instances includes use by a subject who wishes to gain acceptance to a situation or group by smoking marijuana or SCs, but also wants to remain mentally alert, such as during an undercover police or law enforcement investigation.

### Additional Definitions

As used herein, “active agent” is used to indicate a chemical entity which has biological activity. In certain embodiments, an “active agent” is a compound having pharmaceutical utility. For example an active agent may be an anti-cancer therapeutic.

As used herein, “modulation” refers to a change in activity as a direct or indirect response to the presence of a chemical entity as described herein, relative to the activity of in the absence of the chemical entity. The change may be an increase in activity or a decrease in activity, and may be due to the direct interaction of the compound with the target or due to the interaction of the compound with one or more other factors that in turn affect the target's activity. For example, the presence of the chemical entity may, for example, increase or decrease the target activity by directly binding to the target, by causing (directly or indirectly) another factor to increase or decrease the target activity, or by (directly or indirectly) increasing or decreasing the amount of target present in the cell or organism.

As used herein, “therapeutically effective amount” of a chemical entity described herein refers to an amount effective, when administered to a human or non-human subject, to provide a therapeutic benefit such as amelioration of symptoms, slowing of disease progression, or prevention of disease.

“Treating” or “treatment” encompasses administration of Compound 1 or metabolite thereof, or a pharmaceutically acceptable salt thereof, to a mammalian subject, particularly a human subject, in need of such an administration and includes (i) arresting the development of clinical symptoms of the disease, such as cancer, (ii) bringing about a regression in the clinical symptoms of the disease, such as cancer, and/or (iii) prophylactic treatment for preventing the onset of the disease, such as cancer.

As used herein, a “pharmaceutically acceptable” component is one that is suitable for use with humans and/or animals without undue adverse side effects (such as toxicity, irritation, and allergic response) commensurate with a reasonable benefit/risk ratio.

“Pharmaceutically acceptable salts” include, but are not limited to salts with inorganic acids, such as hydrochlorate, carbonate, phosphate, hydrogenphosphate, diphosphate, hydrobromate, sulfate, sulfinate, nitrate, and like salts; as well as salts with an organic acid, such as malate, malonate, maleate, fumarate, tartrate, succinate, citrate, acetate, lactate, gluconate, methanesulfonate, Tris (hydroxymethyl-aminomethane), p-toluenesulfonate, propionate, 2-hydroxyethylsulfonate, benzoate, salicylate, stearate, oxalate, pamoate, and alkanoate such as acetate, HOOC—(CH₂)ₙ—COOH where n is 0-4, and like salts. Other salts include sulfate, methanesulfonate, bromide, trifluoroacetate, picrate, sorbate, benzilate, salicylate, nitrate, phthalate or morpholine. Pharmaceutically acceptable cations include, but are not limited to sodium, potassium, calcium, aluminum, lithium, and ammonium.

In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

As used herein, “subject” refers to a mammal that has been or will be the object of treatment, observation or experiment. The methods described herein can be useful in both human therapy and veterinary applications. In some embodiments, the subject is a human.

“Prodrugs” described herein include any compound that becomes Compound 1 or metabolite thereof when administered to a subject, e.g., upon metabolic processing of the prodrug. Similarly, “pharmaceutically acceptable salts” includes “prodrugs” of pharmaceutically acceptable salts. Examples of prodrugs include derivatives of functional groups, such as a carboxylic acid group, in Compound 1 or metabolite thereof. Exemplary prodrugs of a carboxylic acid group include, but are not limited to, carboxylic acid esters such as alkyl esters, hydroxyalkyl esters, arylalkyl esters, and aryloxyalkyl esters. Other exemplary prodrugs include lower alkyl esters such as ethyl ester, acyloxyalkyl esters such as pivaloyloxymethyl (POM), glycosides, and ascorbic acid derivatives. Other exemplary prodrugs include amides of carboxylic acids. A discussion of prodrugs is provided in T. Higuchi and V. Stella, Pro-drugs as Novel Delivery Systems, Vol. 14 of the A.C.S. Symposium Series, in Edward B. Roche, ed., Bioreversible Carriers in Drug Design, American Pharmaceutical Association and Pergamon Press, 1987, and in Design of Prodrugs, ed. H. Bundgaard, Elsevier, 1985.

A “solvate” is formed by the interaction of a solvent and a compound. The term “compound” is intended to include solvates of compounds. Similarly, “pharmaceutically acceptable salts” includes solvates of pharmaceutically acceptable salts. Suitable solvates are pharmaceutically acceptable solvates, such as hydrates, including monohydrates and hemi-hydrates. Also included are solvates formed with the one or more crystallization solvents.

Pharmaceutically acceptable forms of the compounds recited herein include pharmaceutically acceptable salts, chelates, non-covalent complexes, prodrugs, and mixtures thereof.

A “chelate” is formed by the coordination of a compound to a metal ion at two (or more) points. The term “compound” is intended to include chelates of compounds. Similarly, “pharmaceutically acceptable salts” includes chelates of pharmaceutically acceptable salts.

A “non-covalent complex” is formed by the interaction of a compound and another molecule wherein a covalent bond is not formed between the compound and the molecule. For example, complexation can occur through van der Waals interactions, hydrogen bonding, and electrostatic interactions (also called ionic bonding). Such non-covalent complexes are included in the term “compound”. Similarly, pharmaceutically acceptable salts include “non-covalent complexes” of pharmaceutically acceptable salts.

A “healthcare provider” can be a physician, nurse, physician assistant, pharmacist, or similarly trained personnel. An “emergency responder” can be an EMT (emergency medical technician), fireman, police officer, security officer, community outreach office, or other first responder.

When ranges are used herein for physical properties, such as molecular weight, or chemical properties, such as chemical formulae, all combinations and sub combinations of ranges and specific embodiments therein are intended to be included.

The term “about” when referring to a number or a numerical range means that the number or numerical range referred to is an approximation within experimental variability (or within statistical experimental error), and thus the number or numerical range may vary from, for example, between 1% and 15% of the stated number or numerical range. In some instances of numerical ranges, “about” means±10%.

As used herein, “significant” refers to any detectable change that is statistically significant in a standard parametric test of statistical significance such as Student's T-test, where p<0.05.

As used herein, “level” means the levels of Glasgow coma metric or coma scale.

### EXAMPLES

The following examples serve to further describe the manner of using the present disclosure. These examples are presented for illustrative purpose and should not serve to limit the true scope of the present disclosure.

In carrying out the procedures of the methods described herein, it is of course to be understood that references to particular buffers, media, reagents, cells, culture conditions, and the like are not intended to be limiting, but are to be read so as to include all related materials that one of ordinary skill in the art would recognize as being of interest or value in the particular context in which that discussion is presented. For example, it is often possible to substitute on buffer system or culture medium for another and still achieve similar, if not identical, results. Those of skill in the art will have sufficient knowledge of such systems and methodologies so as to able, without undue experimentation, to make such substitutions as will optimally serve their purposes in using the methods and procedures disclosed herein.

### Example 1—PD Evaluation of 50 mg and 100 mg Oral Dosage of Compound 1 Design

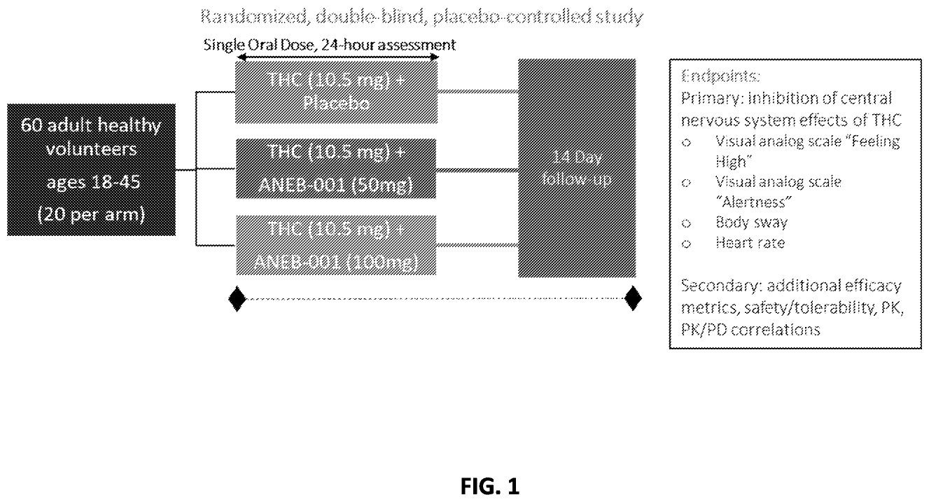

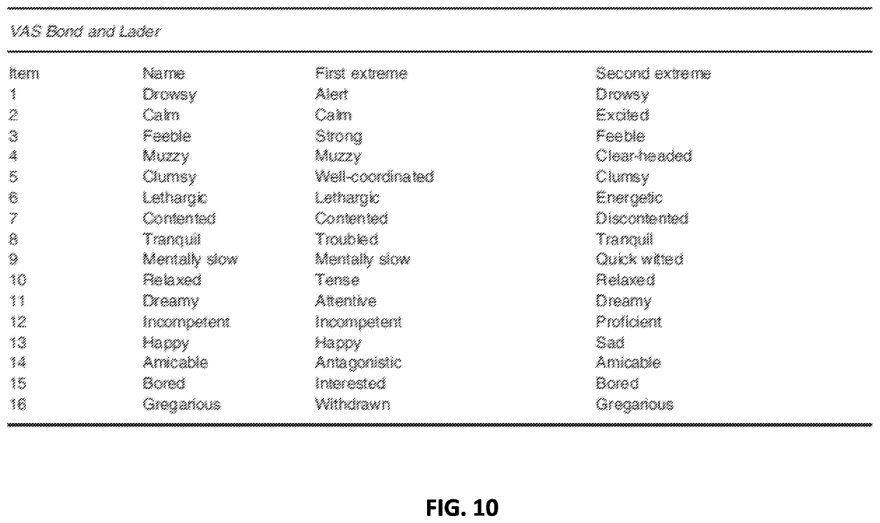

A phase 2 clinical study was conducted using the design outlined in FIG. 1. Briefly, 60 adult healthy volunteers ages 18-45 (20 per arm) were selected for a randomized, double blind placebo-controlled study. Volunteers were divided into three groups: (1) THC (Δ9-Tetrahydrocannabinol, 10.5 mg)+placebo; (2) THC (10.5 mg)+Compound 1 (50 mg); and (3) THC (10.5 mg)+Compound 1 (100 mg). Volunteers received single oral dosages of Compound 1 or placebo, and were observed for 24 hours. A followup evaluation was also performed after 14 days. Primary endpoints included inhibition of central nervous system effects of THC using (a) visual analog scale “feeling high”; (b) visual analog scale “alertness”; (c) body sway; (d) heart rate. Secondary endpoints included additional efficacy metrics, safety/tolerability, PK, and PK/PD correlations. Assessments are shown in FIG. 10.

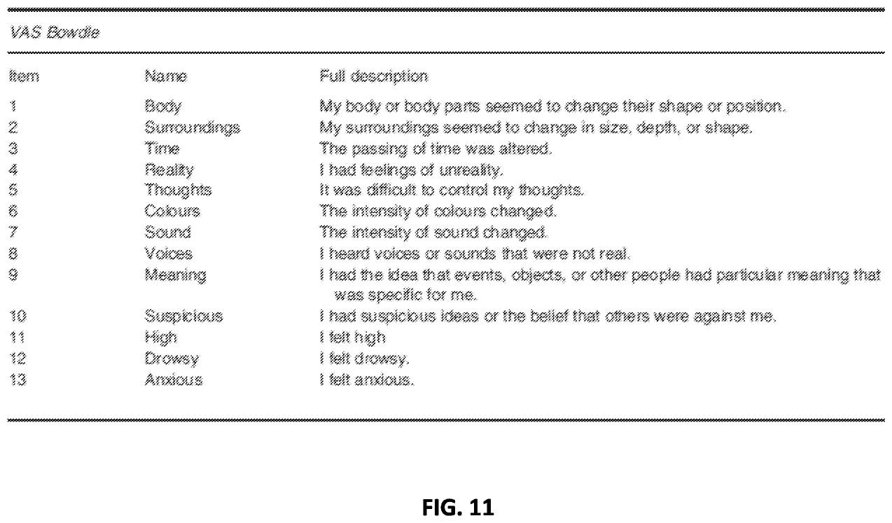

VAS assessments for subjective feelings were carried out according to the general methods of Bond, A. and Lader, M. The use of analogue scales in rating subjective feelings. Br J Med Psychol. 1974; 47:211-218. doi: 10.1111/j.2044-8341.1974.tb02285.x, FIG. 10 and psychomimetic effects using the general methods of Bowdle scales (Bowdle, T. A., et al. (1998) Psychedelic effects of ketamine in healthy volunteers: relationship to steady-state plasma concentrations. Anesthesiology, 88(1), 82-88. DOI: 10.1038/sj.npp.1395214, FIG. 11.

The N-back working memory test was conducted according to the general methods of Romboutset, et al.,: The N-Back test consists of three conditions, with increased working memory load: (Condition 0) “X” condition, in which participants are required to indicate whether the presented letter is a “X” (=target) or another letter; In Condition 1 and 2, letters will be presented sequentially (1.5 seconds for a letter [consonant, except for the letter “z”] presentation, followed by a black screen for 0.5 seconds). “z” is pressed on the keyboard for a target and “/” is pressed for a non-target. Condition 1, “1-back” condition, in which participants are required to indicate whether the letter presented is a repetition without any other letter intervening (e.g., B . . . B); Condition 2, “2-back” condition, in which participants are required to indicate whether a letter is repeated with one other letter in between (e.g., B . . . C . . . B). The 3 conditions are presented in 3 blocks with increasing working memory load. Each condition starts with a training (7 consonants; target:non-target3:4), followed by the test (24 consonants; target:non-target1:3). This test takes approximately 10 minutes.

### Inclusion Criteria.

1. Signed informed consent prior to any study-mandated procedure.
2. Healthy male or female subjects, 18 to 45 years of age, inclusive at screening.
3. Body mass index (BMI) between 18 and 30 kg/m², inclusive at screening, and with a minimum weight of 50 kg.
4. All women of childbearing potential and all males must practice effective contraception during the study and be willing and able to continue contraception for at least 90 days after their last dose of study treatment.
5. Has the ability to communicate well with the Investigator in the Dutch language and willing to comply with the study restrictions.
6. Occasional Cannabis users with (a) Lifetime Cannabis use on at least 10 occasions. (b) In the past 6 months, the mean Cannabis use should not exceed one occasion per week. (c) Subjects should be able to refrain from using cannabinoids at least 3 weeks prior to dosing up to the end of the study. (d) Urine drug screen must be negative prior to dosing.

### Exclusion Criteria.

1. Evidence of any active or chronic disease or condition that could interfere with, or for which the treatment of might interfere with, the conduct of the study, or that would pose an unacceptable risk to the subject in the opinion of the investigator (following a detailed medical history, physical examination, vital signs (systolic and diastolic blood pressure, pulse rate, body temperature) and 12-lead electrocardiogram (ECG)). Minor deviations from the normal range may be accepted, if judged by the Investigator to have no clinical relevance.
2. Clinically significant abnormalities, as judged by the investigator, in laboratory test results (including hepatic and renal panels, complete blood count, chemistry panel and urinalysis). In the case of uncertain or questionable results, tests performed during screening may be repeated before randomization to confirm eligibility or judged to be clinically irrelevant for healthy subjects.
3. Positive Hepatitis B surface antigen (HBsAg), Hepatitis C antibody (HCV Ab), or human immunodeficiency virus antibody (HIV Ab) at screening.
4. Systolic blood pressure (SBP) greater than 140 or less than 90 mm Hg, and diastolic blood pressure (DBP) greater than 95 or less than 50 mm Hg at screening.
5. Abnormal findings in the resting ECG at screening defined as: (a) QTcF>450 or <300 msec for men and QTcF>470 or <300 msec for women (b) Notable resting bradycardia (HR<40 bpm) or tachycardia (HR>100 bpm) (c) Personal or family history of congenital long QT syndrome or sudden death; (d) ECG with QRS and/or T wave judged to be unfavorable for a consistently accurate QT measurement (e.g., neuromuscular artefact that cannot be readily eliminated, arrhythmias, indistinct QRS onset, low amplitude T wave, merged T- and U-waves, prominent U waves); (e) Evidence of atrial fibrillation, atrial flutter, complete branch block, Wolf-Parkinson-White Syndrome, or cardiac pacemaker.
6. Use of any medications (prescription or over-the-counter [OTC]), within 7 days of study drug administration, or less than 5 half-lives (whichever is longer). Exceptions are paracetamol (up to 4 g/day) and ibuprofen (up to Ig/day) and topical medications that are not expected to reach a meaningful systemic exposure, as judged by the investigator. Other exceptions will only be made if the rationale is clearly documented by the investigator.
7. Use of any vitamin, mineral, herbal, and dietary supplements within 7 days of study drug administration, or less than 5 half-lives (whichever is longer). Exceptions will only be made if the rationale is clearly documented by the investigator.
8. Participation in an investigational drug or device study (last dosing of previous study was within 90 days prior to first dosing of this study).
9. History of abuse of addictive substances (alcohol, illegal substances) or current use of more than 21 units alcohol per week, drug abuse, or regular user of sedatives, hypnotics, tranquillizers, or any other addictive agent other than recreative use of THC.
10. Positive test for drugs of abuse (other than THC) at screening.
11. Positive test for drugs of abuse pre-dose.
12. Alcohol will not be allowed from at least 24 hours before screening or pre-dose.
13. Smoker of more than 10 cigarettes per day prior to screening or who use tobacco products equivalent to more than 10 cigarettes per day and unable to abstain from smoking whilst in the unit.
14. Is demonstrating excess in caffeine consumption (more than eight cups of coffee or equivalent per day.
15. Any confirmed significant allergic reactions (urticaria or anaphylaxis) against any drug, or multiple drug allergies (non-active hay fever is acceptable).
16. Loss or donation of blood over 500 mL within three months (males) or four months (females) prior to screening or intention to donate blood or blood products during the study.
17. If a woman: pregnant, or breast-feeding, or planning to become pregnant during the study.
18. Any known factor, condition, or disease that might interfere with treatment compliance, study conduct or interpretation of the results such as drug or alcohol dependence or psychiatric disease.
19. Clinically significant suicidal ideation in the past 5 years as judged by the investigator or any life-time suicide attempts.
20. History of Cannabis-induced psychosis, schizophrenia or other clinically relevant psychiatric disorders, as judged by the investigator.
21. History of a clinically significant mood disorder, including but not limited to major depressive disorder, as judged by the investigator.

### Results

Endpoints were evaluated and are summarized below.

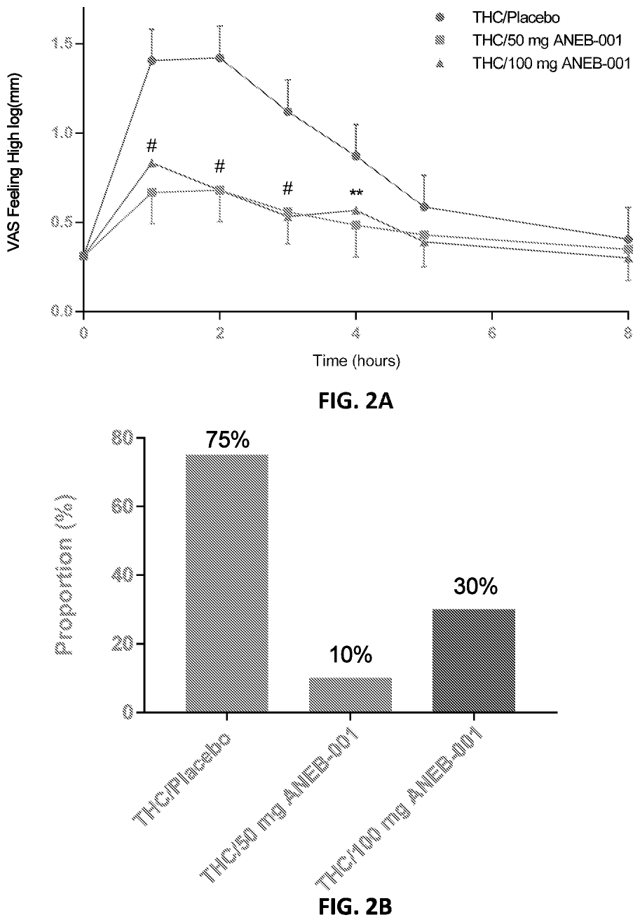

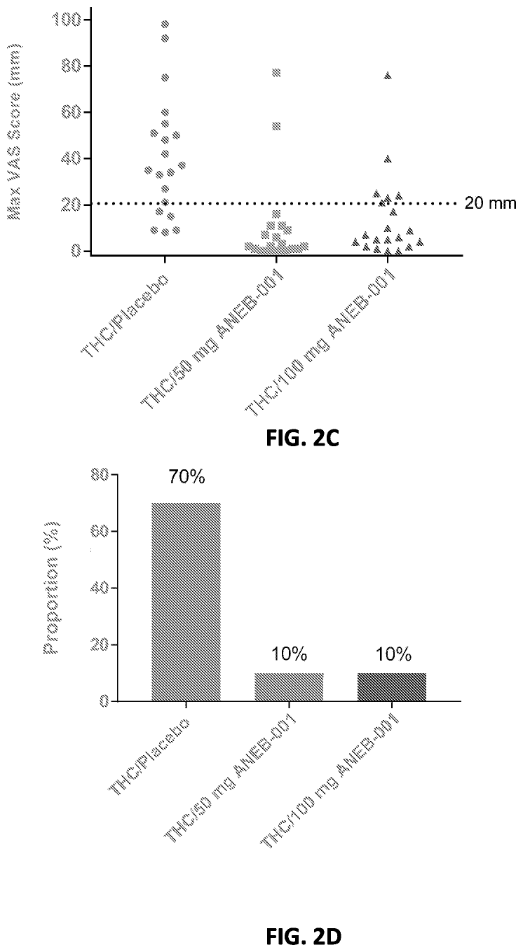

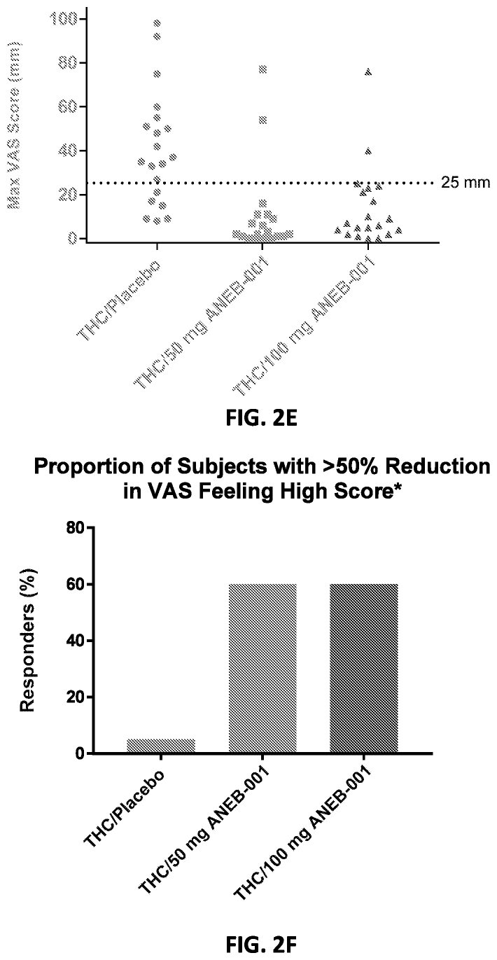

Produced Sustained Reduction of Feeling High (FIG. 2A). Administration of oral THC alone produced a substantial increase in the VAS feeling high score. Coadministration of THC with Compound 1 led to a highly significant reduction in feeling high compared to THC alone (overall p<0.0001) and the effect of Compound 1 in reducing feeling high was sustained for the duration of the THC effect. The 50 mg dose of Compound 1 was as effective as the 100 mg dose.

Reduced the Proportion of Subjects Feeling High (FIG. 2B). 75% of subjects dosed with THC alone reported feeling high, while only 10% of subjects given THC with 50 mg Compound 1 and 30% of subjects given THC with 100 mg Compound 1 reported feeling high. Coadministration of THC with Compound 1 led to a highly significant decrease in the proportion of subjects reporting feeling high (overall p<0.001) and the 50 mg Compound 1 dose was as effective as the 100 mg dose.

Responder Rate for Reduction in Feeling High (FIGS. 2C-2F). 60% of subjects given THC and treated with 50 mg or 100 mg Compound 1 were responders. Coadministration of THC with Compound 1 led to a highly significant increase in the proportion of responders compared to THC alone (p<0.01). The 50 mg dose of Compound 11 was as effective as the 100 mg dose.

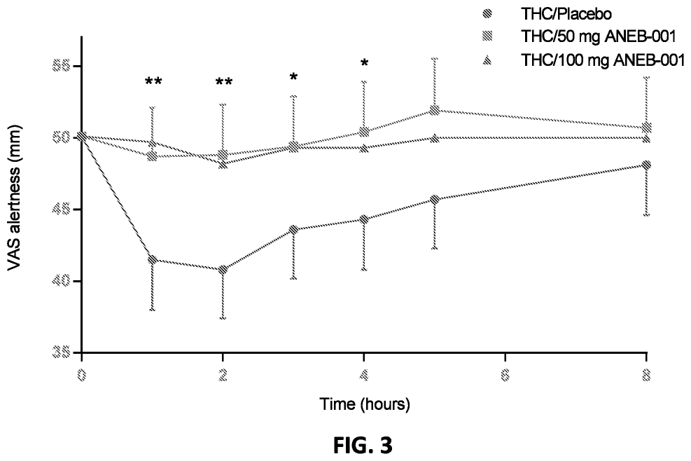

Produced Sustained Improvement in Alertness (FIG. 3). Administration of oral THC alone produced a substantial reduction in alertness. Compound 1 significantly inhibited the reduction in alertness compared to administration of THC alone (overall p<0.01). The effect of Compound 1 on improving alertness was sustained. The 50 mg dose of Compound 1 was as effective as the 100 mg dose.

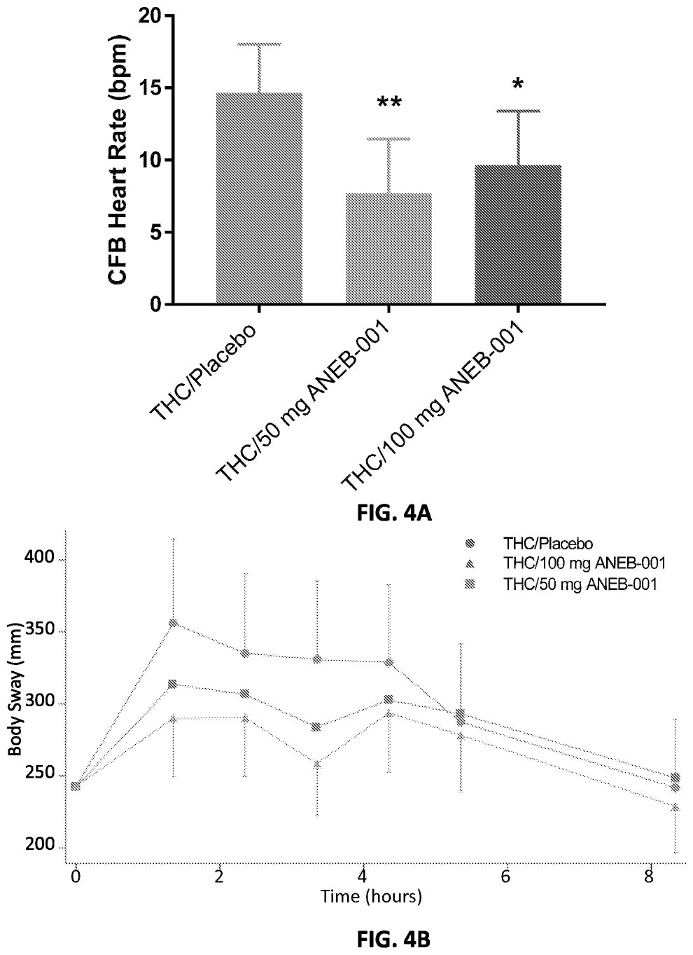

Effect on THC-Induced Body Sway (FIGS. 4A-4B). Administration of oral THC alone produced an increase in body sway, although inter-subject variability was high. Coadministration of THC with Compound 1 showed a trend towards reduced body sway. The effect of Compound 1 on reducing body sway did not reach statistical significance overall. The 50 mg dose of Compound 1 was as effective as the 100 mg dose.

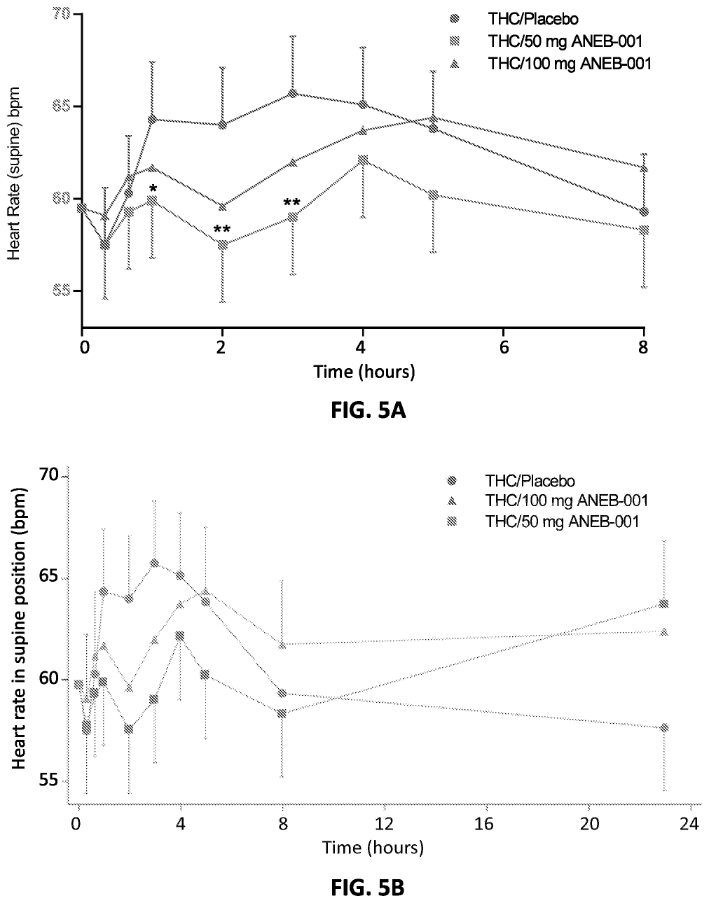

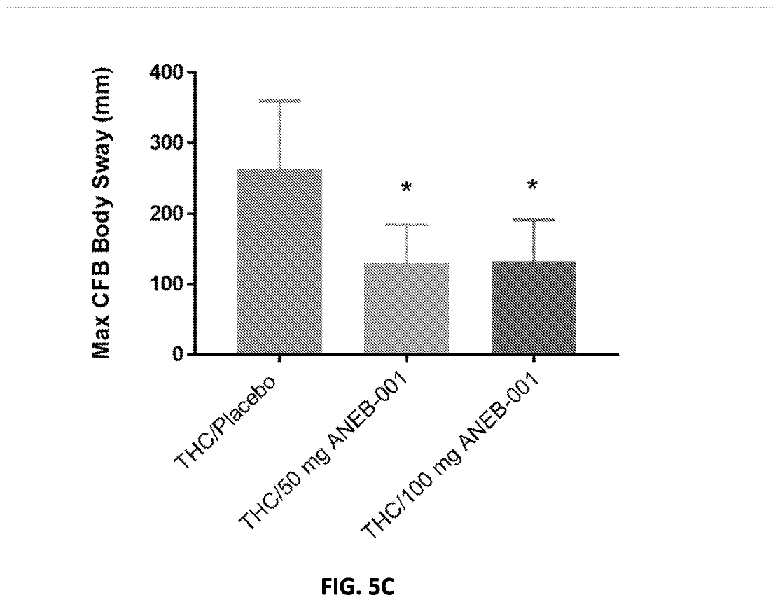

Effect on Heart Rate (FIGS. 5A-5C). Heart rate was measured repeatedly during the study. Administration of THC alone had only a minor effect on heart rate and coadministration of THC with Compound 1 showed a trend towards normalization of heart rate. The effect of Compound 1 on heart rate did not reach statistical significance overall. The 50 mg dose of Compound 1 was as effective as the 100 mg dose. There was no significant overall difference in heart rate for the three treatment arms.

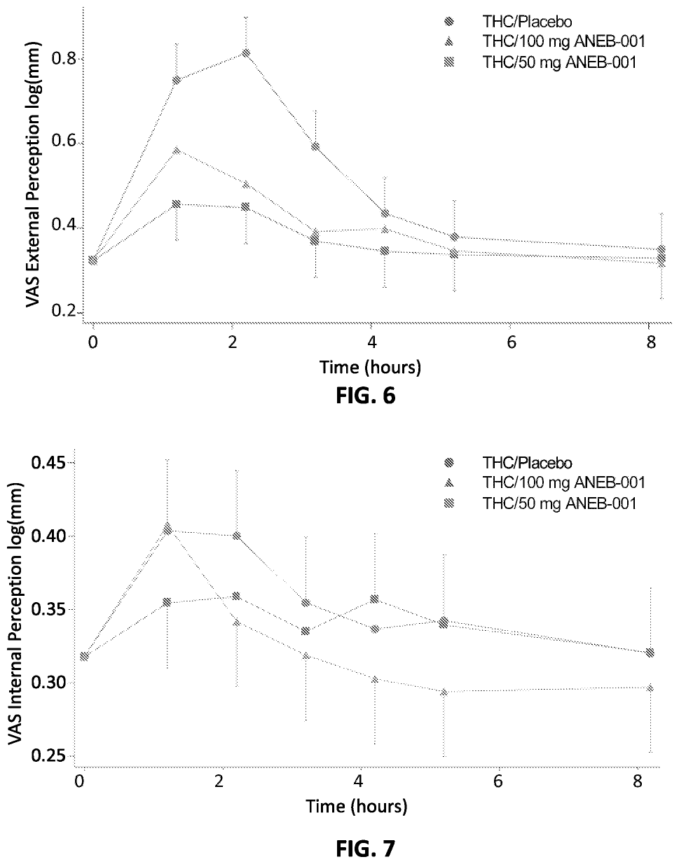

Inhibited THC-Induced Perception Changes (FIG. 6). Administration of oral THC alone had a substantial effect on external perception (misperception of surroundings, body parts, passage of time, colors and sounds, etc.). Coadministration of THC with Compound 1 significantly inhibited the change in perception compared to THC alone (overall p<0.01), and the 50 mg dose of Compound 1 was as effective as the 100 mg dose.

Effect on THC-Induced Perception Changes (FIG. 7). Administration of oral THC alone had a minimal effect on internal perception (misperception of surroundings, body parts, passage of time, colors and sounds, etc.). There were no significant differences between treatments. The 50 mg dose of Compound 1 had a similar effect to the 100 mg dose.

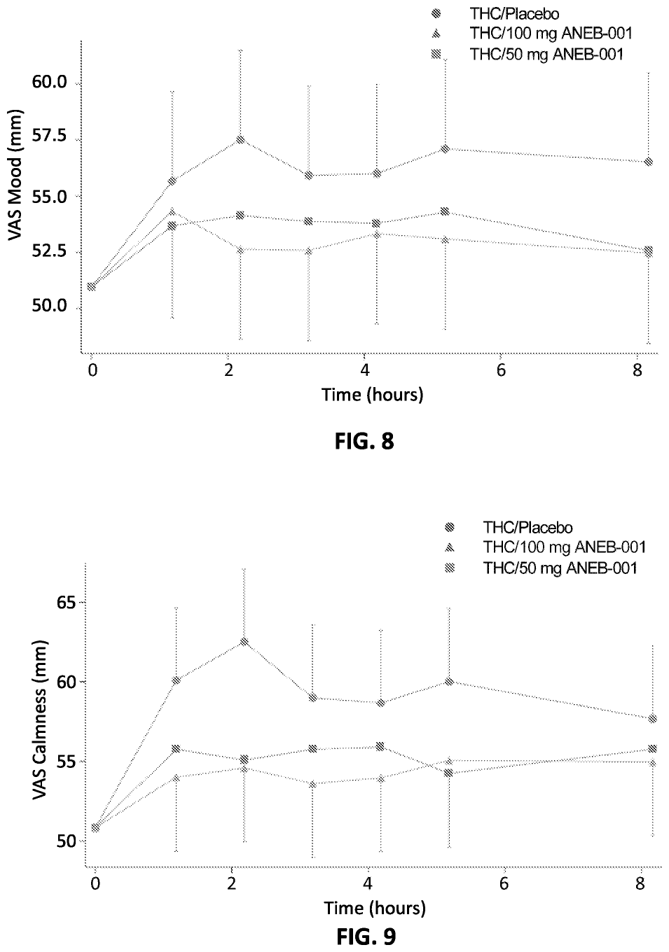

Effect on THC-Induced Mood Changes (FIG. 8). Administration of THC alone had only a minor effect on VAS mood. There were no significant differences between treatments. There was a possible trend to normalization of VAS mood for Compound 1. The 50 mg dose of Compound 11 had a similar effect to the 100 mg dose.

Inhibited THC-Induced Calmness Changes (FIG. 9). Administration of oral THC alone produced a small increase in VAS calmness. There was no overall significant difference between the treatment groups. A possible trend towards normalization of calmness with Compound 1 treatment was observed.

Other secondary outcomes. No significant effects were found for these specific study parameters on saccadic inaccuracy, saccadic peak velocity, saccadic reaction time, smooth pursuit, adaptive tracking, state anxiety score, ave reaction time 0 back, ave reaction time 1 back, ave reaction time 2 back, fraction correct 2 back, left pupil/iris ratio, and right pupil/iris ratio (data not shown).

### Summary

Primary outcomes. VAS Feeling High: Highly significant and sustained improvement Compound 1 (p<0.0001) at both dose levels. Proportion of Subjects Reporting Feeling High on VAS was 75% for THC/placebo versus 10% on 50 mg Compound 1 and 30% on 100 mg Compound 1 (p<0.01). For VAS Alertness, significant improvement in alertness for both 50 mg and 100 mg Compound 1 (p<0.01) was observed. THC effect on body sway was small but trended to improvement for Compound 1, although not statistically significant. THC effect on heart rate was small; Compound 1 showed trend to normalization of heart rate, although not statistically significant. Dose response: 50 mg and 100 mg dose of Compound 1 had similar activity, supports use of a lower dose of Compound 1 and a higher dose of THC.

Secondary outcomes. VAS external perception: Significant improvement at both dose levels (p<0.01). VAS nausea: No significant difference overall between treatment groups. Other secondary VAS scores: No significant differences between treatment groups. Preliminary Safety: All adverse events were mild and transient except in the case of one subject in the 50 mg Compound 1 group who experienced moderate nausea and vomiting.

### Safety Summary

All adverse events were transient and of mild severity, except one case of vomiting of moderate severity in the 50 mg Compound 1 treatment group. One AESI occurred, namely a systolic blood pressure increase >30 mmHg compared to baseline in a subject in the 100 mg Compound 1 group. Nausea, hyperhidrosis and feeling hot were the most frequently observed adverse events in the 50 and 100 mg Compound 1 treatment groups. Nausea was the most common adverse effect (13 subjects in the 50 mg Compound 1 group, 8 subjects in the 100 mg Compound 1 group, 2 subjects in the placebo Compound 1 group). Vomiting occurred in 1 subject in the 50 mg Compound 1 group, 6 subjects in the 100 mg Compound 1 group, and in no subjects in the placebo Compound 1 group. All cases of nausea and vomiting were judged as “probably” or “possibly” related to treatment with Compound 1. These adverse effects are in line with the previously reported dose-dependent emetic effect of Compound 1. Hyperhidrosis occurred in 6 subjects in the 50 mg Compound 1 group, in 7 subjects in the 100 Compound 1 mg group, and in no subjects in the placebo Compound 1 group. Feeling hot occurred in the 6 subjects in the 50 mg Compound 1 group, in 4 subjects in the 100 mg Compound 1 group, an in no subjects in the placebo Compound 1 group. Depressed mood occurred in 1 subject in the 50 mg Compound 1 group, 2 subjects in the 100 mg Compound 1 group, and in no subjects in the placebo Compound 1 group. Adverse effects typical for THC intoxication, like dizziness, euphoric mood and paresthesia mostly occurred in in the Compound 1 placebo group rather than in the Compound 1 50 mg and 100 mg groups.

Vital signs. No consistent and/or clinically relevant abnormalities in blood pressure were observed, except for 1 subject with a systolic blood pressure increase >30 mmHg compared to baseline in the 100 mg Compound 1 group. There was an increase in heart rate (up to ˜6 bpm mean increase) following dosing in the placebo Compound 1 group. The increase was less pronounced in the 50 mg and 100 mg Compound 1 group (up to 3-4 bpm mean increase), consistent with the expected CB1-antagonistic effect of Compound 1. No consistent and/or clinically relevant abnormalities in oxygen saturation or body temperature were observed. Laboratory analyses. An isolated increase was observed in total bilirubin in the 50 and 100 mg Compound 1 groups of 5 mol/L at 24 h post dose compared to baseline, versus 1.4 mol/L increase in placebo group. No consistent and/or clinically relevant abnormalities in other liver enzymes. No consistent and/or clinically relevant abnormalities were observed in other laboratory analyses.

Electrocardiogram (ECG). Slight increases of QTcF were observed in the Compound 1 groups at 3 hours post dose (mean 7.9 ms increase from baseline in 50 mg Compound 1 group, mean 9.9 ms increase from baseline in the 100 mg group, versus mean 3.1 ms increase in placebo group). The QTcF returned to baseline (approximately) at 8 hours post dose for all treatment groups.

Questionnaires. No consistent and/or clinically relevant abnormalities in depressive symptoms (as measured by the BDI) were observed. No suicidality prior or after dosing as assessed by the C-SSRS. Mild dissociative symptoms were observed in some subjects post-dose, as measured by the CADSS; the symptoms normalized at the 24 hour timepoint and during the follow-up visit in all subjects.

Conclusion. After review of the safety data included in this safety report of study CHDR2039, single doses of 50 and 100 mg Compound 1 were safe and well tolerated by most subjects. The observed adverse events were in line with findings from previous research. Minor changes in heart rate, total bilirubin and QTcF are attributable to treatment with Compound 1 and are not considered clinically relevant.

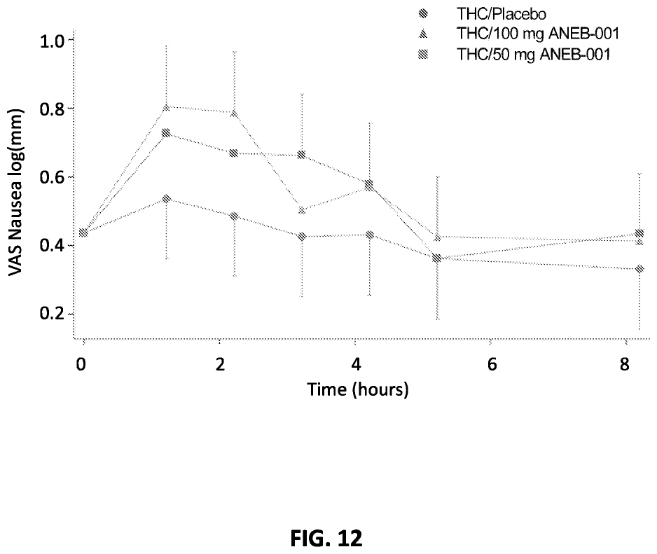

Assessment of nausea by VAS scale was performed repeatedly during the study. There was no overall significant difference in the VAS nausea scores for the three treatment groups (FIG. 12).

### Example 2—PK Evaluation of 50 mg and 100 mg Oral Dosage of Compound 1

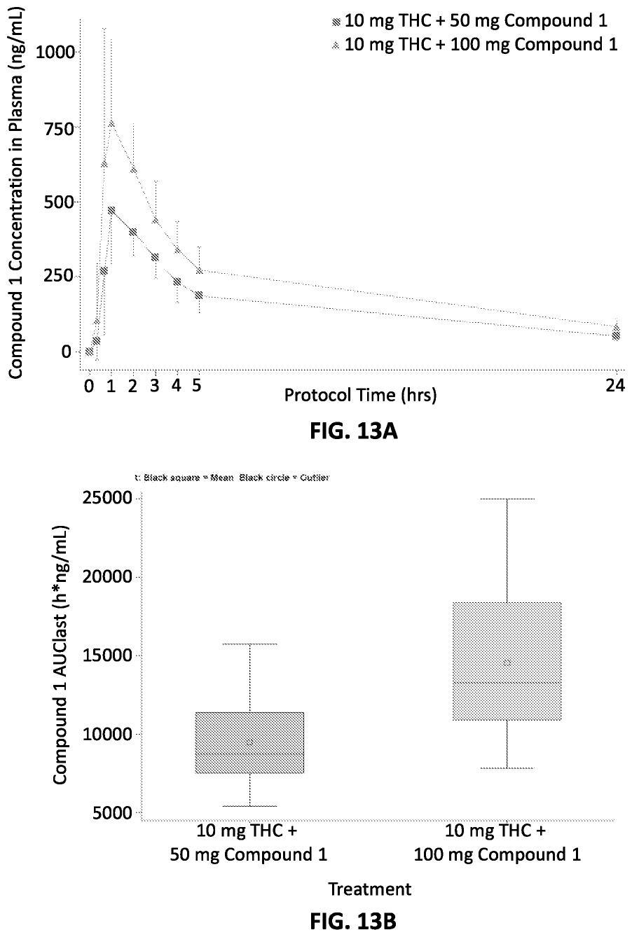

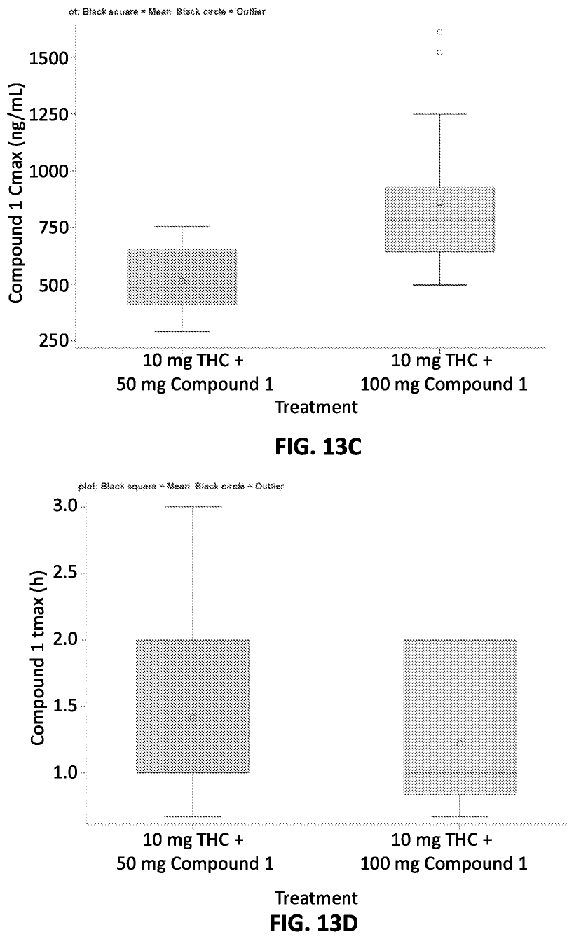

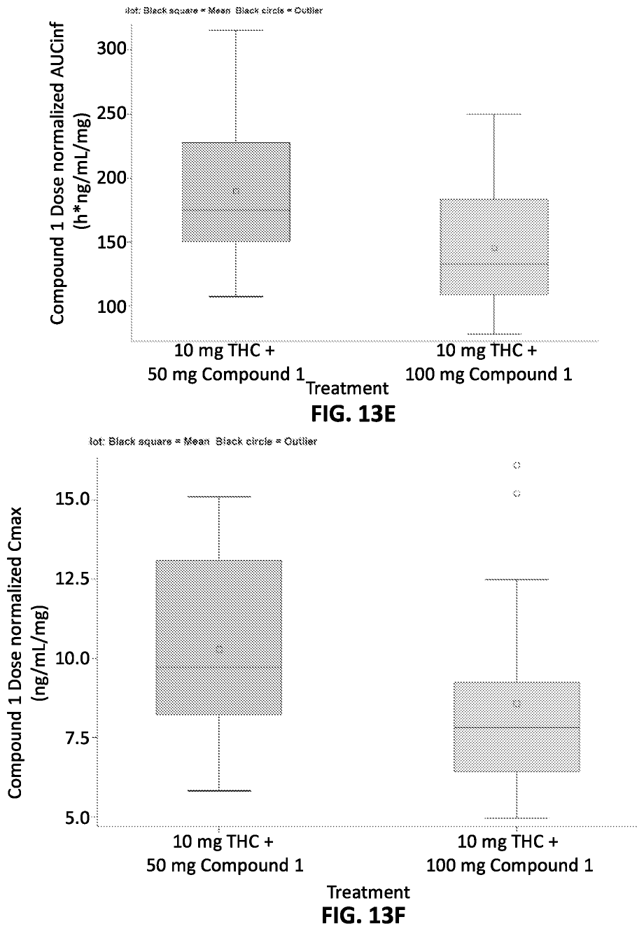

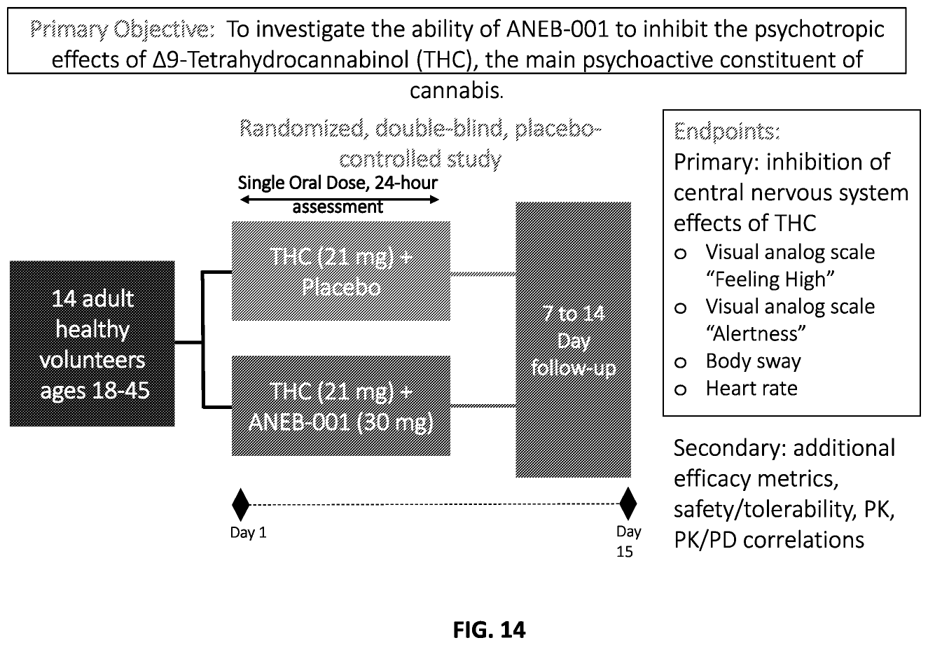

Following the general methods of Example 1, pharmacodynamic properties were measured for Compound 1 (FIGS. 13A-13F). Results are also shown in Tables 1A-2B.

TABLE 1A
Summary of ANEB-001 Concentration in Plasma (ng/mL)
PK Population

| | Protocol | | | | CV | | | |
|---|---|---|---|---|---|---|---|---|
| Treatment | Time (hrs) | N | Mean | SD | (%) | Median | Min | Max |
| 10 mg THC + 50 | 0 | 20 | 0.0 | 0.0 | | 0.0 | 0.0 | 0.0 |
| mg ANEB-001 | 0.33 | 20 | 35.36 | 62.561 | 176.9 | 0.0 | 0.0 | 254 |
| | 0.67 | 20 | 268.0 | 209.59 | 78.2 | 193 | 4.34 | 700 |
| | 1 | 20 | 469.8 | 177.81 | 37.8 | 486 | 117 | 755 |
| | 2 | 20 | 398.9 | 78.554 | 19.7 | 418 | 262 | 566 |
| | 3 | 20 | 312.7 | 67.939 | 21.7 | 310 | 219 | 484 |
| | 4 | 20 | 232.0 | 67.044 | 28.9 | 221 | 133 | 415 |
| | 5 | 20 | 187.0 | 56.554 | 30.3 | 186 | 107 | 343 |
| | 24 | 20 | 52.71 | 14.279 | 27.1 | 52.6 | 28.4 | 84.9 |
| 10 mg THC + 100 | 0 | 20 | 0.0 | 0.0 | | 0.0 | 0.0 | 0.0 |
| mg ANEB-001 | 0.33 | 20 | 105.5 | 188.10 | 178.3 | 37.7 | 0.0 | 822 |
| | 0.67 | 19 | 629.1 | 448.77 | 71.3 | 550 | 22.6 | 1610 |
| | 1 | 20 | 763.7 | 276.59 | 36.2 | 745 | 182 | 1320 |
| | 2 | 20 | 610.8 | 149.04 | 24.4 | 587 | 415 | 917 |
| | 3 | 20 | 441.5 | 128.97 | 29.2 | 409 | 254 | 704 |
| | 4 | 20 | 341.2 | 92.200 | 27.0 | 352 | 172 | 499 |
| | 5 | 20 | 272.8 | 77.040 | 28.2 | 267 | 162 | 414 |
| | 24 | 20 | 84.49 | 26.948 | 31.9 | 83.4 | 47.1 | 164 |

TABLE 1B
ANEB-001 Summary of Plasma PK Parameters
PK Population

| | | | | | Geometric | | CV | Geometric | | | |
|---|---|---|---|---|---|---|---|---|---|---|---|
| Treatment | Parameter | Unit | N | Mean | Mean | SD | (%) | CV (%) | Median | Min | Max |
| 10 mg THC + | AUClast | h*ng/mL | 20 | 9486.3 | 9104.6 | 2856.29 | 30.1 | 29.8 | 8753 | 5375 | 15739 |
| 50 mg | Cmax | ng/mL | 20 | 514.3 | 495.0 | 143.20 | 27.8 | 29.3 | 486 | 291 | 755 |
| ANEB-001 | tlag | h | 20 | | | | | | 0.33 | 0.00 | 0.40 |
| | tmax | h | 20 | | | | | | 1.00 | 0.67 | 3.00 |
| 10 mg THC + | AUClast | h*ng/mL | 20 | 14525.1 | 13684.9 | 5141.00 | 35.4 | 36.7 | 13276 | 7834 | 24989 |
| 100 mg | Cmax | ng/mL | 20 | 857.7 | 809.7 | 316.56 | 36.9 | 35.0 | 782 | 496 | 1610 |
| ANEB-001 | tlag | h | 20 | | | | | | 0.00 | 0.00 | 0.33 |
| | tmax | h | 20 | | | | | | 1.00 | 0.67 | 2.00 |

TABLE 1C
ANEB-001 Summary of Dose-Normalized Plasma PK Parameters
PK Population

| | | | | | Geometric | | CV | Geometric | | | |
|---|---|---|---|---|---|---|---|---|---|---|---|
| Treatment | Parameter | Unit | N | Mean | Mean | SD | (%) | CV (%) | Median | Min | Max |
| 10 mg THC + | Dose | h*ng/mL/mg | 20 | 189.726 | 182.093 | 57.1258 | 30.1 | 29.8 | 175.05 | 107.49 | 314.79 |
| 50 mg | normalized | | | | | | | | | | |
| ANEB-001 | AUClast | | | | | | | | | | |
| | Dose | ng/mL/mg | 20 | 10.285 | 9.900 | 2.8640 | 27.8 | 29.3 | 9.72 | 5.82 | 15.10 |
| | normalized | | | | | | | | | | |
| | Cmax | | | | | | | | | | |
| 10 mg THC + | Dose | h*ng/mL/mg | 20 | 145.251 | 136.849 | 51.4100 | 35.4 | 36.7 | 132.76 | 78.34 | 249.89 |
| 100 mg | normalized | | | | | | | | | | |
| ANEB-001 | AUClast | | | | | | | | | | |
| | Dose | ng/mL/mg | 20 | 8.577 | 8.097 | 3.1656 | 36.9 | 35.0 | 7.82 | 4.96 | 16.10 |
| | normalized | | | | | | | | | | |
| | Cmax | | | | | | | | | | |

TABLE 2A
Summary of THC Concentration in Plasma (ng/mL)
PK Population

| | Protocol | | | | | | | |
|---|---|---|---|---|---|---|---|---|
| Treatment | Time (hrs) | N | Mean | SD | CV (%) | Median | Min | Max |
| 10 mg THC + | 0 | 20 | 0.0 | 0.0 | | 0.0 | 0.0 | 0.0 |
| Placebo | 0.33 | 20 | 1.168 | 1.4843 | 127.1 | 0.776 | 0.0 | 6.74 |
| | 0.67 | 20 | 2.293 | 1.4154 | 61.7 | 1.81 | 0.394 | 5.46 |
| | 1 | 20 | 2.486 | 1.2161 | 48.9 | 2.27 | 0.973 | 5.24 |
| | 2 | 20 | 1.436 | 0.97139 | 67.7 | 1.60 | 0.0 | 4.59 |
| | 3 | 20 | 1.007 | 1.0793 | 107.2 | 0.838 | 0.235 | 5.20 |
| | 4 | 20 | 0.4548 | 0.35150 | 77.3 | 0.443 | 0.108 | 1.73 |
| | 5 | 20 | 0.2850 | 0.20856 | 73.2 | 0.248 | 0.0 | 0.860 |
| | 24 | 20 | 0.0 | 0.0 | | 0.0 | 0.0 | 0.0 |
| 10 mg THC + 50 | 0 | 20 | 0.0 | 0.0 | | 0.0 | 0.0 | 0.0 |
| mg ANEB-001 | 0.33 | 20 | 1.271 | 1.2322 | 96.9 | 0.738 | 0.102 | 4.24 |
| | 0.67 | 20 | 2.902 | 1.6128 | 55.6 | 2.41 | 0.504 | 6.28 |
| | 1 | 20 | 2.756 | 1.0678 | 38.8 | 2.60 | 1.27 | 5.91 |
| | 2 | 20 | 1.398 | 0.78997 | 56.5 | 1.18 | 0.587 | 3.49 |
| | 3 | 20 | 0.7210 | 0.34670 | 48.1 | 0.689 | 0.291 | 1.61 |
| | 4 | 20 | 0.4319 | 0.22078 | 51.1 | 0.379 | 0.178 | 0.953 |
| | 5 | 20 | 0.2875 | 0.13904 | 48.4 | 0.264 | 0.112 | 0.608 |
| | 24 | 20 | 0.005550 | 0.024820 | 447.2 | 0.0 | 0.0 | 0.111 |
| 10 mg THC + 100 | 0 | 20 | 0.0 | 0.0 | | 0.0 | 0.0 | 0.0 |
| mg ANEB-001 | 0.33 | 20 | 1.724 | 1.9489 | 113.0 | 0.986 | 0.152 | 6.55 |
| | 0.67 | 19 | 3.543 | 3.0660 | 86.5 | 2.41 | 0.574 | 12.7 |
| | 1 | 20 | 2.699 | 2.1038 | 78.0 | 1.88 | 1.04 | 10.4 |
| | 2 | 20 | 1.280 | 0.88512 | 69.1 | 1.05 | 0.458 | 4.02 |
| | 3 | 20 | 0.7183 | 0.44109 | 61.4 | 0.639 | 0.226 | 1.97 |
| | 4 | 20 | 0.4460 | 0.29739 | 66.7 | 0.350 | 0.168 | 1.29 |
| | 5 | 20 | 0.3140 | 0.24272 | 77.3 | 0.228 | 0.123 | 0.938 |
| | 24 | 20 | 0.006000 | 0.026833 | 447.2 | 0.0 | 0.0 | 0.120 |

TABLE 2B
THC Summary of Plasma PK Parameters
PK Population

| | | | | | Geometric | | CV | Geometric | | | |
|---|---|---|---|---|---|---|---|---|---|---|---|
| Treatment | Parameter | Unit | N | Mean | Mean | SD | (%) | CV (%) | Median | Min | Max |
| 10 mg THC + | AUClast | h*ng/mL | 20 | 5.722 | 5.161 | 2.7366 | 47.8 | 49.4 | 4.96 | 2.33 | 13.27 |
| Placebo | Cmax | ng/mL | 20 | 3.147 | 2.758 | 1.5857 | 50.4 | 58.8 | 2.81 | 0.97 | 6.74 |
| | tlag | h | 20 | | | | | | 0.00 | 0.00 | 0.33 |
| | tmax | h | 20 | | | | | | 1.00 | 0.33 | 3.02 |
| 10 mg + THC | AUClast | h*ng/mL | 20 | 5.976 | 5.651 | 2.2007 | 36.8 | 34.4 | 5.51 | 3.37 | 12.52 |
| 50 mg | Cmax | ng/mL | 20 | 3.395 | 3.144 | 1.3539 | 39.9 | 42.7 | 3.12 | 1.27 | 6.28 |
| ANEB-001 | tlag | h | 20 | | | | | | 0.00 | 0.00 | 0.00 |
| | tmax | h | 20 | | | | | | 1.00 | 0.67 | 2.02 |
| 10 mg + THC | AUClast | h*ng/mL | 20 | 6.128 | 5.297 | 4.0333 | 65.8 | 56.0 | 4.94 | 2.75 | 20.41 |
| 100 mg | Cmax | ng/mL | 20 | 3.769 | 3.078 | 2.8414 | 75.4 | 68.3 | 2.46 | 1.19 | 12.70 |
| ANEB-001 | tlag | h | 20 | | | | | | 0.00 | 0.00 | 0.00 |
| | tmax | h | 20 | | | | | | 0.67 | 0.33 | 2.00 |

### Example 3—PD Evaluation of 30 mg Oral Dosage of Compound 1

Following the general methods of Example 1, the study design was adjusted such that groups received either (a) THC (10.5 mg)+placebo or (b) THC (10.5 mg)+Compound 1 (30 mg). Pharmacodynamic properties were then measured for Compound 1. Treatment with Compound 1 resulted in improvements in metrics such as body sway, VAS alertness, and VAS feeling high vs. placebo.

### Example 4—PK and PD Evaluation of 30 mg Oral Dosage of Compound 1 and 10 mg Oral Dosage of Compound 1

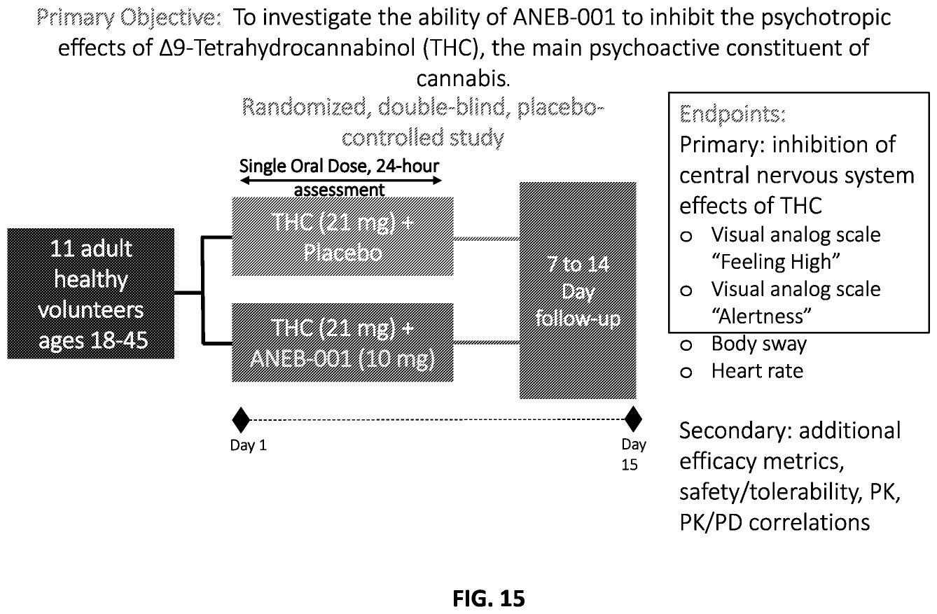

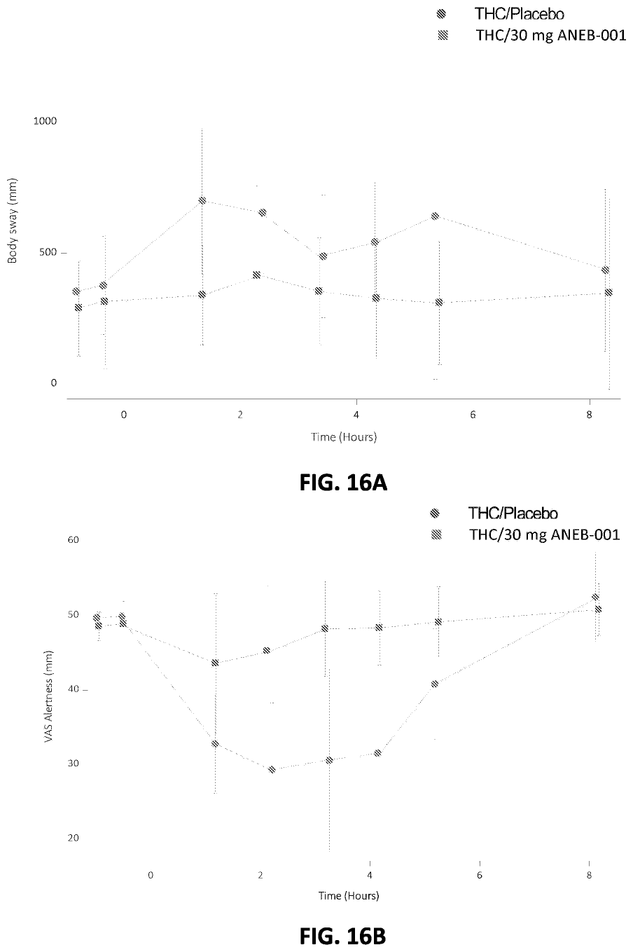

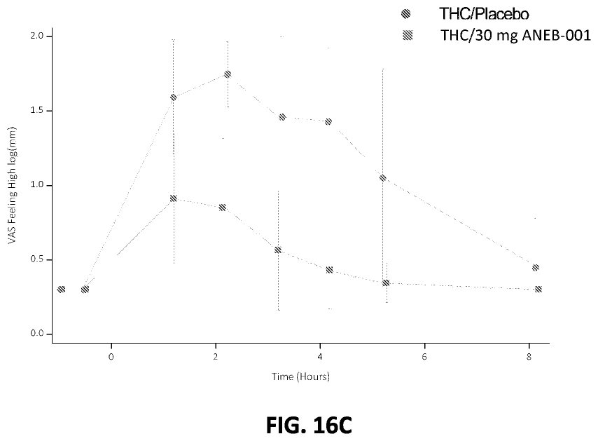

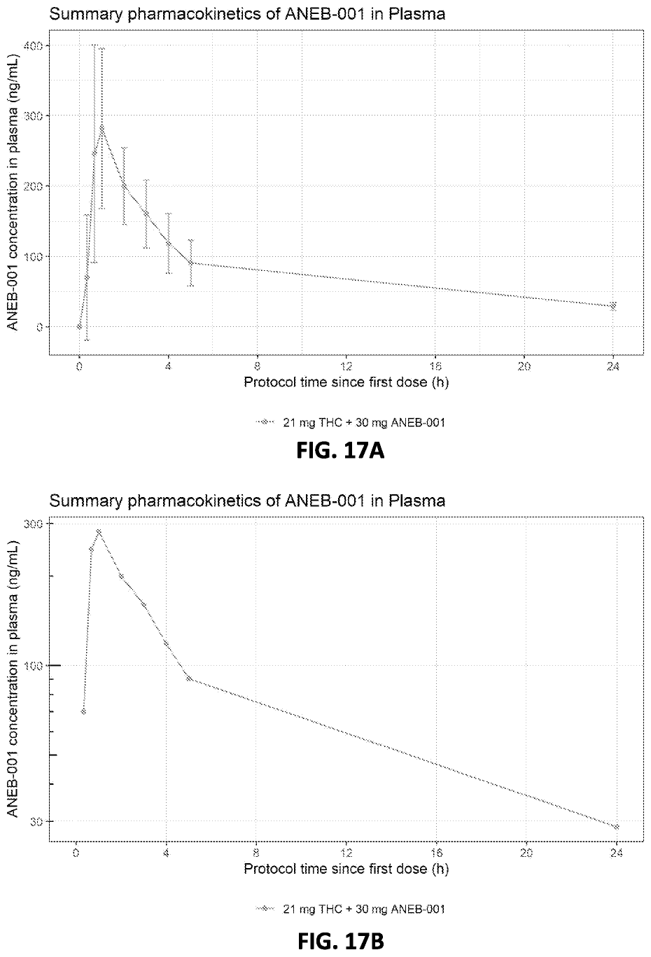

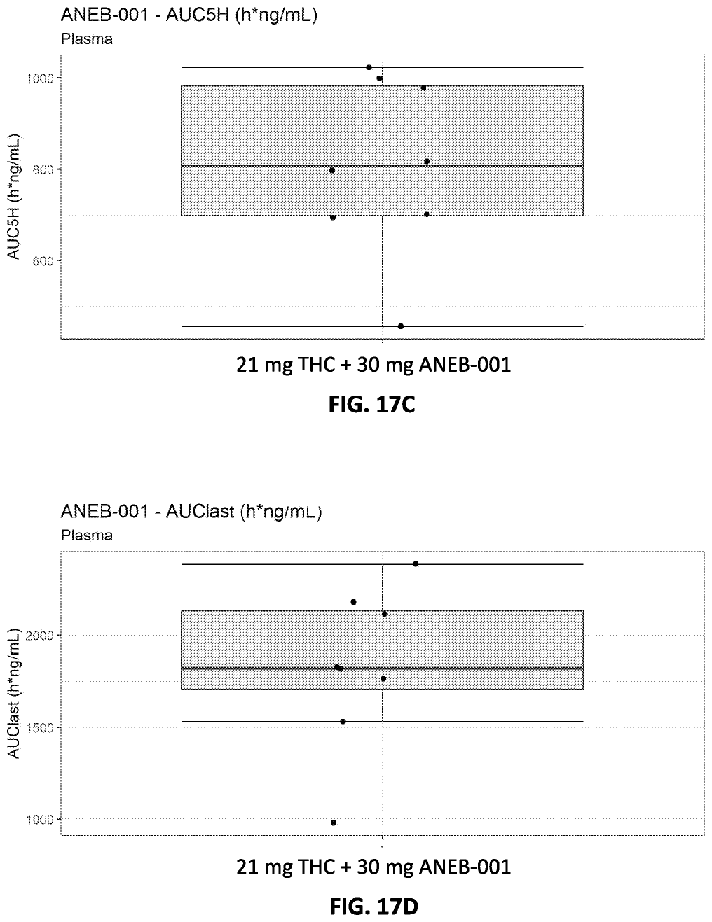

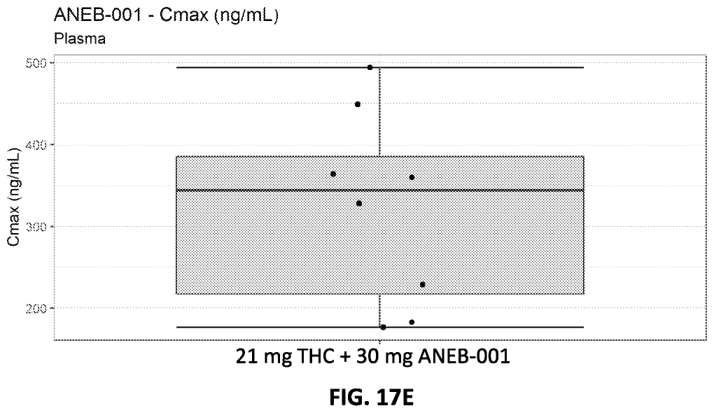

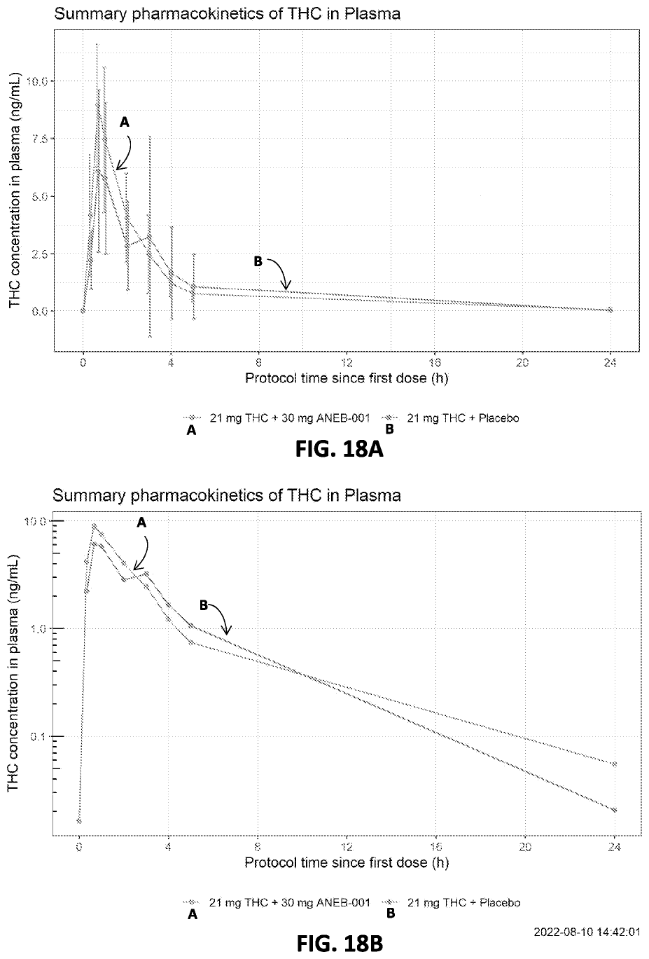

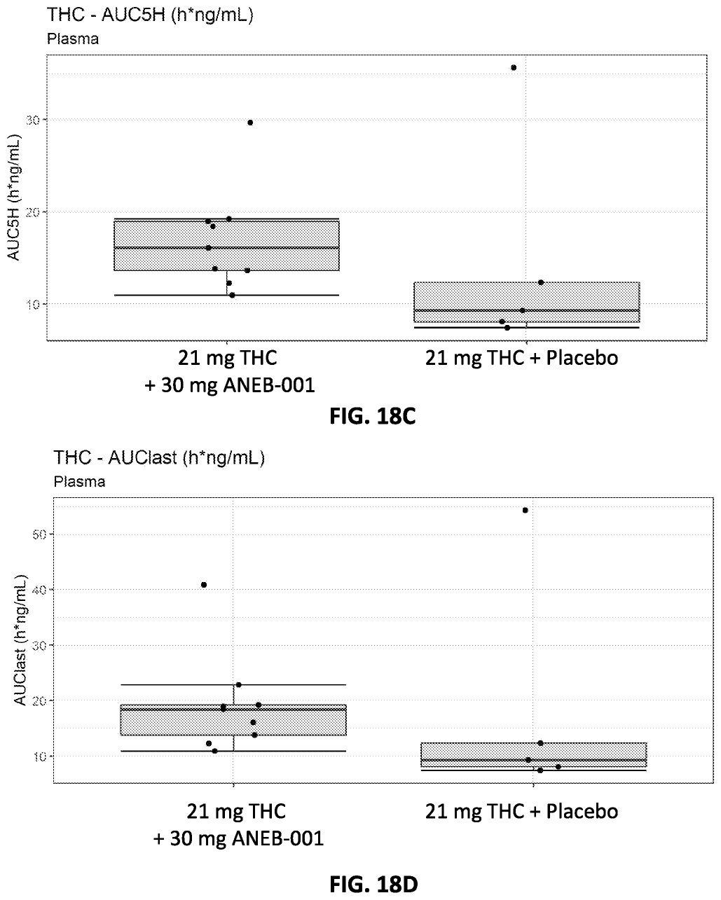

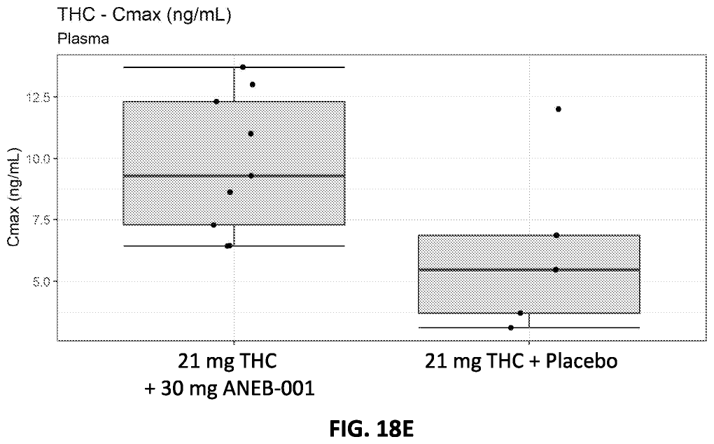

Following the general methods of Example 2, the study design was adjusted such that groups received either (a) THC (21 mg)+placebo or (b) THC (21 mg)+Compound 1 (30 mg) (FIG. 14) or either (a) THC (21 mg)+placebo or (b) THC (21 mg)+Compound 1 (10 mg) (FIG. 15). FIGS. 16A-16C depicts pharmacodynamic properties measured for challenge with 30 mg Compound 1 (ANEB-001) or 10 mg of Compound 1 (ANEB-001) vs. placebo, with improvements in Body Sway (FIG. 16A), Alertness (FIG. 16B) and Feeling High (FIG. 16C) for 30 mg ANEB-001. Pharmacokinetic measurements are shown in FIGS. 17A-18E: Summary of pharmacokinetics for 30 mg ANEB-001 in plasma (FIGS. 17A and 17B); AUC after 5 hours for 30 mg ANEB-001 (FIG. 17C), AUC last (FIG. 17D), and Cmax (FIG. 17E). Summary of pharmacokinetics of THC with 30 mg ANEB-001 challenge (FIG. 18A and FIG. 18B), AUC after 5 hours (FIG. 18C), AUC last (FIG. 18D) and Cmax (FIG. 18E).

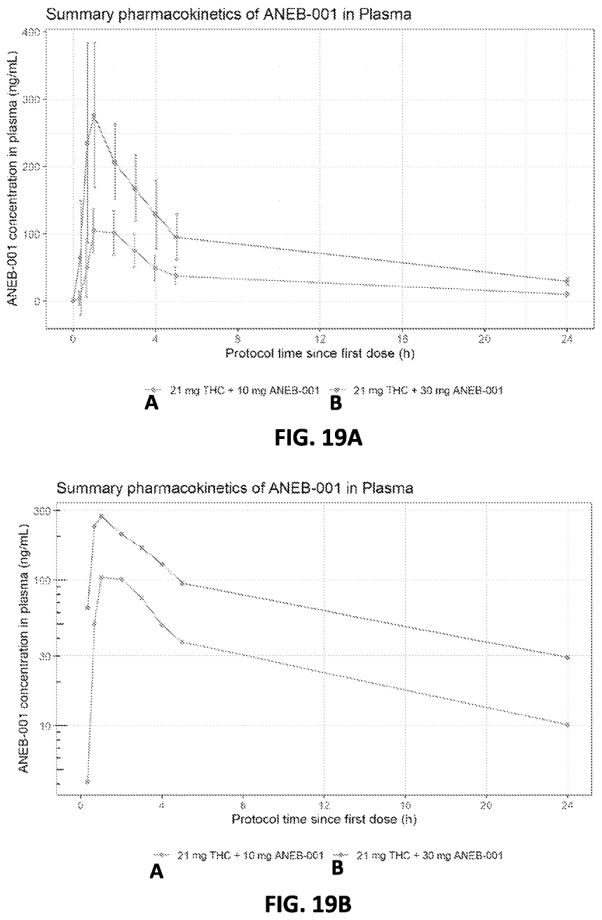

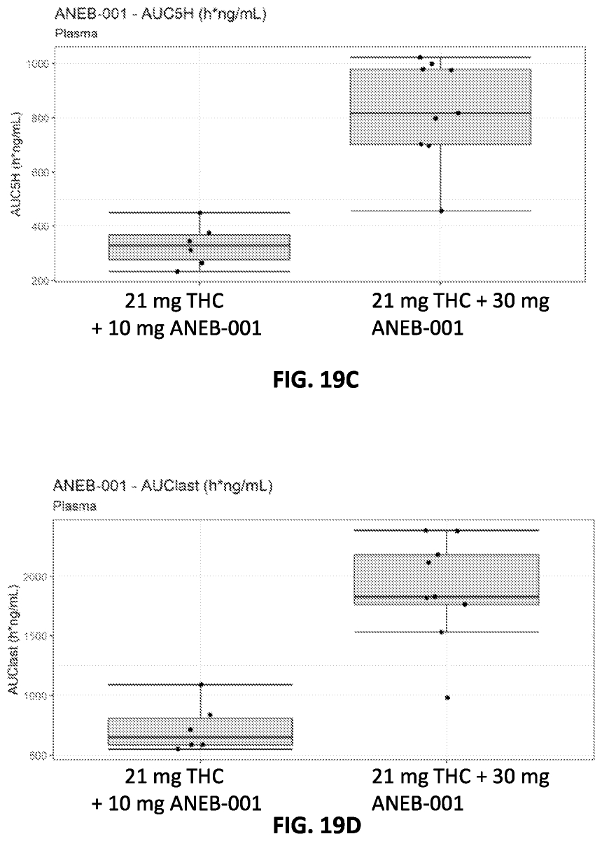

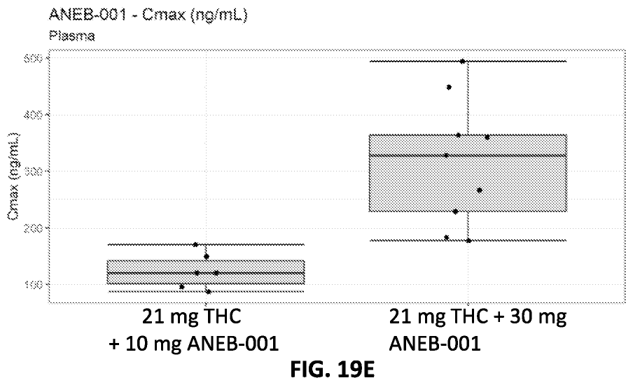

Summary of pharmacokinetics for 30 mg ANEB-001 and 10 mg ANEB-001 challenge with 21 mg THC administration in plasma (FIGS. 19A and 19B); AUC after 5 hours for 10 mg ANEB-001 and 30 mg ANEB-001 (FIG. 19C), AUC last (FIG. 19D), and Cmax (FIG. 19E).

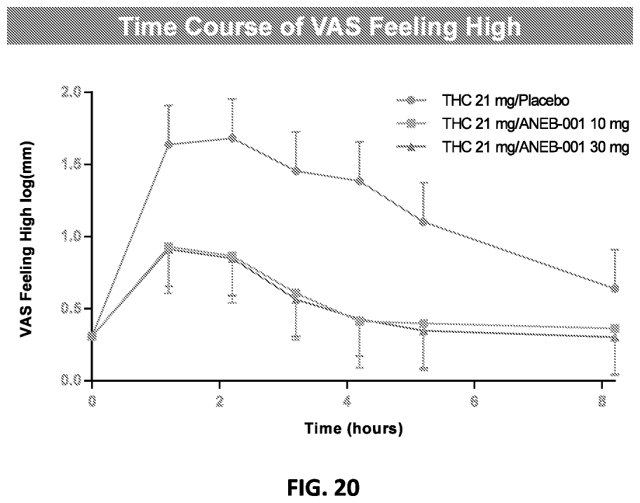

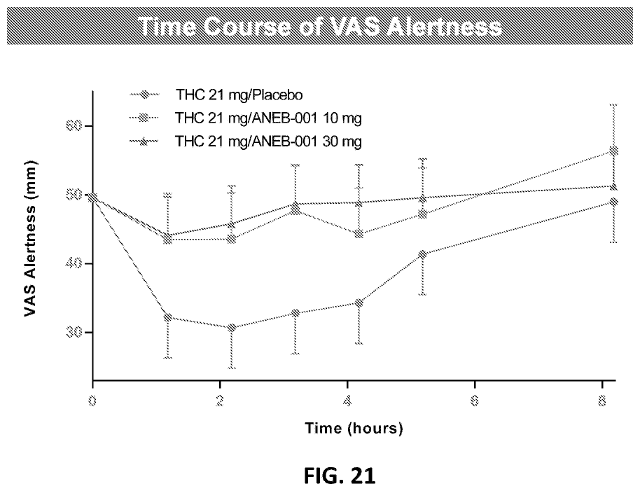

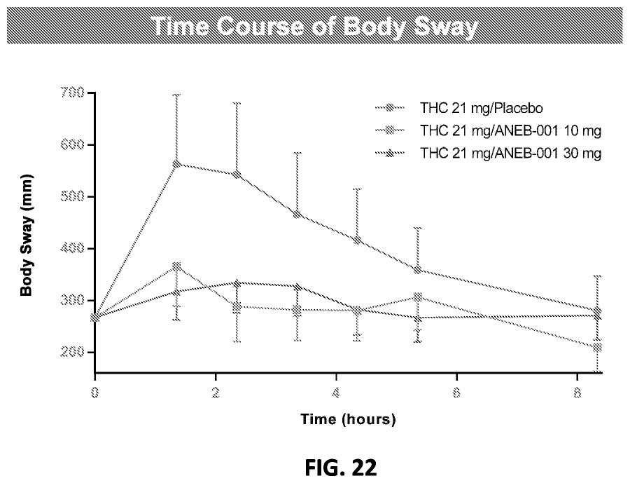

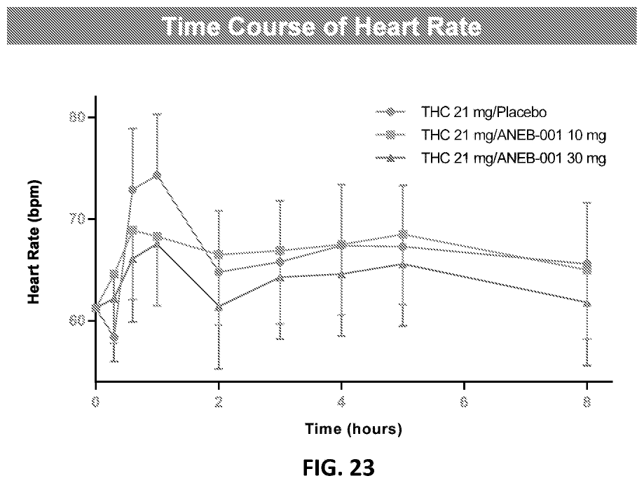

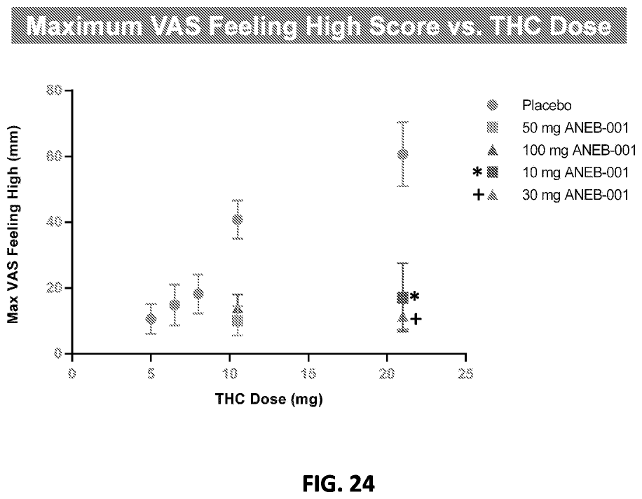

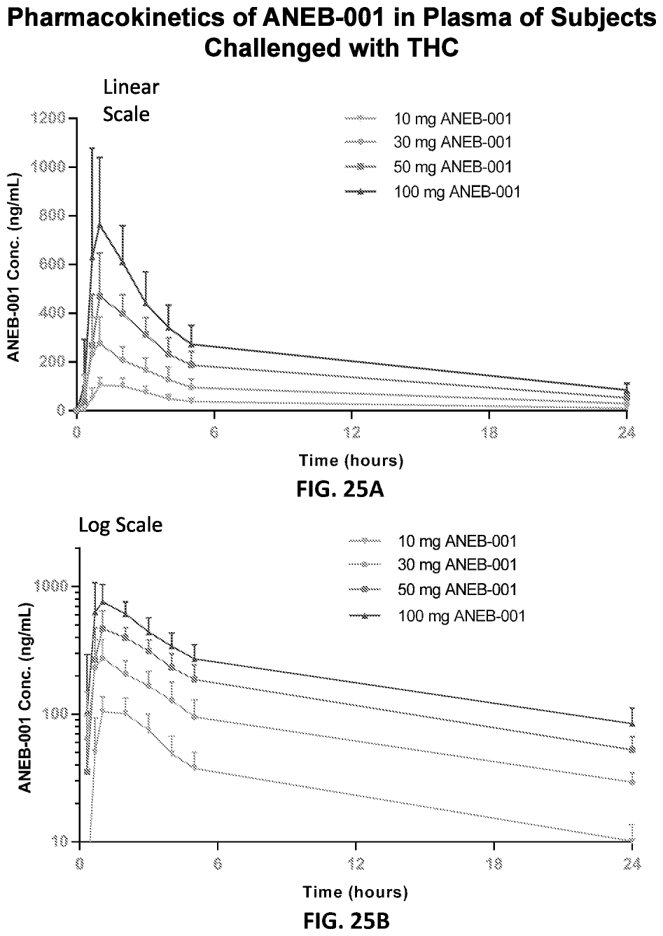

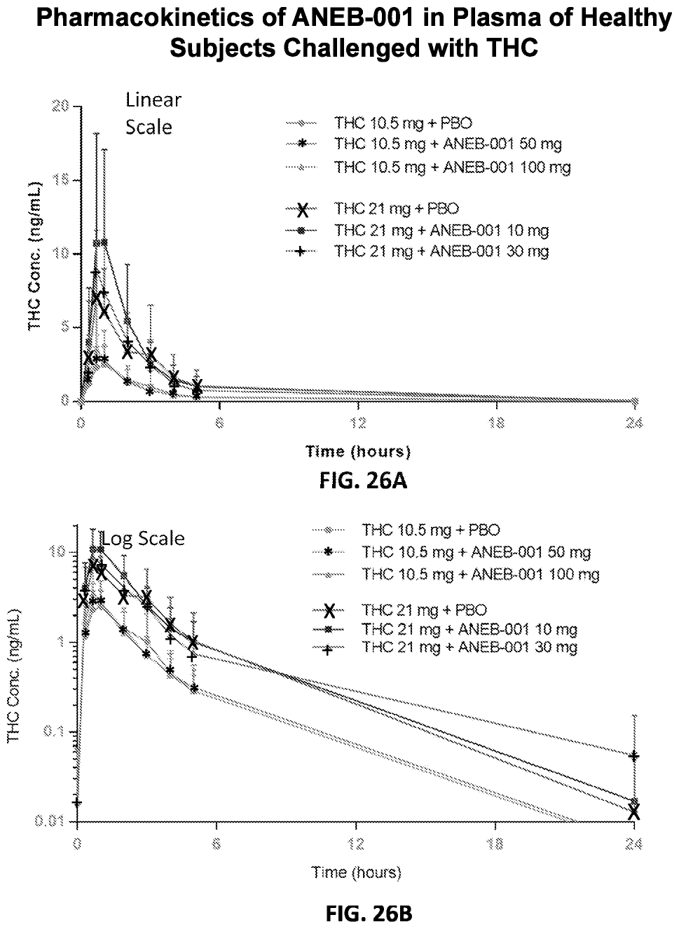

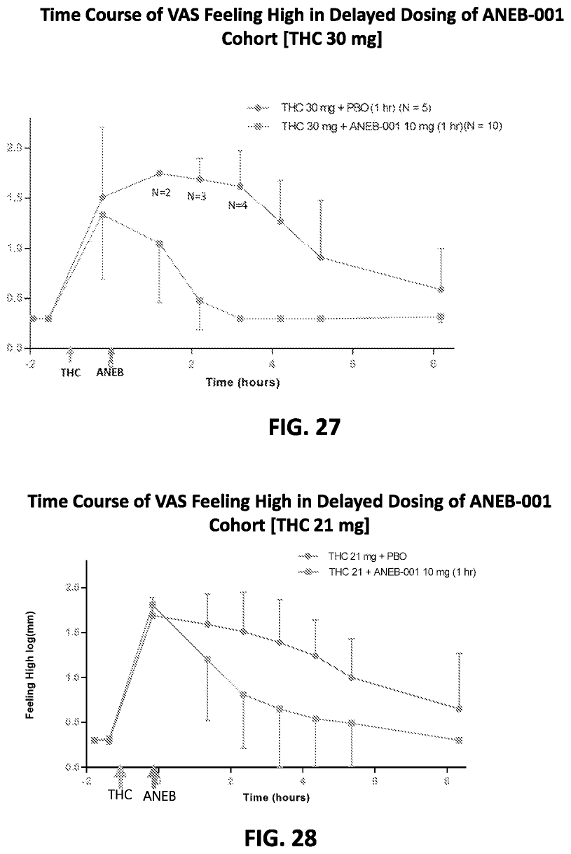

FIGS. 20-24 provide summaries of pharmacodynamic measurements after 10 mg ANEB-001, 30 mg ANEB-001, 50 mg ANEB-001 and 100 mg ANEB-001 challenges vs. placebo: Feeling High (FIG. 20), Alertness (FIG. 21), Body Sway (FIG. 22), and Heart Rate (FIG. 23). FIG. 24 shows Feeling High VAS scores vs. THC dose. FIGS. 25-26 provide summaries of pharmacokinetic measurements over time with 10 mg ANEB-001, 30 mg ANEB-001, 50 mg ANEB-001 and 100 mg ANEB-001 challenge vs. placebo: FIGS. 25A (linear scale) and 25B (log scale) provide ANEB-001 concentrations (ng/mL) over 0-24 hours. FIGS. 26A (linear scale) and 26B (log scale) provide THC concentrations over 0-24 hours.

Results are also shown in Tables 3A-5B.

All PK parameter calculations were based on actual sampling time. For calculation of PK parameters, all BLQ concentrations that were between the first and last non-BLQ values were dropped (treated as ‘missing’). For calculation of AUClast and t1/2, additionally all values that were BLQ after the last non-BLQ value were dropped (treated as missing). If data points for concentrations were missing, the AUC parameters were derived by interpolating with regard to the 2 neighboring non-missing concentrations. AUC was calculated using the log-linear trapezoidal rule. If the same maximum concentrations were observed at 2 or more time points, the earliest time point will be used as tmax.

Reporting of drug concentration data. The concentrations are summarized (mean, SD, % CV, median, Min and Max values) by treatment and time, and are also presented graphically as mean over time, with standard deviation as error bars. For graphical and summary purposes of drug concentrations, values below the limit of quantification (BLQ) are set to zero. The SD and CVT are not reported if the mean is 0.

Reporting of PK parameters. The individual PK parameters (except tmax and tlag) are summarized (mean, SD, CV, geometric mean, geometric CV, median, M in and Max) per treatment group and a selection of the PK parameters are presented graphically as boxplots. For tmax and tlag, the median, Min and Max statistics are reported. Boxplots present the median (horizontal solid line), the 25%-75% distribution (IQR), with 1.5× the IQR as whiskers.

TABLE 3A
Summary of PK of ANEB-001 in Plasma

| | Protocol | | | | | | |
|---|---|---|---|---|---|---|---|
| Treatment | Time | Mean | SD | Median | CV % | Min | Max |
| 21 mg THC + 30 | 00H00M | 0 | | 0 | | 0 | 0 |
| mg ANEB-001 | 00H20M | 69.89 | 88.934 | 19.3 | 127.2 | 0 | 232 |
| | 00H40M | 246.1 | 154.85 | 278 | 62.9 | 54.0 | 494 |
| | 01H00M | 281.9 | 113.89 | 317 | 40.4 | 130 | 449 |
| | 02H00M | 199.8 | 54.670 | 180 | 27.4 | 131 | 277 |
| | 03H00M | 160.2 | 48.209 | 168 | 30.1 | 85.2 | 229 |
| | 04H00M | 118.2 | 42.496 | 122 | 36 | 56.0 | 180 |
| | 05H00M | 90.44 | 32.842 | 95.3 | 36.3 | 40.2 | 146 |
| | 24H00M | 28.74 | 5.5045 | 28.5 | 19.2 | 18.0 | 36.9 |

TABLE 3B
Summary of PK parameters of ANEB-001 in Plasma

| | | | | | CV | | GeoCV | | | |
|---|---|---|---|---|---|---|---|---|---|---|
| Treatment | Parameter | unit | Mean | SD | % | GeoMean | % | Median | Min | Max |
| 21 mg THC + | AUC5H | h*ng/mL | 808 | 193 | 23.9 | 785 | 27.3 | 807 | 456 | 1023 |
| 30 mg | AUClast | h*ng/mL | 1825 | 436 | 23.9 | 1771 | 28.2 | 1822 | 981 | 2386 |
| ANEB-001 | Cmax | ng/mL | 323.0 | 118.29 | 36.6 | 302.8 | 40.9 | 344 | 177 | 494 |
| | Dose | ng/mL/mg | 10.77 | 3.9429 | 36.6 | 10.09 | 40.9 | 11.5 | 5.90 | 16.5 |
| | normalized | | | | | | | | | |
| | Cmax | | | | | | | | | |
| | tlag | h | | | | | | 0 | 0 | 0.33 |
| | tmax | h | | | | | | 1 | 0.67 | 3 |

TABLE 4A
Summary of PK of THC in Plasma

| | Protocol | | | | | | |
|---|---|---|---|---|---|---|---|
| Treatment | Time | Mean | SD | Median | CV % | Min | Max |
| 21 mg THC + | 00H00M | 0 | | 0 | | 0 | 0 |
| Placebo | 00H20M | 2.222 | 1.2738 | 2.01 | 57.3 | 0.640 | 4.19 |
| | 00H40M | 6.100 | 3.5333 | 5.46 | 57.9 | 3.11 | 12.0 |
| | 01H00M | 5.776 | 3.2889 | 5.47 | 56.9 | 2.82 | 10.8 |
| | 02H00M | 2.850 | 1.9410 | 1.86 | 68.1 | 1.73 | 6.27 |
| | 03H00M | 3.228 | 4.3502 | 1.47 | 134.8 | 0.949 | 11.0 |
| | 04H00M | 1.658 | 2.0039 | 0.894 | 120.9 | 0.544 | 5.22 |
| | 05H00M | 1.063 | 1.4130 | 0.456 | 133 | 0.395 | 3.59 |
| | 24H00M | 0.02060 | 0.046063 | 0 | 223.6 | 0 | 0.103 |
| 21 mg THC + 30 | 00H00M | 0.01633 | 0.049000 | 0 | 300 | 0 | 0.147 |
| mg ANEB-001 | 00H20M | 4.161 | 2.6468 | 3.48 | 63.6 | 1.43 | 9.28 |
| | 00H40M | 8.943 | 2.6666 | 8.40 | 29.8 | 6.42 | 13.7 |
| | 01H00M | 7.449 | 3.1616 | 7.31 | 42.4 | 3.28 | 12.3 |
| | 02H00M | 4.069 | 1.9322 | 4.01 | 47.5 | 1.82 | 8.13 |
| | 03H00M | 2.465 | 1.7242 | 2.03 | 69.9 | 0.800 | 6.52 |
| | 04H00M | 1.216 | 0.61433 | 1.28 | 50.5 | 0.480 | 2.36 |
| | 05H00M | 0.7443 | 0.35064 | 0.684 | 47.1 | 0.314 | 1.20 |
| | 24H00M | 0.05514 | 0.096965 | 0 | 175.8 | 0 | 0.233 |

TABLE 4B
Summary of PK parameters of THC in Plasma

| | | | | | CV | | GeoCV | | | |
|---|---|---|---|---|---|---|---|---|---|---|
| Treatment | Parameter | unit | Mean | SD | % | GeoMean | % | Median | Min | Max |
| 21 mg THC + | AUC5H | h*ng/mL | 14.6 | 11.9 | 81.9 | 12 | 71.1 | 9.32 | 7.43 | 35.6 |
| Placebo | AUClast | h*ng/mL | 18.3 | 20.2 | 110.5 | 13 | 98.1 | 9.32 | 7.43 | 54.3 |
| | Cmax | ng/mL | 6.226 | 3.5493 | 57 | 5.530 | 57.4 | 5.47 | 3.11 | 12.0 |
| | Dose | ng/mL/mg | 0.2965 | 0.16901 | 57 | 0.2634 | 57.4 | 0.260 | 0.148 | 0.571 |
| | normalized | | | | | | | | | |
| | Cmax | | | | | | | | | |
| | tlag | h | | | | | | 0 | 0 | 0 |
| | tmax | h | | | | | | 0.67 | 0.67 | 1 |
| 21 mg THC + | AUC5H | h*ng/mL | 17 | 5.62 | 33.1 | 16.3 | 30.7 | 16.1 | 10.9 | 29.7 |
| 30 mg | AUClast | h*ng/mL | 19.3 | 8.94 | 46.4 | 17.9 | 40.5 | 18.4 | 10.9 | 40.9 |
| ANEB-001 | Cmax | ng/mL | 9.781 | 2.8259 | 28.9 | 9.412 | 30.4 | 9.28 | 6.42 | 13.7 |
| | Dose | ng/mL/mg | 0.4658 | 0.13457 | 28.9 | 0.4482 | 30.4 | 0.442 | 0.306 | 0.652 |
| | normalized | | | | | | | | | |
| | Cmax | | | | | | | | | |
| | tlag | h | | | | | | 0 | 0 | 0 |
| | tmax | h | | | | | | 0.67 | 0.33 | 1 |

TABLE 5A
Summary of PK parameters of ANEB-001 in Plasma

| | | | | | CV | | GeoCV | | | |
|---|---|---|---|---|---|---|---|---|---|---|
| Treatment | Parameter | unit | Mean | SD | % | GeoMean | % | Median | Min | Max |
| 21 mg THC + | AUC5H | h*ng/mL | 330 | 78.3 | 23.7 | 322 | 24.2 | 329 | 233 | 449 |
| 10 mg | AUClast | h*ng/mL | 729 | 207 | 28.5 | 707 | 26.8 | 652 | 552 | 1093 |
| ANEB-001 | Cmax | ng/mL | 123.6 | 31.428 | 25.4 | 120.3 | 25.8 | 120 | 87.2 | 170 |
| | Dose | ng/mL/mg | 12.36 | 3.1428 | 25.4 | 12.03 | 25.8 | 12.0 | 8.72 | 17.0 |
| | normalized | | | | | | | | | |
| | Cmax | | | | | | | | | |
| | tlag | h | | | | | | 0 | 0 | 0.33 |
| | tmax | h | | | | | | 1.5 | 1 | 2 |

TABLE 5B
Summary of PK of ANEB-001 in Plasma

| | Protocol | | | | | | |
|---|---|---|---|---|---|---|---|
| Treatment | Time | Mean | SD | Median | CV % | Min | Max |
| 21 mg THC + | 00H00M | 0 | | 0 | | 0 | 0 |
| 10 mg | 00H20M | 4.133 | 10.125 | 0 | 244.9 | 0 | 24.8 |
| ANEB-001 | 00H40M | 50.00 | 43.697 | 33.6 | 87.4 | 27.1 | 139 |
| | 01H00M | 104.8 | 32.781 | 95.2 | 31.3 | 80.0 | 170 |
| | 02H00M | 101.2 | 33.243 | 100 | 32.8 | 68.4 | 149 |
| | 03H00M | 75.13 | 25.163 | 69.6 | 33.5 | 46.5 | 112 |
| | 04H00M | 49.23 | 18.205 | 40 | 37 | 33.4 | 79.4 |
| | 05H00M | 37.63 | 12.838 | 33.7 | 34.1 | 26.6 | 60.1 |
| | 24H00M | 10.16 | 3.4623 | 9.29 | 34.1 | 6.85 | 16.5 |

### Example 5—PD and PK Evaluation of 1-30 mg Oral Dosage of Compound 1

Following the general methods of Example 1, the study design was adjusted such that groups received either (a) THC (10.5 mg)+placebo; (b) THC (10.5 mg)+Compound 1 (20 mg); (c) THC (10.5 mg)+Compound 1 (15 mg); (d) THC (10.5 mg)+Compound 1 (10 mg); (e) THC (10.5 mg)+Compound 1 (5 mg); or (f) THC (10.5 mg)+Compound 1 (1 mg). Endpoints were then evaluated as generally described in Example 1, and PK properties were measured as shown for the general methods of Example 2.

### Example 6—Delayed Compound 1 Administration

Following the general methods of Examples 1-3, the study design was adjusted such that placebo or Compound 1 were administered 30 min, 1 hour, 2 hours, 3 hours, 5 hours, 8 hours, or 12 hours after administration of THC. Endpoints were then evaluated as generally described in Example 1, and PK properties were measured as shown for the general methods of Example 2.

### Example 7—Increased THC Challenge Dose

Following the general methods of Examples 1-4, the study design was adjusted such that groups received a challenge dose of 21 mg, 50 mg, 100 mg, 200 mg, or 500 mg of THC. Endpoints were then evaluated as generally described in Example 1, and PK properties were measured as shown for the general methods of Example 2.

### Example 8—PD and PK Evaluation of 10-30 mg Oral Dosage of Compound 1

Following the general methods of Example 1, the study design was adjusted such that groups received either (a) THC (21 mg)+Compound 1 (30 mg); and (b) THC (21 mg)+Compound 1 (10 mg). Endpoints were then evaluated as generally described in Example 1, and PK properties were measured as shown for the general methods of Example 2.

### Example 9—Delayed Compound 1 Administration and Increased THC Challenge

Following the general methods of Examples 1-3, the study design was adjusted such that Compound 1 was administered 1 hour after administration of THC. Groups received either (a) THC (21 mg)+Compound 1 (10 mg); (b) THC (40 mg)+Compound 1 (10 mg); (c) THC (30 mg)+Compound 1 (10 mg); or (d) THC (30 mg)+Compound 1 (10 mg). Group (d) also had a high fat meal before THC administration. Endpoints were then evaluated as generally described in Example 1, and PK properties were measured as shown for the general methods of Example 2. It was found that a single low oral dose of Compound 1 (10 mg) administered 1 hour after a THC challenge rapidly reversed key psychotropic effects of THC doses as high as 30 mg, including a reduction in the visual analog scale (VAS) for feeling high (P=<0.0001) and improvement in VAS alertness (P=0.0042) and reduced body sway (P=0.0196). See Table 6.

In a pre-specified pooled analysis of data for the combined 21 mg or 30 mg THC dose levels, a single 10 mg dose of Compound 1 administered one hour after THC achieved statistical significance on all primary outcomes, including reduction in VAS feeling high (P=<0.0001), improvement in VAS alertness (P=0.0024), reduced body sway (P=0.0014), and reduction in heart rate (P=0.0125).

More particularly:

Reduction of Feeling High. Administration of oral THC alone produced a substantial increase in the VAS feeling high score. Either Coadministration of THC with Compound 1 or administration of Compound 1 one hour after the THC led to a highly significant reduction in feeling high compared to THC alone (overall p<0.0001). The 10 mg dose of Compound 1 was as effective as the 30 mg dose. The time course in both indicated that the drug reduced recovery time by several hours.

Effect on THC-Induced Body Sway. Administration of oral THC alone [either 21 mg or 30 mg] produced an increase in body sway. Coadministration of THC with Compound 1 or administration of Compound 1 after the administration of oral THC, showed a significant reduction in body sway. The 10 mg dose of Compound 1 was as effective as the 30 mg dose.

Improvement in Alertness. Administration of oral THC alone produced a substantial reduction in alertness. Coadministration of THC with Compound 1 or administration of Compound 1 after the administration of oral THC, showed a significant reduction in VAS alertness compared to administration of THC alone. The pooled analysis of both delayed administration cohorts and both simultaneous administration cohorts provided a similar result for both the 10 mg dose of Compound 1 and the 30 mg dose.

Produced Sustained Reduction of VAS Drug Effects. Administration of oral THC alone produced a substantial reduction in the 1] VAS feeling Good from the drug effect, 2] VAS feeling Bad from the drug effect, and 3] VAS feeling a drug effect. Administration of Compound 1 one hour after administration of THC led to a highly significant reduction in feeling any drug effect compared to THC alone). The pooled analysis of both delayed administration cohorts provided a similar result for both the 21 mg and 30 mg THC levels.

Responder Rate for Reduction in Feeling High. Subjects given THC [21 or 30 mg] and treated with 10 mg or 30 mg Compound 1 [administered either simultaneous with the THC or one hour after the THC] were responders. Coadministration of THC with Compound 1 led to a highly significant increase in the proportion of responders compared to THC alone. The 10 mg dose of Compound 1 was as effective as the 30 mg dose.

TABLE 6

| | THC Amount | | | | | |
|---|---|---|---|---|---|---|
| | 21 mg THC | 21 mg THC | 21 mg THC | 30 mg THC | 21 mg THC | 21-30 mg THC |
| | ANEB-001 Amount | | | | | |
| | 30 mg | 10 mg | 10 mg | 10 mg | 10-30 mg | 10 mg |
| | ANEB-001 | ANEB-001 | ANEB-001 | ANEB-001 | ANEB-001 | ANEB-001 |
| | THC/ANEB-001 dosage timing | | | | | |
| | Simultaneous | Simultaneous | 1 hr Delay | 1 hr Delay | Simultaneous | 1 hr Delay |
| PRIMARY OUTCOMES | | | | | | |
| VAS Feeling High | −0.7856 | −0.7072 | −0.5545 | −0.8581 | −0.7364 | −0.7028 |
| log(mm) | p = <0.0001 | p = 0.0001 | p = 0.0380 | p = <0.0001 | p = <0.0001 | p = <0.0001 |
| Body Sway (mm) | −30.6% | −29.3% | −3% | −34.8% | −30.0% | −32.4% |
| | p = 0.0023 | p = 0.0069 | p = 0.0406 | p = 0.0196 | p = 0.0013 | p = 0.0014 |
| VAS Alertness (mm) | 10.8 | 9.2 | 4.4 | 10.8 | 15.6 | 11.3 |
| | p = 0.0013 | p = 0.0047 | p = 0.4834 | p = 0.0042 | p = 0.0007 | p = 0.0024 |
| Heart Rate (supine) | −2.2 | −1.1 | 10.3 | 6.7 | 0.6 | −8.1 |
| (bpm) | p = 0.4730 | p = 0.7264 | p = 0.0589 | p = 0.1119 | p = 0.8247 | p = 0.0125 |
| SECONDARY/ | | | | | | |
| EXPLORATORY | | | | | | |
| OUTCOMES | | | | | | |
| VAS External | Effect | Effect | Effect | Effect | Effect | Effect |
| Perception log(mm) | Reversed | Reversed | Modified | Reversed | Reversed | Reversed |
| | p = 0.0010 | p = 0.0037 | p = 0.0530 | p = 0.0007 | p = 0.0004 | p = <0.0001 |
| VAS Internal | Effect | Effect | p = 0.2486 | Effect | Effect | Effect |
| Perception log(mm) | Reversed | Reversed | | Reversed | Reversed | Reversed |
| | p = 0.0016 | p = 0.0120 | | p = 0.0007 | p = 0.0012 | p = 0.0040 |
| VAS Feel drug effect | — | — | p = 0.2010 | Effect | — | Effect |
| | | | | Reversed | | Reversed |
| | | | | p = <0.0001 | | p = 0.0003 |
| VAS good drug effect | — | — | p = 0.0795 | Effect | — | Effect |
| | | | | Reversed | | Reversed |
| | | | | p = 0.0112 | | p = 0.0021 |
| VAS bad drug effect | — | — | p = 0.8695 | Effect | — | Effect |
| | | | | Reversed | | Reversed |
| | | | | p = 0.0003 | | p = 0.0274 |
| Responder Rate (VAS | Effect | Effect | Effect | Effect | Effect | Effect |
| Feeling High) | Reversed | Reversed | Reversed | Reversed | Reversed | Reversed |
| | p = 0.0019 | p = 0.0002 | p = <0.0001 | p = <0.0001 | p = 0.0005 | p = 0.0002 |

### Example 10—Synthesis of Compound 2

A metabolite of Compound 1 (Compound 2) was synthesized using the general methods described in U.S. Pat. No. 7,504,522 according to Scheme 1:

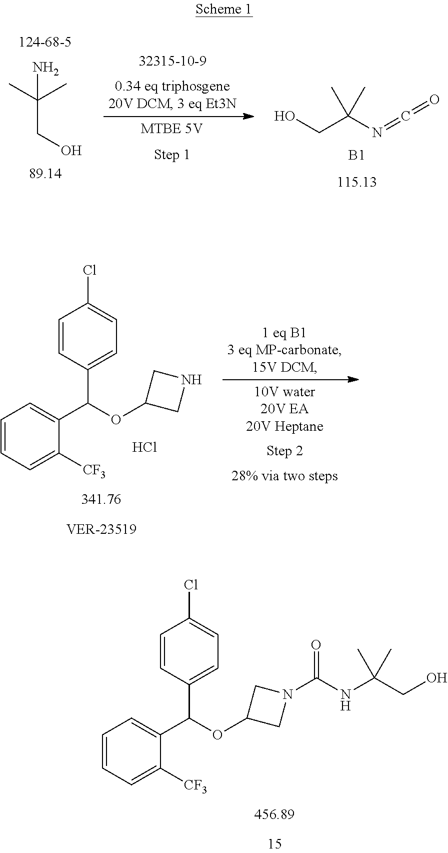

DCM=dichloromethane; Et3N=triethylamine; EA=ethyl acetate

Compound 2, 3-((4-chlorophenyl)(2-(trifluoromethyl)phenyl)methoxy)-N-(1-hydroxy-2-methylpropan-2-yl)azetidine-1-carboxamide was obtained as a solid with 99.6% purity (HPLC). 1HNMR (300 MHz, CDCl3): δ 7.70 (d, J=9, 1H), 7.57-7.64 (m, 2H), 7.45 (t, J=6, 1H), 7.30 (t, J=6, 2H), 7.28 (t, J=6, 2H), 5.78 (s, 1H), 4.33 (bs, 1H), 4.06 (t, J=6, 1H), 4.06 (bs, 1H), 3.93-3.97 (m, 3H), 3.80 (bs, 1H), 3.57 (s, 2H), 1.24 (s, 6H). HPLC retention time (210 nm): 14.220 min. LCMS (ES-API): 457.10 (M+H).

### Example 11—Synthesis of Compound 3

A metabolite of Compound 1 (Compound 3) was synthesized using the general methods described in U.S. Pat. No. 7,504,522 according to Scheme 2:

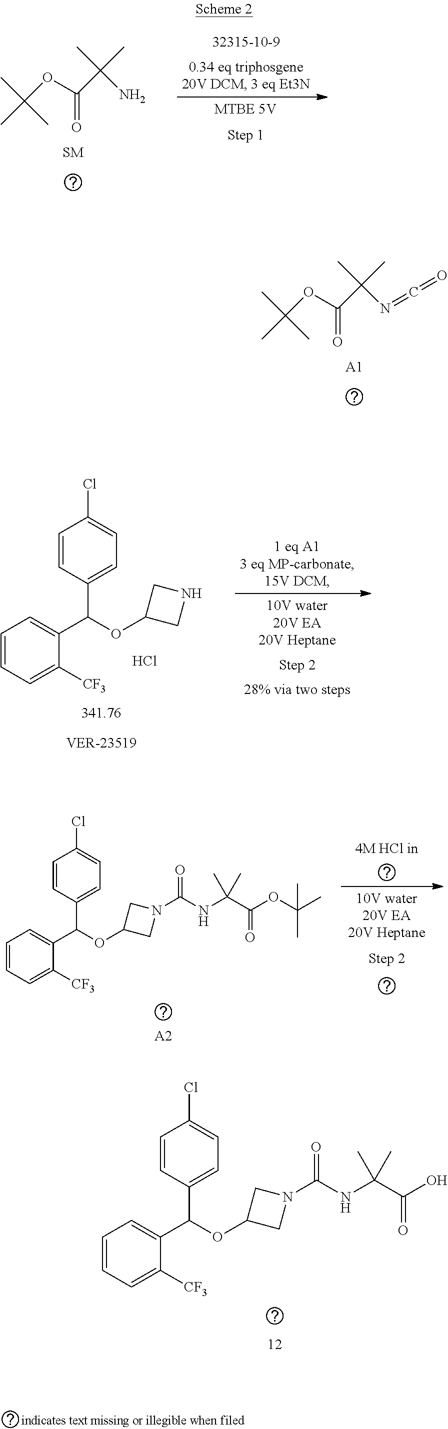

Compound 3, 2-(3-((4-chlorophenyl)(2-(trifluoromethyl)phenyl)methoxy)azetidine-1-carboxamido)-2-methylpropanoic acid, was obtained as a solid with 97.68% purity (HPLC). HNMR (300 MHz, CDCl3): δ 7.66 (d, J=6, 1H), 7.54-7.59 (m, 2H), 7.41 (t, J=6, 1H), 7.21-7.28 (m, 4H), 5.73 (s, 1H), 5.05 (bs, 1H), 4.24 (s, 1H), 4.03 (bs, 1H), 3.89-3.92 (m, 2H), 3.75 (bs, 1H), 1.36 (s, 6H). HPLC retention time (210 nm): 13.738 min. LCMS (ES-API): 471.10 (M+H).

### Example 12—Agonist and Antagonist Evaluation of Compounds

Compounds 2 and 3 were evaluated for activity against CB1 [CNR1] and CB2 [CNR2], both as agonists or as antagonists using the PathHunter GPCR Biosensor (Eurofins Discovery X, Fremont CA). For agonist assays, data was normalized to the maximal and minimal response observed in the presence of control ligand and vehicle. For antagonist assays, data was normalized to the maximal and minimal response observed in the presence of EC80 ligand and vehicle. The following EC80 concentrations were used: CNR1 cAMP: 0.0012 μM CP55940; CNR2 cAMP: 0.0048 μM CP55940. For Gi cAMP assays, the following forskolin concentration was used: CNR1 cAMP: 20 μM Forskolin; CNR2 cAMP: 25 μM Forskolin. The results are provided in Table 7. Both compounds are selective CB1 antagonists.

TABLE 7

| Compound | Description | Result [μM] |
|---|---|---|
| 2 | CNR1 Agonist EC50 | >1 |
| 2 | CNR1 Antagonist IC50 | 0.04562469 |
| 2 | CNR2 Agonist EC50 | >1 |
| 2 | CNR2 Antagonist IC50 | >1 |
| 3 | CNR1 Agonist EC50 | >1 |
| 3 | CNR1 Antagonist IC50 | 0.3827481 |
| 3 | CNR2 Agonist EC50 | >1 |
| 3 | CNR2 Antagonist IC50 | >1 |

### Example 13—Treatment with Compounds 2 or 3

Following the general methods of Example 9, Compound 1 is replaced with Compound 2 or 3 for treatment of cannabinoid overdose.

While some embodiments have been shown and described, various modifications and substitutions may be made thereto without departing from the spirit and scope of the present disclosure. For example, for claim construction purposes, it is not intended that the claims set forth hereinafter be construed in any way narrower than the literal language thereof, and it is thus not intended that exemplary embodiments from the specification be read into the claims. Accordingly, it is to be understood that the present disclosure has been described by way of illustration and not limitations on the scope of the claims.

What is claimed is:

1. A method of treating a known or suspected acute cannabinoid intoxication in a subject in need thereof, the method comprising: administering to the subject a therapeutically-effective amount of a compound having the structure 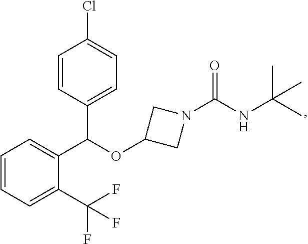 [SMILES: CC(C)(C)NC(=O)N1CC(OC(c2ccc(Cl)cc2)c2ccccc2C(F)(F)F)C1]
wherein treating improves one or more metrics associated with acute cannabinoid intoxication.

2. The method of claim 1, wherein the improvement is measured between a first time before administration of the compound and a second time after administration of the compound.

3. The method of claim 2, wherein the difference between the first time and the second time is 30 minutes to 8 hours.

4. The method of any one of claims 1-3, wherein the time between pre-administration and post administration of the compound is 30 minutes to 4 hours.

5. The method of any one of claims 1-4, wherein the metric comprises one or more of impaired motor coordination, and euphoria.

6. The method of any one of claims 1-5, wherein the metric comprises heartrate, loss of alertness, body movement, or visual analog scale (VAS) assessment of cannabinoid intoxication.

7. The method of claim 6, wherein the loss of alertness caused by cannabinoid intoxication is reduced by at least about 20%.

8. The method of claim 6, wherein loss of alertness is measured by the subject's responses to one or more questions asked by a healthcare provider or emergency responder.

9. The method of claim 8, wherein the one or more questions are directed to one or more of mood and feelings.

10. The method of claim 9, wherein the one or more questions are directed to one or more of alertness/fuzzy/clearheaded, coordinate/clumsy, lethargic/energetic, contented/discontented, troubled/tranquil, mentally slow/quick witted, tense/relaxed, attentive/dreamy, drowsy, calm/excited, strong/feeble, incompetent/proficient, happy/sad, antagonistic/friendly, interested/bored, withdrawn/sociable, self-centered/outward going or depressed/elated.

11. The method of claim 6, wherein the loss of alertness caused by cannabinoid intoxication is reduced by at least one level.

12. The method of claim 6, wherein the loss of alertness caused by cannabinoid intoxication is reduced by at least 10%.

13. The method of claim 6, wherein level of alertness is scored from using best verbal response.

14. The method of claim 6, wherein body movement comprises body sway.

15. The method of claim 6, wherein body sway is measured as a distance.

16. The method of claim 6, wherein body sway comprises antero-posterior sway (mm).

17. The method of claim 6, wherein body sway distance is measured using a Wright ataxiameter.

18. The method of claim 6, wherein the distance of body sway distance caused by cannabinoid intoxication is reduced by at least about 25%.

19. The method of claim 6, wherein VAS comprises measurement of one or more of alertness, negative affect, calmness, feeling high, and external perception.

20. The method of claim 6, wherein VAS comprises measurement of one or more of the sixteen items in the Bond & Lader scales.

21. The method of claim 1, wherein the metric is measured by a healthcare provider or emergency responder.

22. The method of claim 1, wherein the metric is self-reported by the subject.

23. A method of treating a known or suspected acute cannabinoid intoxication in a subject in need thereof, the method comprising: administering to the subject a therapeutically-effective amount of a compound having the structure 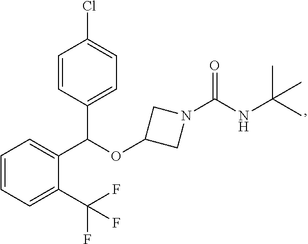 [SMILES: CC(C)(C)NC(=O)N1CC(OC(c2ccc(Cl)cc2)c2ccccc2C(F)(F)F)C1]
wherein the compound has a plasma Cₘₐₓ of about 100-500 ng/mL.

24. A method of treating a known or suspected acute cannabinoid intoxication in a subject in need thereof, the method comprising: administering to the subject a therapeutically-effective amount of a compound having the structure 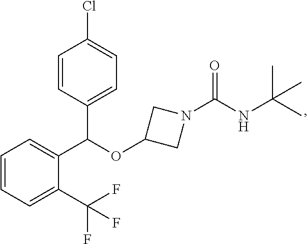 [SMILES: CC(C)(C)NC(=O)N1CC(OC(c2ccc(Cl)cc2)c2ccccc2C(F)(F)F)C1]
wherein the compound has a plasma AUCₗₐₛₜ of about 500-2800 h*ng/mL.

25. A method of treating a known or suspected acute cannabinoid intoxication in a subject in need thereof, the method comprising: administering to the subject a therapeutically-effective amount of a compound having the structure 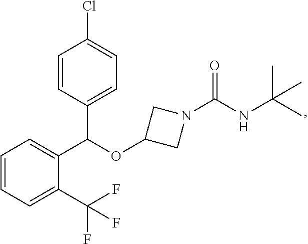 [SMILES: CC(C)(C)NC(=O)N1CC(OC(c2ccc(Cl)cc2)c2ccccc2C(F)(F)F)C1]
wherein the compound has a plasma AUC5h of about 100-1200 h*ng/mL.

26. A method of treating a known or suspected acute cannabinoid intoxication in a subject in need thereof, the method comprising: administering to the subject a therapeutically-effective amount of a compound having the structure 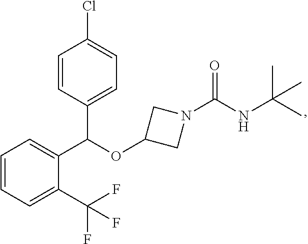 [SMILES: CC(C)(C)NC(=O)N1CC(OC(c2ccc(Cl)cc2)c2ccccc2C(F)(F)F)C1]
wherein the compound has a dose normalized plasma Cₘₐₓ of about 3-20 ng/mL/mg.

27. A method of treating a known or suspected acute cannabinoid intoxication in a subject in need thereof, the method comprising: administering to the subject a therapeutically-effective amount of a compound having the structure 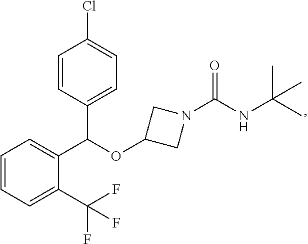 [SMILES: CC(C)(C)NC(=O)N1CC(OC(c2ccc(Cl)cc2)c2ccccc2C(F)(F)F)C1]
wherein the compound has a dose normalized plasma AUCₗₐₛₜ of about 50-400 h*ng/mL/mg.

28. The method of claim 27 wherein the compound has a dose normalized plasma AUCₗₐₛₜ of about 50-200 h*ng/mL/mg.

29. A method of treating a known or suspected acute cannabinoid intoxication in a subject in need thereof, the method comprising: administering to the subject a therapeutically-effective amount of a compound having the structure 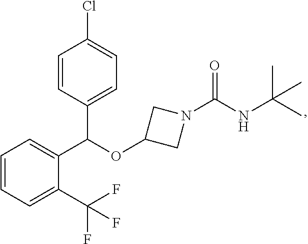 [SMILES: CC(C)(C)NC(=O)N1CC(OC(c2ccc(Cl)cc2)c2ccccc2C(F)(F)F)C1]
wherein the compound has a dosage normalized plasma AUC5h of about 10-40 h*ng/mL/mg.

30. A method of treating a known or suspected acute cannabinoid intoxication in a subject in need thereof, the method comprising: administering to the subject a therapeutically-effective amount of a compound having the structure 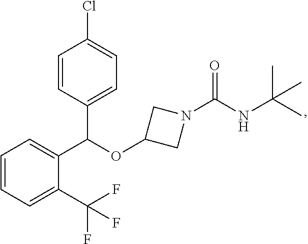 [SMILES: CC(C)(C)NC(=O)N1CC(OC(c2ccc(Cl)cc2)c2ccccc2C(F)(F)F)C1]
wherein the compound has a Tmax of or less than about 60 minutes.

31. The method of any one of claims 1-30, wherein the amount of compound administered to the subject is 1-200 mg, 10-50 mg, 10-30 mg, or 20-30 mg.

32. The method of any one of claims 1-30, wherein the method further comprises administering 5-300 mg, 5-100 mg, or 10-30 mg of delta-9-tetrahydrocannabinol (THC) to the subject.

33. The method of any one of claims 1-30, wherein the THC and the compound are administered at the same time.

34. The method of any one of claims 1-30, wherein the THC is administered 10 min to 8 hours before the compound is administered.

35. The method of any one of claims 1-34, wherein the administration is oral.

36. The method of any one of claims 1-35, wherein the compound is administered as a capsule.

37. The method of claim 36, wherein the capsule comprises a soft gel capsule.

38. The method of any one of claims 1-37 wherein the acute cannabinoid intoxication results from ingestion of an edible form of Cannabis.

39. A method of treating a known or suspected acute cannabinoid intoxication in a subject in need thereof, the method comprising: administering to the subject a therapeutically-effective amount of a compound having the structure 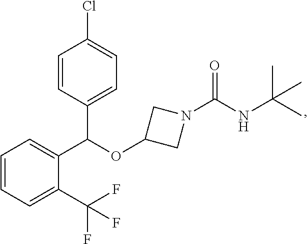 [SMILES: CC(C)(C)NC(=O)N1CC(OC(c2ccc(Cl)cc2)c2ccccc2C(F)(F)F)C1]
wherein treating accelerates recovery, shortens time in medical care or reduces the effects the intoxication as determined by an acceptable metric evaluation.

40. A composition comprising a compound having the structure:

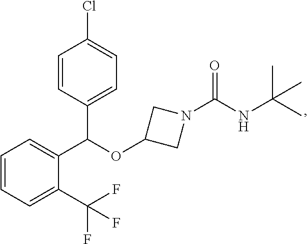 [SMILES: CC(C)(C)NC(=O)N1CC(OC(c2ccc(Cl)cc2)c2ccccc2C(F)(F)F)C1]

41. The composition of claim 40, wherein the compound is a salt, polymorph, solvate, hydrate, stereoisomer, or isotope thereof.

42. The composition of claim 40, wherein the compound is a solid.

43. The composition of claim 40, wherein the compound is a crystalline solid.

44. A pharmaceutical composition comprising the compound of claim 40 and a pharmaceutically acceptable excipient.

45. An oral dosage form of the compound of claim 40 and a pharmaceutically acceptable excipient.

46. An injectable dosage form of the compound of claim 40 and a pharmaceutically acceptable excipient.

47. A method of treating a known or suspected acute cannabinoid intoxication in a subject in need thereof, the method comprising: administering to the subject a therapeutically-effective amount of a compound of any one of claims 40-46.
